(19) United States
(12) Patent Application Publication
(10) Pub. No.: US 2024/0165166 A1
(43) Pub. Date: May 23, 2024

(54) PRIMED PLACENTAL TISSUE AND USES IN REGENERATIVE MEDICINE

(71) Applicant: OSIRIS THERAPEUTICS, INC., Columbia, MD (US)

(72) Inventors: Chaoyang LI, Columbia, MD (US); Vimal JACOB, Columbia, MD (US); Min Sung PARK, Columbia, MD (US); Sandeep DHALL, Columbia, MD (US); Malathi SATHYAMOORTHY, Columbia, MD (US)

(21) Appl. No.: 18/043,064

(22) PCT Filed: Sep. 30, 2021
(86) PCT No.: PCT/IB2021/059024

§ 371 Date: Feb. 27, 2023

Related U.S. Application Data
(60) Provisional application No. 63/088,065, filed on Oct. 6, 2020.

(51) Int. Cl. A61K 35/50; A61K 35/51; A61P 9/10; A61P 17/02

(52) U.S. Cl. CPC A61K 35/50; A61K 35/51; A61P 9/10; A61P 17/02

(57) ABSTRACT

Disclosed are methods and compositions useful for regenerative medicine for the therapeutic regeneration and repair of cells, tissues, or organs. The present disclosure relates to methods of priming viable placental tissue, primed viable placental tissue, and products thereof. Various priming techniques are disclosed including exposure viable placental tissue to hypoxia, UV light, bioactive materials, or combinations thereof.

### CROSS-REFERENCE WITH RELATED APPLICATIONS

This application claims the benefit of U.S. Provisional Application No. 63/088,065 filed Oct. 6, 2020, the contents of which are incorporated into the present application by reference.

### FIELD OF THE INVENTION

The present invention generally relates to the field of regenerative medicine for the therapeutic regeneration and repair of cells, tissues, or organs.

### BACKGROUND OF THE INVENTION

Regenerative medicine generally involves the process of regenerating, replacing, or repairing damaged or defective cells, tissues, and/or organs in the body to restore their normal function. Examples of regenerative medicine include cell-based therapies, biologically active molecule-based therapies, and tissue engineering.

Mesenchymal stromal cells (MSCs) have exhibited therapeutic regenerative functions, including angiogenic, anti-inflammatory, anti-oxidative, antimicrobial, and anti-fibrotic properties, when used in cell-based therapies. Methods to enhance the regenerative functions of the MSCs prior to use have been studied using in vitro models of cell priming. These methods involve isolating the MSCs from tissue either by enzymatic digestion or other methods and can further involve the passaging of the isolated cells to expand the cell number. The isolated/expanded cells are then exposed to hypoxic conditions, UV light, culture conditions, incubation, or other cell priming conditions. However, the process of isolating and expanding the MSCs can reduce their stemness and other therapeutically beneficial properties. Moreover, other therapeutically beneficial native cells, such as fibroblasts and epithelial cells, that may be present within the tissue are lost during the isolation of the MSCs. Thus, cell-based therapies containing primed MSCs have limited therapeutic effects.

The placenta contains extracellular matrix (ECM), viable native cells (including MSCs, fibroblasts, and epithelial cells), cytokines, growth factors, and other nutrients which makes it a potent solution for a variety of indications in regenerative medicine. Viable placental tissue can include tissue from the amnion, chorion, and/or umbilical cord and includes viable cells native to the tissue. Viable placental tissue and viable placental tissue products are currently used in the management of indications in wound treatment, orthopedics, sports medicine, ear/nose/throat (ENT), trauma, dental, and other fields where regenerative medicine is useful due to the angiogenic, anti-inflammatory, anti-oxidative, antimicrobial, and anti-fibrotic properties of viable placental tissues. However, methods to enhance or boost the therapeutic regenerative properties of viable placental tissue have not been previously explored.

### SUMMARY OF THE INVENTION

The present disclosure provides a solution to the aforementioned limitations and deficiencies in the art generally relating to regenerative medicine and particularly related to regenerative medicine using viable placental tissue or placental tissue products. The solution is premised on the priming of viable placental tissue by exposing the viable placental tissue to priming conditions such as hypoxia, UV light, bioactive materials, or culture conditions, or combinations thereof.

The priming of viable placental tissue results in the enhancement or boost of the therapeutic regenerative properties of the viable placental tissue when used in regenerative medicine. These enhanced therapeutic regenerative properties include increased cellular activity, as well as the increased production of growth factors, peptides, antimicrobial peptides, cytokines, extracellular vesicles, exosomes, secretomes, microvesicles, extracellular matrix (ECM), and other bioactive materials, all of which play key roles in regenerative medicine. During the priming process, the native viable placental cells, including mesenchymal stromal cells (MSCs), fibroblasts, and epithelial cells, have the benefit of being surrounded and embedded in their native placental tissue which is rich in a mixture of nutrients, proteins and signaling molecules. Because the MSCs are not isolated from their native placental tissue, the MSCs will maintain/preserve their stemness and other beneficial therapeutic properties during the priming process. In addition, the other beneficial viable native cells, e.g. fibroblasts and epithelial cells, will still be present in the placental tissue which includes the native ECM. It is known that ECM is not just a structural scaffold but is actively involved in providing signaling cues to cells and plays a dynamic role in cellular behavior to repair damaged tissue. Hence the primed viable placental tissue including its native ECM, viable native cells, growth factors and other bioactive materials, as a whole, can be utilized for production and can play a critical role in regenerative medicine. For example, primed viable placental tissue and products made from primed viable placental tissue can be used for their boosted therapeutic regenerative properties in sports medicine, orthopedics, trauma, ENT, dental, tissue regeneration, wound treatments (including chronic wounds), or any other indications that require therapeutic regenerative properties of a product.

In one aspect of the disclosure, disclosed is a composition comprising hypoxia primed viable placental tissue. In some embodiments, the hypoxia primed viable placental tissue exhibits enhanced angiogenic factor (VEGF-A) function (preferably about a 3 fold to about a 5 fold increase in activity) as determined by a cellular functional assay, an increase in VEGF-A secretion (preferably about a 1 fold to about a 3 fold increase), and/or an increase in IL-1RA secretion (preferably about a 1 fold to about a 3 fold increase) as compared to non-primed viable placental tissue cultured under normoxia conditions. In some embodiments, the hypoxia primed viable placental tissue is amnion tissue, chorion tissue, or umbilical cord tissue, or mixtures thereof. In some embodiments, the hypoxia primed viable placental tissue comprises one or more of mesenchymal stromal cells (MSCs), epithelial cells, or fibroblasts. In some embodiments, the hypoxia primed viable placental tissue is in the form of a sheet, wrap, or graft. In other embodiments, the hypoxia primed viable placental tissue is in the form of minced pieces or a powder. In some embodiments, the composition further comprises a pharmaceutically acceptable carrier. In some embodiments, the pharmaceutically acceptable carrier is a suspension, solution, gel, paste, emulsion, cream, or powder. In some embodiments, the pharmaceutically acceptable carrier comprises one or more of a saline solution, a buffer solution, a sugar, trehalose, a protein, or a starch. In some embodiments, the composition further comprises one or more bioactive materials. In some embodiments, the one or more bioactive materials comprise extracellular vesicles, exosomes, microvesicles, secretomes, cytokines, growth factors, peptides, antimicrobial peptides, extracellular matrices, TNF-alpha (preferably at an amount from about 5 to about 50 ng/ml), interferon-gamma (preferably at an amount from about 5 to about 50 ng/ml), or nanoparticles thereof. In some embodiments, the one or more bioactive materials are the isolated bioactive materials produced by the priming methods using hypoxic conditions disclosed herein. In some embodiments, the composition is cryopreserved or lyophilized.

In another aspect of the disclosure, disclosed is a method for priming viable placental tissue, the method comprising: (a) providing the viable placental tissue, wherein the viable placental tissue comprises viable cells native to the viable placental tissue, (b) contacting the viable placental tissue with a culture medium at hypoxic conditions, thereby priming the viable placental tissue and generating spent culture medium, and (c) optionally, separating the primed viable placental tissue from the spent culture medium. In some embodiments, the hypoxic conditions are from about 1% to about 5% 02 or from about 1% to about 3% 02, or about 2% 02.

In some embodiments, step (b) is conducted for from about 4 to about 96 hours; and/or is conducted in a hypoxia chamber or a hypoxia incubator. In some embodiments, the viable placental tissue is amnion tissue, chorion tissue, umbilical cord tissue, or mixtures thereof. In some embodiments, the viable cells comprise one or more of mesenchymal stromal cells (MSCs), epithelial cells, or fibroblasts. In some embodiments, the viable placental tissue is in the form of a sheet, wrap, or graft. In other embodiments, the viable placental tissue is in the form of minced pieces or a powder. In some embodiments, the culture medium comprises a chemically defined culture medium; and/or comprises nutrients; and/or comprises lysed platelets; and/or comprises serum. In some embodiments, the serum is fetal bovine serum (FBS) or human serum albumin. In some embodiments, the culture medium comprises lysed platelets. In some embodiments, the culture medium comprises DMEM. In some embodiments, the method further comprises adding an effective amount of one or more bioactive materials to the culture medium in step 1(b). In some embodiments, the one or more added bioactive materials comprise growth factors, peptides, antimicrobial peptides, cytokines, enzymes, extracellular vesicles, extracellular matrices, TNF-alpha (preferably at an amount from about 5 to about 50 ng/ml), interferon-gamma (preferably at an amount from about 5 to about 50 ng/ml), or nanoparticles thereof. In some embodiments, the hypoxia primed viable placental tissue exhibits enhanced angiogenic factor (VEGF-A) function (preferably about a 3 fold to about a 5 fold increase in activity) as determined by a cellular functional assay, an increase in VEGF-A secretion (preferably about a 1 fold to about a 3 fold increase), and/or an increase in IL-1RA secretion (preferably about a 1 fold to about a 3 fold increase) as compared to non-primed viable placental tissue cultured under normoxia conditions. In some embodiments, step (c) is not conducted and the hypoxia primed viable placental tissue remains with the spent culture medium. In other embodiments, step (c) is conducted and the hypoxia primed viable placental tissue is separated from the spent culture medium. In some embodiments, the method further comprises collecting the spent culture medium of step (c). In some embodiments, the method further comprises isolating one or more bioactive materials from the spent culture medium. In some embodiments, the isolated one or more bioactive materials comprise extracellular vesicles, exosomes, microvesicles, secretomes, cytokines, growth factors, peptides, antimicrobial peptides, or extracellular matrices.

In another aspect of the disclosure, disclosed is a composition comprising the hypoxia primed viable placental tissue produced by the priming methods using hypoxic conditions disclosed herein. In some embodiments the hypoxia primed viable placental tissue is in the form of a sheet, wrap, or graft. In other embodiments, the hypoxia primed viable placental tissue is in the form of minced pieces or a powder. In other embodiments, the composition further comprises a pharmaceutically acceptable carrier which can be a suspension, solution, gel, paste, emulsion, cream, or powder. In some embodiments, the pharmaceutically acceptable carrier comprises one or more of a saline solution, a buffer solution, a sugar, trehalose, a protein, or a starch. In other embodiments, the composition is cryopreserved or lyophilized.

In still another aspect of the disclosure, disclosed is a composition comprising the spent culture media and/or the one or more isolated bioactive materials produced by the priming methods using hypoxic conditions disclosed herein. In some embodiments, the composition further comprises a pharmaceutically acceptable carrier which can be a suspension, solution, gel, paste, emulsion, cream, or powder. In some embodiments, the pharmaceutically acceptable carrier comprises one or more of a saline solution, a buffer solution, a sugar, trehalose, a protein, or a starch. In other embodiments, the composition is cryopreserved or lyophilized.

In another aspect of the disclosure, disclosed is a method for priming viable placental tissue, the method comprising: (a) providing the viable placental tissue, wherein the viable placental tissue comprises viable cells native to the viable placental tissue, and (b) exposing the viable placental tissue to UV light, thereby priming the viable placental tissue. In some embodiments, the UV light is UV-A light, or UV-B light, or combinations thereof. In some embodiments, the UV light is UVB light. In some embodiments, the exposure time is from about 10 seconds to about 4 minutes, or from about 20 seconds to about 180 seconds. In some embodiments, the viable placental tissue is amnion tissue, chorion tissue, or umbilical cord tissue, or mixtures thereof. In some embodiments, the viable cells comprise one or more of mesenchymal stromal cells (MSCs), epithelial cells, or fibroblasts. In some embodiments, the viable placental tissue is in the form of a sheet, wrap, or graft. In other embodiments, the viable placental tissue is in the form of minced pieces or a powder. In some embodiments, the method further comprises (c) contacting the primed viable placental tissue with a culture medium and thereby generating spent culture medium, and (d) optionally, separating the UV light primed viable placental tissue from the spent culture medium. In some embodiments, step (c) is conducted for from about 4 to about 96 hours, or from about 24 to about 96 hours; and/or is conducted in an incubator. In some embodiments, the culture medium comprises a chemically defined culture medium; and/or comprises nutrients; and/or comprises lysed platelets; and/or comprises serum. In some embodiments, the serum is fetal bovine serum (FBS) or human serum albumin. In some embodiments, culture medium comprises lysed platelets. In some embodiments, the culture medium comprises DMEM. In some embodiments, the method further comprises adding an effective amount of one or more bioactive materials to the culture medium in step (c). In some embodiments, the added one or more bioactive materials comprise growth factors, peptides, antimicrobial peptides, cytokines, enzymes, extracellular vesicles, extracellular matrices, TNF-alpha (preferably at an amount from about 5 to about 50 ng/ml), interferon-gamma (preferably at an amount from about 5 to about 50 ng/ml), or nanoparticles thereof. In some embodiments, the UV light primed viable placental tissue exhibits an increased HBD-2 secretion (preferably about a 1 fold to about a 4 fold increase) as compared to viable placental tissue not exposed to UV light (non-primed). In some embodiments, step (d) is not conducted and the UV light primed viable placental tissue remains with the spent culture medium. In other embodiments, step (d) is conducted and the UV light primed viable placental tissue is separated from the spent culture medium. In some embodiments, the method further comprises collecting the spent culture medium of step (d).

In some embodiments, the method further comprises isolating one or more bioactive materials from the spent culture medium. In some embodiments, the isolated one or more bioactive materials comprise extracellular vesicles, exosomes, microvesicles, secretomes, cytokines, growth factors, peptides, antimicrobial peptides, or extracellular matrices.

In another aspect of the disclosure, disclosed is a composition comprising the UV light primed viable placental tissue produced by the priming methods using UV light disclosed herein. In some embodiments the UV light primed viable placental tissue is in the form of a sheet, wrap, or graft. In other embodiments, the UV light primed viable placental tissue is in the form of minced pieces or a powder. In other embodiments, the composition further comprises a pharmaceutically acceptable carrier which can be a suspension, solution, gel, paste, emulsion, cream, or powder. In some embodiments, the pharmaceutically acceptable carrier comprises one or more of a saline solution, a buffer solution, a sugar, trehalose, a protein, or a starch. In other embodiments, the composition is cryopreserved or lyophilized.

In another aspect of the disclosure, disclosed is a composition comprising UV light primed viable placental tissue. In some embodiments, the UV light primed placental tissue exhibits an increased HBD-2 secretion (preferably about a 1 fold to about a 4 fold increase) as compared to viable placental tissue not exposed to UV light (non-primed). In some embodiments, the UV light primed viable placental tissue is amnion tissue, chorion tissue, or umbilical cord tissue, or mixtures thereof. In some embodiments, the UV light primed viable placental tissue comprises one or more of MSCs, epithelial cells, or fibroblasts. In some embodiments, the UV light primed viable placental tissue is in the form of a sheet, wrap, or graft. In other embodiments, the UV light primed viable placental tissue is in the form of minced pieces or a powder. In some embodiments, the composition further comprises a pharmaceutically acceptable carrier. In some embodiments, the pharmaceutically acceptable carrier is a suspension, solution, gel, paste, emulsion, cream, or powder. In some embodiments, the pharmaceutically acceptable carrier comprises one or more of a saline solution, a buffer solution, a sugar, trehalose, a protein, or a starch. In some embodiments, the composition further comprises one or more bioactive materials. In some embodiments, the one or more bioactive materials comprise extracellular vesicles, exosomes, microvesicles, secretomes, cytokines, growth factors, peptides, antimicrobial peptides, extracellular matrices, TNF-alpha (preferably at an amount from about 5 to about 50 ng/ml), interferon-gamma (preferably at an amount from about 5 to about 50 ng/ml), or nanoparticles thereof. In some embodiments, the one or more bioactive materials are the isolated bioactive materials produced by the priming methods using UV light disclosed herein. In some embodiments, the composition is cryopreserved or lyophilized.

In still another aspect of the disclosure, disclosed is a composition comprising the spent culture medium and/or the one or more isolated bioactive materials produced by the priming methods using UV light disclosed herein. In some embodiments, the composition further comprises a pharmaceutically acceptable carrier which can be a suspension, solution, gel, paste, emulsion, cream, or powder. In some embodiments, the pharmaceutically acceptable carrier comprises one or more of a saline solution, a buffer solution, a sugar, trehalose, a protein, or a starch. In other embodiments, the composition is cryopreserved or lyophilized.

In another aspect of the disclosure, disclosed is a method for priming viable placental tissue, the method comprising: (a) providing the viable placental tissue, wherein the viable placental tissue comprises viable cells native to the viable placental tissue, (b) contacting the tissue with a culture medium comprising an effective amount of one or more bioactive materials, thereby priming the viable placental tissue and generating spent culture medium, and (c) optionally, separating the bioactive material primed viable placental tissue from the spent culture medium. In some embodiments, the one or more bioactive materials comprise growth factors, peptides, antimicrobial peptides, cytokines, enzymes, extracellular vesicles, extracellular matrices, TNF-alpha, interferon-gamma, or nanoparticles thereof. In some embodiments, the one or more bioactive materials comprise TNF-alpha at an amount of about 5 to about 50 ng/ml, interferon-gamma at an amount of about 5 to about 50 ng/ml, or a combination of TNF-alpha at an amount of about 5 to about 50 ng/ml and interferon-gamma at an amount of about 5 to about 50 ng/ml. In some embodiments, step (b) is conducted from about 4 to about 96 hours; and/or in an incubator. In some embodiments, the viable placental tissue is amnion tissue, chorion tissue, umbilical cord tissue, or mixtures thereof. In some embodiments, the viable cells comprise one or more of mesenchymal stromal cells (MSCs), epithelial cells, or fibroblasts. In some embodiments, the viable placental tissue is in the form of a sheet, wrap, or graft. In other embodiments, the viable placental tissue is in the form of minced pieces or a powder. In some embodiments, the culture medium comprises a chemically defined culture medium; and/or comprises nutrients; and/or comprises lysed platelets; and/or comprises serum. In some embodiments, the serum is fetal bovine serum (FBS) or human serum albumin. In some embodiments, the culture medium comprises lysed platelets. In some embodiments, the culture medium comprises DMEM. In some embodiments, the bioactive material primed viable placental tissue exhibits one or more increased therapeutic regenerative properties comprising angiogenesis, anti-inflammatory, chemoattractant, antimicrobial, antioxidant or antifibrosis as compared to non-primed viable placental tissue as determined in vitro such as with ELISA and/or Multi-plex analysis, and/or in vivo. In some embodiments, step (c) is not conducted and the bioactive material primed viable placental tissue remains with the spent culture medium. In other embodiments, step (c) is conducted and the bioactive material primed viable placental tissue is separated from the spent culture medium. In some embodiments, the method further comprises collecting the spent culture medium of step (c) and isolating from the spent culture medium one or more bioactive materials present and/or formed in the culture medium in step (b). In some embodiments, the isolated one or more bioactive materials comprise extracellular vesicles, exosomes, microvesicles, secretomes, cytokines, growth factors, peptides, antimicrobial peptides, or extracellular matrices.

In another aspect of the disclosure, disclosed is a composition comprising the bioactive material primed viable placental tissue produced by the priming methods using bioactive materials disclosed herein. In some embodiments the bioactive material primed viable placental tissue is in the form of a sheet, wrap, or graft. In other embodiments, the bioactive material primed viable placental tissue is in the form of minced pieces or a powder. In other embodiments, the composition further comprises a pharmaceutically acceptable carrier which can be a suspension, solution, gel, paste, emulsion, cream, or powder. In some embodiments, the pharmaceutically acceptable carrier comprises one or more of a saline solution, a buffer solution, a sugar, trehalose, a protein, or a starch. In other embodiments, the composition is cryopreserved or lyophilized.

In another aspect of the disclosure, disclosed, is a composition comprising bioactive material primed viable placental tissue. In some embodiments, the bioactive material primed viable placental tissue exhibits one or more increased therapeutic regenerative properties comprising angiogenesis, anti-inflammatory, chemoattractant, antimicrobial, antioxidant or antifibrosis as compared to non-primed viable placental tissue as determined in vitro such as with ELISA and/or Multi-plex analysis, and/or in vivo. In some embodiments, the bioactive material primed viable placental tissue is amnion tissue, chorion tissue, or umbilical cord tissue, or mixtures thereof. In some embodiments, the bioactive material primed viable placental tissue comprises one or more of MSCs, epithelial cells, or fibroblasts. In some embodiments, the bioactive material primed viable placental tissue is in the form of a sheet, wrap, or graft. In other embodiments, the bioactive material primed viable placental tissue is in the form of minced pieces or a powder. In some embodiments, the composition further comprises a pharmaceutically acceptable carrier. In some embodiments, the pharmaceutically acceptable carrier is a suspension, solution, gel, paste, emulsion, cream, or powder. In some embodiments, the pharmaceutically acceptable carrier comprises one or more of a saline solution, a buffer solution, a sugar, trehalose, a protein, or a starch. In some embodiments, the composition further comprises one or more bioactive materials. In some embodiments, the one or more bioactive materials comprise extracellular vesicles, exosomes, microvesicles, secretomes, cytokines, growth factors, peptides, antimicrobial peptides, extracellular matrices, TNF-alpha (preferably at an amount from about 5 to about 50 ng/ml), interferon-gamma (preferably at an amount from about 5 to about 50 ng/ml), or nanoparticles thereof. In some embodiments, the one or more bioactive materials are the isolated bioactive materials produced by the priming methods using bioactive materials disclosed herein. In some embodiments, the composition is cryopreserved or lyophilized.

In still another aspect of the disclosure, disclosed is a composition comprising the spent culture medium and/or the one or more isolated bioactive materials produced by the priming methods using bioactive materials disclosed herein. In some embodiments, the composition further comprises a pharmaceutically acceptable carrier which can be a suspension, solution, gel, paste, emulsion, cream, or powder. In some embodiments, the pharmaceutically acceptable carrier comprises one or more of a saline solution, a buffer solution, a sugar, trehalose, a protein, or a starch. In other embodiments, the composition is cryopreserved or lyophilized.

In another aspect of the disclosure, disclosed is a method for priming viable placental tissue, the method comprising: (a) providing the viable placental tissue, wherein the viable placental tissue comprises viable cells native to the viable placental tissue, (b) exposing the viable placental tissue to UV light, (c) contacting the viable placental tissue with a culture medium at hypoxic conditions, thereby priming the viable placental tissue and generating spent culture medium, and (d) optionally, separating the UV light plus hypoxia primed viable placental tissue from the spent culture medium. In some embodiments, the UV light is UV-A light, or UV-B light, or combinations thereof. In some embodiments, the hypoxic conditions are from about 1% to about 5% 02, or from about 1% to about 3% 02, or about 2% 02. In some embodiments, step (b) is conducted prior to step (c). In other embodiments, step (c) is conducted prior to step (b). In some embodiments, step (b) and step (c) are conducted at the same time. In some embodiments, step (c) is conducted from about 4 to about 96 hours; and/or is conducted in a hypoxia chamber or a hypoxia incubator. In some embodiments, the viable placental tissue is amnion tissue, chorion tissue, umbilical cord tissue, or mixtures thereof. In some embodiments, the viable cells comprise one or more of mesenchymal stromal cells (MSCs), epithelial cells, or fibroblasts. In some embodiments, the viable placental tissue is in the form of a sheet, wrap, or graft. In other embodiments, the viable placental tissue is in the form of minced pieces or a powder. In some embodiments, the culture medium comprises a chemically defined culture medium; and/or comprises nutrients; and/or comprises lysed platelets; and/or comprises serum. In some embodiments, the serum is fetal bovine serum (FBS) or human serum albumin. In some embodiments, the culture medium comprises lysed platelets. In some embodiments, the culture medium comprises DMEM. In some embodiments, one or more bioactive materials are added to the culture medium in step (c). In some embodiments, the added one or more bioactive materials comprise growth factors, peptides, antimicrobial peptides, cytokines, enzymes, extracellular vesicles, extracellular matrices, TNF-alpha (preferably at an amount from about 5 to about 50 ng/ml), interferon-gamma (preferably at an amount from about 5 to about 50 ng/ml), or nanoparticles thereof. In some embodiments, the UV light plus hypoxia primed viable placental tissue exhibits one or more increased therapeutic regenerative properties comprising an increase in HBD-2 secretion (preferably about a 1 fold to about a 4 fold increase); enhanced angiogenic factor (VEGF-A) function (preferably about a 3 fold to about a 5 fold increase in activity) as determined by a cellular functional assay; an increase in VEGF-A secretion (preferably about a 1 fold to about a 3 fold increase); and/or an increase in IL-1RA secretion (preferably about a 1 fold to about a 3 fold increase) as compared to viable placental tissue not exposed to hypoxic conditions and UV light (non-primed). In some embodiments, step (d) is not conducted and the UV light plus hypoxia primed viable placental tissue remains with the spent culture medium. In other embodiments, the step (d) is conducted and the UV light plus hypoxia primed viable placental tissue is separated from the spent culture medium. In some embodiments, the method further comprises collecting the spent culture medium of step (d). In some embodiments, the method further comprises isolating one or more bioactive materials from the spent culture medium. In some embodiments, the isolated one or more bioactive materials comprise extracellular vesicles, exosomes, microvesicles, secretomes, cytokines, growth factors, peptides, antimicrobial peptides, or extracellular matrices.

In another aspect of the disclosure, disclosed is a composition comprising the UV light plus hypoxia primed viable placental tissue produced by the priming methods using UV light plus hypoxic conditions disclosed herein. In some embodiments the UV light plus hypoxia primed viable placental tissue is in the form of a sheet, wrap, or graft. In other embodiments, the UV light plus hypoxia primed viable placental tissue is in the form of minced pieces or a powder. In other embodiments, the composition further comprises a pharmaceutically acceptable carrier which can be a suspension, solution, gel, paste, emulsion, cream, or powder. In some embodiments, the pharmaceutically acceptable carrier comprises one or more of a saline solution, a buffer solution, a sugar, trehalose, a protein, or a starch. In other embodiments, the composition is cryopreserved or lyophilized.

In another aspect of the disclosure, disclosed is a composition comprising UV light plus hypoxia primed viable placental tissue. In some embodiments, the UV light plus hypoxia primed viable placental tissue exhibits one or more increased therapeutic regenerative properties comprising an increase in HBD-2 secretion (preferably about a 1 fold to about a 4 fold increase); enhanced angiogenic factor (VEGF-A) function (preferably about a 3 fold to about a 5 fold increase in activity) as determined by a cellular functional assay; an increase in VEGF-A secretion (preferably about a 1 fold to about a 3 fold increase); and/or an increase in IL-1RA secretion (preferably about a 1 fold to about a 3 fold increase) as compared to viable placental tissue not exposed to hypoxic conditions and UV light (non-primed). In some embodiments, the UV light plus hypoxia primed viable placental tissue is amnion tissue, chorion tissue, or umbilical cord tissue, or mixtures thereof. In some embodiments, the UV light plus hypoxia primed viable placental tissue is in the form of a sheet, wrap, or graft. In other embodiments, the UV light plus hypoxia primed viable placental tissue is in the form of minced pieces or a powder. In some embodiments, the composition further comprises a pharmaceutically acceptable carrier. In some embodiments, the pharmaceutically acceptable carrier is a suspension, solution, gel, paste, emulsion, cream, or powder. In some embodiments, the pharmaceutically acceptable carrier comprises one or more of a saline solution, a buffer solution, a sugar, trehalose, a protein, or a starch. In some embodiments, the composition further comprises one or more bioactive materials. In some embodiments, the one or more bioactive materials comprise extracellular vesicles, exosomes, microvesicles, secretomes, cytokines, growth factors, peptides, antimicrobial peptides, extracellular matrices, TNF-alpha (preferably at an amount from about 5 to about 50 ng/ml), interferon-gamma (preferably at an amount from about 5 to about 50 ng/ml), or nanoparticles thereof. In some embodiments, the one or more bioactive materials are the isolated bioactive materials produced by the priming methods using UV light plus hypoxic conditions disclosed herein. In some embodiments, the composition is cryopreserved or lyophilized.

In still another aspect of the disclosure, disclosed is a composition comprising the spent culture medium and/or the one or more isolated bioactive materials produced by the priming methods using UV light plus hypoxic conditions disclosed herein. In some embodiments, the composition further comprises a pharmaceutically acceptable carrier which can be a suspension, solution, gel, paste, emulsion, cream, or powder. In some embodiments, the pharmaceutically acceptable carrier comprises one or more of a saline solution, a buffer solution, a sugar, trehalose, a protein, or a starch. In other embodiments, the composition is cryopreserved or lyophilized.

In another aspect of the disclosure, disclosed is a method of regenerating, replacing, or repairing diseased, damaged, or injured body tissue of a subject, the method comprising administering to the subject any of the compositions disclosed herein. In some embodiments, the composition comprises hypoxia primed viable placental tissue. In some embodiments, the composition comprises UV light primed viable placental tissue. In some embodiments, the composition comprises bioactive material primed viable placental tissue. In some embodiments, the composition comprises UV light plus hypoxia primed viable placental tissue. In some embodiments the diseased, damaged or injured body tissue comprises tendons, cartilage, ligaments, periosteum, perichondrium, synovium, fascia, mesentery, sinew, dental tissue, gums, fistulas, nasal septum, vaginal wall tissue, abdominal wall tissue, peritoneum, tumor resection sites, dermal tissue, dermal wounds, dermal lesions, dermal abrasions, dermal burns, first degree burns, partial thickness burns, full thickness burns, epidermal wounds, congenital wounds, toxic epidermal necrolysis, epidermolysis bullosa, pyoderma gangrenosum, dermal ulcers, diabetic ulcers, diabetic foot ulcers, venous ulcers, venous let ulcers, pressure ulcers, arterial ulcers, decubitus ulcers, stasis ulcers, ischemic ulcers, chronic wounds, acute wounds, surgical wounds, internal wounds, hernia, bladder tissue, keloids, adhesions, organ lacerations, organ defects, diseased organ tissue, epithelial defects, spinal cord tissue, nerve tissue, tympanic membranes, and/or mucous membranes. In some embodiments, the compositions are administered topically, subcutaneously, surgically, or by injection, e.g., intramuscular injection. In other embodiments, the compositions are administered by coating the composition onto the surface of a medical device implant and implanting the coated device into the subject.

In another aspect of the disclosure, disclosed is a method of preparing a primed viable placental tissue composition, the method comprising (a) providing the viable placental tissue, wherein the viable placental tissue comprises viable cells native to the viable placental tissue; (b) contacting the viable placental tissue with a culture medium at hypoxic conditions for 24 to 72 hours, thereby preparing primed viable placental tissue; and (c) mincing the primed viable placental tissue to prepare the primed viable placental tissue composition. In some embodiments, the viable placental tissue of step (b) comprises viable umbilical cord tissue, viable amnion membrane tissue, and viable chorion membrane tissue. In some embodiments, the primed viable placental tissue composition of step (c) comprises minced primed viable umbilical cord tissue, minced primed viable amnion membrane tissue, and minced primed viable chorion membrane tissue. In some embodiments the method further comprises lyophilizing the primed viable placental tissue composition. In other embodiments, the method further comprises reconstituting the primed viable placental tissue composition after lyophilization.

Also disclosed in the context of the present invention are the following embodiments 1 to 198. Embodiment 1: A method for priming viable placental tissue, the method comprising: (a) providing the viable placental tissue, wherein the viable placental tissue comprises viable cells native to the viable placental tissue, (b) contacting the viable placental tissue with a culture medium at hypoxic conditions, thereby priming the viable placental tissue and generating spent culture medium, and (c) optionally, separating the primed viable placental tissue from the spent culture medium. Embodiment 2: The method of embodiment 1, wherein the hypoxic conditions are from about 1% to about 5% 02, or from about 1% to about 3% 02, or about 2% 02. Embodiment 3: The method of any one of embodiments 1 or 2, wherein step 1 (b) is conducted for from about 4 to about 96 hours. Embodiment 4: The method of any one of embodiments 1 to 3, wherein step 1 (b) is conducted in a hypoxia chamber or a hypoxia incubator. Embodiment 5: The method of any one of embodiments 1 to 4, wherein the viable placental tissue is amnion tissue, chorion tissue, or umbilical cord tissue, or mixtures thereof Embodiment 6: The method of any one of embodiments 1 to 5, wherein the viable cells comprise one or more of mesenchymal stromal cells (MSCs), epithelial cells, or fibroblasts. Embodiment 7: The method of any one of embodiments 1 to 6, wherein the viable placental tissue is in the form of a sheet, wrap, or graft. Embodiment 8: The method of any one of embodiments 1 to 6, wherein the viable placental tissue is in the form of minced pieces or a powder. Embodiment 9: The method of any one of embodiments 1 to 8, wherein the culture medium comprises a chemically defined culture medium. Embodiment 10: The method of any one of embodiments 1 to 9, wherein the culture medium comprises nutrients. Embodiment 11: The method of any one of embodiments 1 to 10, wherein the culture medium comprises lysed platelets or serum. Embodiment 12: The method of embodiment 11, wherein the serum is fetal bovine serum (FBS) or human serum albumin. Embodiment 13: The method of embodiment 11, wherein the culture medium comprises lysed platelets. Embodiment 14: The method of any one of embodiments 1 to 13, wherein the culture medium comprises DMEM. Embodiment 15: The method of any one of embodiments 1 to 14, further comprising adding an effective amount of one or more bioactive materials to the culture medium in step 1(b). Embodiment 16: The method of embodiment 15, wherein the one or more added bioactive materials comprise growth factors, peptides, antimicrobial peptides, cytokines, enzymes, extracellular vesicles, extracellular matrices, TNF-alpha (preferably at an amount from about 5 to about 50 ng/ml), interferon-gamma (preferably at an amount from about 5 to about 50 ng/ml), or nanoparticles thereof. Embodiment 17: The method of any one of embodiments 1 to 16, wherein the primed viable placental tissue exhibits enhanced angiogenic factor (VEGF-A) function (preferably about a 3 fold to about a 5 fold increase of activity) as determined by a cellular functional assay, an increase in VEGF-A secretion (preferably about a 1 fold to about a 3 fold increase), and/or an increase in IL-1RA secretion (preferably about a 1 fold to about a 3 fold increase) as compared to non-primed viable placental tissue cultured under normoxia conditions. Embodiment 18: The method of any one of embodiments 1 to 17, further comprising collecting the spent culture medium of step 1 (c). Embodiment 19: The method of embodiment 18, further comprising isolating one or more bioactive materials from the spent culture medium. Embodiment 20: The method of embodiment 19, wherein the one or more isolated bioactive materials comprise extracellular vesicles, exosomes, microvesicles, secretomes, cytokines, growth factors, peptides, antimicrobial peptides, or extracellular matrices. Embodiment 21: A composition comprising the primed viable placental tissue produced by the method of any one of embodiments 1 to 17. Embodiment 22: The composition of embodiment 21, wherein the primed viable placental tissue is in the form of a sheet, wrap, or graft. Embodiment 23: The composition of embodiment 21, wherein the primed viable placental tissue is in the form of minced pieces or a powder. Embodiment 24: The composition of embodiment 23, wherein the composition further comprises a pharmaceutically acceptable carrier. Embodiment 25: The composition of embodiment 24, wherein the pharmaceutically acceptable carrier is a suspension, solution, gel, paste, emulsion, cream, or powder. Embodiment 26: The composition of any one of embodiments 24 or 25, wherein the pharmaceutically acceptable carrier comprises one or more of a saline solution, a buffer solution, a sugar, trehalose, a protein, or a starch. Embodiment 27: The composition of any one of embodiments 21 to 26, wherein the composition is cryopreserved or lyophilized. Embodiment 28: A composition comprising hypoxia primed viable placental tissue. Embodiment 29: The composition of embodiment 28, wherein the hypoxia primed viable placental tissue exhibits enhanced angiogenic factor (VEGF-A) function (preferably about a 3 fold to about a 5 fold increase of activity) as determined by a cellular functional assay, an increase in VEGF-A secretion (preferably about a 1 fold to about a 3 fold increase), and/or an increase in IL-1RA secretion (preferably about a 1 fold to about a 3 fold increase) as compared to non-primed viable placental tissue cultured under normoxia conditions. Embodiment 30: The composition of any one of embodiments 28 or 29, wherein the hypoxia primed viable placental tissue is amnion tissue, chorion tissue, or umbilical cord tissue, or mixtures thereof. Embodiment 31: The composition of any one of embodiments 28 to 30, wherein the hypoxia primed viable placental tissue comprises one or more of MSCs, epithelial cells, or fibroblasts. Embodiment 32: The composition of any one of embodiments 28 to 31, wherein the hypoxia primed viable placental tissue is in the form of a sheet, wrap, or graft. Embodiment 33: The composition any one of embodiments 28 to 31, wherein the hypoxia primed viable placental tissue is in the form of minced pieces or a powder. Embodiment 34: The composition of embodiment 33, wherein the composition further comprises a pharmaceutically acceptable carrier. Embodiment 35: The composition of embodiment 34, wherein the pharmaceutically acceptable carrier is a suspension, solution, gel, paste, emulsion, cream, or powder. Embodiment 36: The composition of anyone of embodiments 34 or 35, wherein the pharmaceutically acceptable carrier comprises one or more of a saline solution, a buffer solution, a sugar, trehalose, a protein, or a starch. Embodiment 37: The composition of any one of embodiments 28 to 36, wherein the composition further comprises one or more bioactive materials. Embodiment 38: The composition of embodiment 37, wherein the one or more bioactive materials comprise extracellular vesicles, exosomes, microvesicles, secretomes, cytokines, growth factors, peptides, antimicrobial peptides, extracellular matrices, TNF-alpha (preferably at an amount from about 5 to about 50 ng/ml), interferon-gamma (preferably at an amount from about 5 to about 50 ng/ml), or nanoparticles thereof. Embodiment 39: The composition of any one of embodiments 37 or 38, wherein the one or more bioactive materials are the isolated bioactive materials of the method of any one of embodiments 19 or 20. Embodiment 40: The composition of any one of embodiments 28 to 39, wherein the composition is cryopreserved or lyophilized. Embodiment 41: A composition comprising the spent culture medium of the method of embodiment 18 and/or the one or more isolated bioactive materials of the method of any one of embodiments 19 or 20. Embodiment 42: The composition of embodiment 41, wherein the composition further comprises a pharmaceutically acceptable carrier. Embodiment 43: The composition of embodiment 42, wherein the pharmaceutically acceptable carrier is a suspension, solution, gel, paste, emulsion, cream, or powder. Embodiment 44: The composition of any one of embodiments 42 or 43, wherein the pharmaceutically acceptable carrier comprises one or more of a saline solution, a buffer solution, a sugar, trehalose, a protein, or a starch. Embodiment 45: A method of regenerating, replacing, or repairing diseased, damaged, or injured body tissue of a subject, the method comprising administering to the subject the composition of any one of embodiments 19 to 44. Embodiment 46: The method of embodiment 45, wherein the diseased, damaged or injured body tissue comprises tendons, cartilage, ligaments, periosteum, perichondrium, synovium, fascia, mesentery, sinew, dental tissue, gums, fistulas, nasal septum, vaginal wall tissue, abdominal wall tissue, peritoneum, tumor resection sites, dermal tissue, dermal wounds, dermal lesions, dermal abrasions, dermal burns, first degree burns, partial thickness burns, full thickness burns, epidermal wounds, congenital wounds, toxic epidermal necrolysis, epidermolysis bullosa, pyoderma gangrenosum, dermal ulcers, diabetic ulcers, diabetic foot ulcers, venous ulcers, venous let ulcers, pressure ulcers, arterial ulcers, decubitus ulcers, stasis ulcers, ischemic ulcers, chronic wounds, acute wounds, surgical wounds, internal wounds, hernia, bladder tissue, keloids, adhesions, organ lacerations, organ defects, diseased organ tissue, epithelial defects, spinal cord tissue, nerve tissue, tympanic membranes, and/or mucous membranes. Embodiment 47: The method of any one of embodiments 45 or 46, wherein the composition is administered topically, subcutaneously, surgically, or by injection. Embodiment 48: The method of any one of embodiments 45 or 46, wherein the composition is administered by coating the composition onto the surface of a medical device implant and implanting the coated device into the subject. Embodiment 49: A method for priming viable placental tissue, the method comprising: (a) providing the viable placental tissue, wherein the viable placental tissue comprises viable cells native to the viable placental tissue, and (b) exposing the viable placental tissue to UV light, thereby priming the viable placental tissue. Embodiment 50: The method of embodiment 49, wherein the UV light is UVA light, or UVB light, or combinations thereof. Embodiment 51: The method of any one of embodiments 49 or 50, wherein the UV light is UVB light. Embodiment 52: The method of any one of embodiments 49 to 51, wherein the UV light exposure time is from about 10 seconds to about 4 minutes, or from about 20 seconds to about 180 seconds. Embodiment 53: The method of any one of embodiments 49 to 52, wherein the viable placental tissue is amnion tissue, chorion tissue, or umbilical cord tissue, or mixtures thereof. Embodiment 54: The method of any one of embodiments 49 to 53, wherein the viable cells comprise one or more of mesenchymal stromal cells (MSCs), epithelial cells, or fibroblasts. Embodiment 55: The method of any one of embodiments 49 to 54, wherein the viable placental tissue is in the form of a sheet, wrap, or graft. Embodiment 56: The method of any one of embodiments 49 to 54, wherein the viable placental tissue is in the form of minced pieces or a powder. Embodiment 57: The method of any one of embodiments 49 to 56, further comprising: (c) contacting the primed viable placental tissue with a culture medium and thereby generating spent culture medium, and (d) optionally, separating the primed viable placental tissue from the spent culture medium. Embodiment 58: The method of embodiment 57, wherein step 57 (c) is conducted from about 4 to about 96 hours, or from about 24 to about 96 hours. Embodiment 59: The method of any one of embodiments 57 or 58, wherein step 55 (c) is conducted in an incubator. Embodiment 60: The method of any one of embodiments 49 to 59, wherein the culture medium comprises a chemically defined culture medium. Embodiment 61: The method of any one of embodiments 57 to 60, wherein the culture medium comprises nutrients. Embodiment 62: The method of any one of embodiments 57 to 61, wherein the culture medium comprises lysed platelets or serum. Embodiment 63: The method of embodiment 62, wherein the serum is fetal bovine serum (FBS) or human serum albumin. Embodiment 64: The method of embodiment 62, wherein the culture medium comprises lysed platelets. Embodiment 65: The method of any one of embodiments 57 to 64, wherein the culture medium comprises DMEM. Embodiment 66: The method of any one of embodiments 57 to 65, further comprising adding an effective amount of one or more bioactive materials to the culture medium in step 55 (c). Embodiment 67: The method of embodiment 66, wherein the added one or more bioactive materials comprise growth factors, peptides, antimicrobial peptides, cytokines, enzymes, extracellular vesicles, extracellular matrices, TNF-alpha (preferably at an amount from about 5 to about 50 ng/ml), interferon-gamma (preferably at an amount from about 5 to about 50 ng/ml), or nanoparticles thereof. Embodiment 68: The method of any one of embodiments 49 to 67, wherein the primed viable placental tissue exhibits an increased HBD-2 secretion (preferably about a 1 fold to about a 4 fold increase) as compared to viable placental tissue not exposed to UV light (non-primed). Embodiment 69: The method of any one of embodiments 57 to 68, further comprising collecting the spent culture medium of step 57 (d). Embodiment 70: The method of embodiment 69, further comprising isolating one or more bioactive materials from the spent culture medium. Embodiment 71: The method of embodiment 70, wherein the isolated one or more bioactive materials comprise extracellular vesicles, exosomes, microvesicles, secretomes, cytokines, growth factors, peptides, antimicrobial peptides, or extracellular matrices. Embodiment 72: A composition comprising the primed viable placental tissue produced by the method of any one of embodiments 49 to 68. Embodiment 73: The composition of embodiment 72, wherein the primed viable placental tissue is in the form of a sheet, wrap, or graft. Embodiment 74: The composition of embodiment 72, wherein the primed viable placental tissue is in the form of minced pieces or a powder. Embodiment 75: The composition of embodiment 74, wherein the composition further comprises a pharmaceutically acceptable carrier. Embodiment 76: The composition of embodiment 75, wherein the pharmaceutically acceptable carrier is a suspension, solution, gel, paste, emulsion, cream, or powder. Embodiment 77: The composition of anyone of embodiments 75 or 76, wherein the pharmaceutically acceptable carrier comprises one or more of a saline solution, a buffer solution, a sugar, trehalose, a protein, or a starch. Embodiment 78: The composition of any one of embodiments 72 to 77, wherein the composition is cryopreserved or lyophilized. Embodiment 79: A composition comprising UV light primed viable placental tissue. Embodiment 80: The composition of embodiment 79, wherein the UV light primed placental tissue exhibits an increased HBD-2 secretion (preferably about a 1 fold to about a 4 fold increase) as compared to viable placental tissue not exposed to UV light (non-primed). Embodiment 81: The composition of any one of embodiments 79 or 80, wherein the UV light primed viable placental tissue is amnion tissue, chorion tissue, or umbilical cord tissue, or mixtures thereof. Embodiment 82: The composition of any one of embodiments 79 to 81, wherein the UV light primed viable placental tissue comprises one or more of MSCs, epithelial cells, or fibroblasts. Embodiment 83: The composition of any one of embodiments 79 to 82, wherein the UV light primed viable placental tissue is in the form of a sheet, wrap, or graft. Embodiment 84: The composition any one of embodiments 79 to 82, wherein the UV light primed viable placental tissue is in the form of minced pieces or a powder. Embodiment 85: The composition of embodiment 84, wherein the composition further comprises a pharmaceutically acceptable carrier. Embodiment 86: The composition of embodiment 85, wherein the pharmaceutically acceptable carrier is a suspension, solution, gel, paste, emulsion, cream, or powder. Embodiment 87: The composition of anyone of embodiments 85 or 86, wherein the pharmaceutically acceptable carrier comprises one or more of a saline solution, a buffer solution, a sugar, trehalose, a protein, or a starch. Embodiment 88: The composition of any one of embodiments 79 to 87, wherein the composition further comprises one or more bioactive materials. Embodiment 89: The composition of embodiment 88, wherein the one or more bioactive materials comprise extracellular vesicles, exosomes, microvesicles, secretomes, cytokines, growth factors, peptides, antimicrobial peptides, extracellular matrices, TNF-alpha (preferably at an amount from about 5 to about 50 ng/ml), interferon-gamma (preferably at an amount from 5 to 50 ng/ml), or nanoparticles thereof. Embodiment 90: The composition of any one of embodiments 88 or 89, wherein the one or more bioactive materials are the isolated bioactive materials of the method of any one of embodiments 70 or 71. Embodiment 91: The composition of any one of embodiments 79 to 90, wherein the composition is cryopreserved or lyophilized. Embodiment 92: A composition comprising the spent culture medium of the method of embodiment 69 and/or the one or more isolated bioactive materials produced by the method of any one of embodiments 70 or 71. Embodiment 93: The composition of embodiment 92, wherein the composition further comprises a pharmaceutically acceptable carrier. Embodiment 94: The composition of embodiment 93, wherein the pharmaceutically acceptable carrier is a suspension, solution, gel, paste, emulsion, cream, or powder. Embodiment 95: The composition of any one of embodiments 93 or 94, wherein the pharmaceutically acceptable carrier comprises one or more of a saline solution, a buffer solution, a sugar, trehalose, a protein, or a starch. Embodiment 96: A method of regenerating, replacing, or repairing diseased, damaged, or injured body tissue of a subject, the method comprising administering to the subject the composition of any one of embodiments 72 to 95. Embodiment 97: The method of embodiment 96, wherein the diseased, damaged or injured body tissue comprises tendons, cartilage, ligaments, periosteum, perichondrium, synovium, fascia, mesentery, sinew, dental tissue, gums, fistulas, nasal septum, vaginal wall tissue, abdominal wall tissue, peritoneum, tumor resection sites, dermal tissue, dermal wounds, dermal lesions, dermal abrasions, dermal burns, first degree burns, partial thickness burns, full thickness burns, epidermal wounds, congenital wounds, toxic epidermal necrolysis, epidermolysis bullosa, pyoderma gangrenosum, dermal ulcers, diabetic ulcers, diabetic foot ulcers, venous ulcers, venous let ulcers, pressure ulcers, arterial ulcers, decubitus ulcers, stasis ulcers, ischemic ulcers, chronic wounds, acute wounds, surgical wounds, internal wounds, hernia, bladder tissue, keloids, adhesions, organ lacerations, organ defects, diseased organ tissue, epithelial defects, spinal cord tissue, nerve tissue, tympanic membranes, and/or mucous membranes. Embodiment 98: The method of any one of embodiments 96 or 97, wherein the composition is administered topically, subcutaneously, surgically, or by injection. Embodiment 99: The method of any one of embodiments 96 or 97, wherein the composition is administered by coating the composition onto the surface of a medical device implant and implanting the coated device into the subject. Embodiment 100: A method for priming viable placental tissue, the method comprising: (a) providing the viable placental tissue, wherein the viable placental tissue comprises viable cells native to the viable placental tissue, (b) contacting the tissue with a culture medium comprising an effective amount of one or more bioactive materials, thereby priming the viable placental tissue and generating spent culture medium, and (c) optionally, separating the primed viable placental tissue from the spent culture medium. Embodiment 101: The method of embodiment 100, wherein the one or more bioactive materials comprise growth factors, peptides, antimicrobial peptides, cytokines, enzymes, extracellular vesicles, extracellular matrices, TNF-alpha (preferably at an amount from about 5 to about 50 ng/ml), interferon-gamma (preferably at an amount of from about 5 to about 50 ng/ml), or nanoparticles thereof. Embodiment 102: The method of any one of embodiments 100 or 101, wherein step 100 (b) is conducted from about 4 to about 96 hours. Embodiment 103: The method of any one of embodiments 100 to 102, wherein step 100 (b) is conducted in an incubator. Embodiment 104: The method of any one of embodiments 100 to 103, wherein the viable placental tissue is amnion tissue, chorion tissue, or umbilical cord tissue, or mixtures thereof. Embodiment 105: The method of any one of embodiments 100 to 104, wherein the viable cells comprise one or more of mesenchymal stromal cells (MSCs), epithelial cells, or fibroblasts. Embodiment 106: The method of any one of embodiments 100 to 105, wherein the viable placental tissue is in the form of a sheet, wrap, or graft. Embodiment 107: The method of any one of embodiments 100 to 105, wherein the viable placental tissue is in the form of minced pieces or a powder. Embodiment 108: The method of any one of embodiments 100 to 107, wherein the culture medium further comprises a chemically defined culture medium. Embodiment 109: The method of any one of embodiments 100 to 108, wherein the culture medium further comprises nutrients. Embodiment 110: The method of any one of embodiments 100 to 109, wherein the culture medium further comprises lysed platelets or serum. Embodiment 111: The method of embodiment 110, wherein the serum is fetal bovine serum (FBS) or human serum albumin. Embodiment 112: The method of embodiment 110, wherein the culture medium further comprises lysed platelets. Embodiment 113: The method of any one of embodiments 100 to 112, wherein the culture medium further comprises DMEM. Embodiment 114: The method of any one of embodiment 100 to 113, wherein the primed viable placental tissue exhibits one or more increased tissue regenerative properties comprising angiogenesis, anti-inflammatory, chemoattractant, antimicrobial, antioxidant or antifibrosis as compared to non-primed viable placental tissue as determined in vitro such as with ELISA and/or Multi-plex analysis, and/or in vivo. Embodiment 115: The method of any one of embodiments 100 to 114, further comprising collecting the spent culture medium of step 100 (c). Embodiment 116: The method of embodiment 115, further comprising isolating one or more bioactive materials from the spent culture medium. Embodiment 117: The method of embodiment 116, wherein the isolated one or more bioactive materials comprise extracellular vesicles, exosomes, microvesicles, secretomes, cytokines, growth factors, peptides, antimicrobial peptides, or extracellular matrices. Embodiment 118: A composition comprising the primed viable placental tissue produced by the method of any one of embodiments 100 to 117. Embodiment 119: The composition of embodiment 118, wherein the primed viable placental tissue is in the form of a sheet, wrap, or graft. Embodiment 120: The composition of embodiment 118, wherein the primed viable placental tissue is in the form of minced pieces or a powder. Embodiment 121: The composition of embodiment 120, wherein the composition further comprises a pharmaceutically acceptable carrier. Embodiment 122: The composition of embodiment 121, wherein the pharmaceutically acceptable carrier is a suspension, solution, gel, paste, emulsion, cream, or powder. Embodiment 123: The composition of any one of embodiments 121 or 122, wherein the pharmaceutically acceptable carrier comprises one or more of a saline solution, a buffer solution, a sugar, trehalose, a protein, or a starch. Embodiment 124: The composition of any one of embodiments 118 to 123, wherein the composition is cryopreserved or lyophilized. Embodiment 125: A composition comprising bioactive material primed viable placental tissue. Embodiment 126: The composition of embodiment 125, wherein the bioactive material primed viable placental tissue exhibits one or more increased tissue regenerative properties comprising angiogenesis, anti-inflammatory, chemoattractant, antimicrobial, antioxidant or antifibrosis as compared to non-primed viable placental tissue as determined in vitro such as with ELISA and/or Multi-plex analysis, and/or in vivo. Embodiment 127: The composition of any one of embodiments 125 or 126, wherein the bioactive material viable placental tissue is amnion tissue, chorion tissue, or umbilical cord tissue, or mixtures thereof. Embodiment 128: The composition of any one of embodiments 125 to 127, wherein the bioactive material primed viable placental tissue comprises one or more of MSCs, epithelial cells, or fibroblasts. Embodiment 129: The composition of any one of embodiments 125 to 128, wherein the bioactive material primed viable placental tissue is in the form of a sheet, wrap, or graft. Embodiment 130: The composition any one of embodiments 125 to 128, wherein the bioactive material primed viable placental tissue is in the form of minced pieces or a powder. Embodiment 131: The composition of embodiment 130, wherein the composition further comprises a pharmaceutically acceptable carrier. Embodiment 132: The composition of embodiment 131, wherein the pharmaceutically acceptable carrier is a suspension, solution, gel, paste, emulsion, cream, or powder. Embodiment 133: The composition of anyone of embodiments 131 or 132, wherein the pharmaceutically acceptable carrier comprises one or more of a saline solution, a buffer solution, a sugar, trehalose, a protein, or a starch. Embodiment 134: The composition of any one of embodiments 125 to 133, wherein the composition further comprises one or more bioactive materials. Embodiment 135: The composition of embodiment 134, wherein the one or more bioactive materials comprise extracellular vesicles, exosomes, microvesicles, secretomes, cytokines, growth factors, peptides, antimicrobial peptides, extracellular matrices, TNF-alpha (preferably at an amount from about 5 to about 50 ng/ml), interferon-gamma (preferably at an amount from about 5 to about 50 ng/ml), or nanoparticles thereof. Embodiment 136: The composition of any one of embodiments 134 or 135, wherein the one or more bioactive materials are the isolated bioactive materials of the method of any one of embodiments 116 or 117. Embodiment 137: The composition of any one of embodiments 125 to 136, wherein the composition is cryopreserved or lyophilized. Embodiment 138: A composition comprising the spent culture medium of the method of embodiment 115 and/or the one or more isolated bioactive materials produced by the method of any one of embodiments 116 or 117. Embodiment 139: The composition of embodiment 138, wherein the composition further comprises a pharmaceutically acceptable carrier. Embodiment 140: The composition of embodiment 139, wherein the pharmaceutically acceptable carrier is a suspension, solution, gel, paste, emulsion, cream, or powder. Embodiment 141: The composition of any one of embodiments 139 or 140, wherein the pharmaceutically acceptable carrier comprises one or more of a saline solution, a buffer solution, a sugar, trehalose, a protein, or a starch. Embodiment 142: A method of regenerating, replacing, or repairing diseased, damaged, or injured body tissue of a subject, the method comprising administering to the subject the composition of any one of embodiments 118 to 141. Embodiment 143: The method of embodiment 142, wherein the diseased, damaged or injured body tissue comprises tendons, cartilage, ligaments, periosteum, perichondrium, synovium, fascia, mesentery, sinew, dental tissue, gums, fistulas, nasal septum, vaginal wall tissue, abdominal wall tissue, peritoneum, tumor resection sites, dermal tissue, dermal wounds, dermal lesions, dermal abrasions, dermal burns, first degree burns, partial thickness burns, full thickness burns, epidermal wounds, congenital wounds, toxic epidermal necrolysis, epidermolysis bullosa, pyoderma gangrenosum, dermal ulcers, diabetic ulcers, diabetic foot ulcers, venous ulcers, venous let ulcers, pressure ulcers, arterial ulcers, decubitus ulcers, stasis ulcers, ischemic ulcers, chronic wounds, acute wounds, surgical wounds, internal wounds, hernia, bladder tissue, keloids, adhesions, organ lacerations, organ defects, diseased organ tissue, epithelial defects, spinal cord tissue, nerve tissue, tympanic membranes, and/or mucous membranes. Embodiment 144: The method of any one of embodiments 142 or 143, wherein the composition is administered topically, subcutaneously, surgically, or by injection. Embodiment 145: The method of any one of embodiments 142 or 143, wherein the composition is administered by coating the composition onto the surface of a medical device implant and implanting the coated device into the subject. Embodiment 146: A method for priming viable placental tissue, the method comprising: (a) providing the viable placental tissue, wherein the viable placental tissue comprises viable cells native to the viable placental tissue, (b) exposing the viable placental tissue to UV light, (c) contacting the viable placental tissue with a culture medium at hypoxic conditions, thereby priming the viable placental tissue and generating spent culture medium, and (d) optionally, separating the primed viable placental tissue from the spent culture medium. Embodiment 147: The method of embodiment 146, wherein the UV light is UV-A light, or UV-B light, or combinations thereof. Embodiment 148: The method of any one of embodiments 146 or 147, wherein the hypoxic conditions are from about 1% to about 5% 02 or from about 1% to about 3% 02, or about 2% 02. Embodiment 149: The method of any one of embodiments 146 to 148, wherein step 1 (c) is conducted from about 4 to about 96 hours. Embodiment 150: The method of any one of embodiments 146 to 149, wherein step 146 (c) is conducted in a hypoxia chamber or a hypoxia incubator. Embodiment 151: The method of any one of embodiments 146 to 150, wherein the viable placental tissue is amnion tissue, chorion tissue, or umbilical cord tissue, or mixtures thereof. Embodiment 152: The method of any one of embodiments 146 to 151, wherein the viable cells comprise one or more of mesenchymal stromal cells (MSCs), epithelial cells, or fibroblasts. Embodiment 153: The method of any one of embodiments 146 to 152, wherein the viable placental tissue is in the form of a sheet, wrap, or graft. Embodiment 154: The method of any one of embodiments 146 to 152, wherein the viable placental tissue is in the form of minced pieces or a powder. Embodiment 155: The method of any one of embodiments 146 to 154, wherein the culture medium comprises a chemically defined culture medium. Embodiment 156: The method of any one of embodiments 146 to 155, wherein the culture medium comprises nutrients. Embodiment 157: The method of any one of embodiments 146 to 156, wherein the culture medium comprises lysed platelets or serum. Embodiment 158: The method of embodiment 157, wherein the serum is fetal bovine serum (FBS) or human serum albumin. Embodiment 159: The method of embodiment 157, wherein the culture medium comprises lysed platelets. Embodiment 160: The method of any one of embodiments 146 to 159, wherein the culture medium comprises DMEM. Embodiment 161: The method of any one of embodiments 146 to 160, further comprises adding an effective amount of one or more bioactive materials to the culture medium in step 146 (c). Embodiment 162: The method of embodiment 161, wherein the one or more added bioactive materials comprise growth factors, peptides, antimicrobial peptides, cytokines, enzymes, extracellular vesicles, extracellular matrices, TNF-alpha (preferably at an amount from about 5 to about 50 ng/ml), interferon-gamma (preferably at an amount from about 5 to about 50 ng/ml), or nanoparticles thereof. Embodiment 163: The method of any one of embodiments 146 to 162, wherein the primed viable placental tissue exhibits one or more increased tissue regenerative properties comprising an increase in HBD-2 secretion (preferably about a 1 fold to about a 4 fold increase); enhanced angiogenic factor (VEGF-A) function (preferably about a 3 fold to about a 5 fold increase of activity) as determined by a cellular functional assay; an increase in VEGF-A secretion (preferably about a 1 fold to about a 3 fold increase); and/or an increase in IL-1RA secretion (preferably about a 1 fold to about a 3 fold increase) as compared to viable placental tissue not exposed to hypoxic conditions and UV light (non-primed). Embodiment 164: The method of any one of embodiments 146 to 163, further comprising collecting the spent culture medium of step 146 (d). Embodiment 165: The method of embodiment 164, further comprising isolating one or more bioactive materials from the spent culture medium. Embodiment 166: The method of embodiment 165, wherein the isolated one or more bioactive materials comprise extracellular vesicles, exosomes, microvesicles, secretomes, cytokines, growth factors, peptides, antimicrobial peptides, or extracellular matrices. Embodiment 167: A composition comprising the primed viable placental tissue produced by the method of any one of embodiments 146 to 163.

Embodiment 168: The composition of embodiment 167, wherein the primed viable placental tissue is in the form of a sheet, wrap, or graft. Embodiment 169: The composition of embodiment 167, wherein the primed viable placental tissue is in the form of minced pieces or a powder. Embodiment 170: The composition of embodiment 169, wherein the composition further comprises a pharmaceutically acceptable carrier. Embodiment 171: The composition of embodiment 170, wherein the pharmaceutically acceptable carrier is a suspension, solution, gel, paste, emulsion, cream, or powder. Embodiment 172: The composition of anyone of embodiments 170 or 171, wherein the pharmaceutically acceptable carrier comprises one or more of a saline solution, a buffer solution, a sugar, trehalose, a protein, or a starch. Embodiment 173: The composition of any one of embodiments 167 to 172, wherein the composition is cryopreserved or lyophilized. Embodiment 174: A composition comprising UV light plus hypoxia primed viable placental tissue. Embodiment 175: The composition of embodiment 174, wherein the UV light plus hypoxia primed viable placental tissue exhibits one or more increased tissue regenerative properties comprising an increase of HBD-2 secretion (preferably about a 1 fold to about a 4 fold increase); enhanced angiogenic factor (VEGF-A) function (preferably about a 3 fold to about a 5 fold increase of activity) as determined by a cellular functional assay; an increase in VEGF-A secretion (preferably about a 1 fold to about a 3 fold increase); and/or an increase in IL-1RA secretion (preferably about a 1 fold to about a 3 fold increase) as compared to viable placental tissue not exposed to hypoxic conditions and UV light (non-primed). Embodiment 176: The composition of any one of embodiments 174 or 175, wherein the UV light plus hypoxia primed viable placental tissue is amnion tissue, chorion tissue, or umbilical cord tissue, or mixtures thereof. Embodiment 177: The composition of any one of embodiments 174 to 176, wherein the UV light plus hypoxia primed viable placental tissue is in the form of a sheet, wrap, or graft. Embodiment 178: The composition any one of embodiments 174 to 176, wherein the UV light plus hypoxia primed viable placental tissue is in the form of minced pieces or a powder. Embodiment 179: The composition of embodiment 178, wherein the composition further comprises a pharmaceutically acceptable carrier. Embodiment 180: The composition of embodiment 179, wherein the pharmaceutically acceptable carrier is a suspension, solution, gel, paste, emulsion, cream, or powder. Embodiment 181: The composition of anyone of embodiments 179 or 180, wherein the pharmaceutically acceptable carrier comprises one or more of a saline solution, a buffer solution, a sugar, trehalose, a protein, or a starch. Embodiment 182: The composition of any one of embodiments 174 to 181, wherein the composition further comprises one or more bioactive materials. Embodiment 183: The composition of embodiment 182, wherein the one or more bioactive materials comprise extracellular vesicles, exosomes, microvesicles, secretomes, cytokines, growth factors, peptides, antimicrobial peptides, extracellular matrices, TNF-alpha (preferably at an amount from about 5 to about 50 ng/ml), interferon-gamma (preferably at an amount from about 5 to about 50 ng/ml), or nanoparticles thereof. Embodiment 184: The composition of any one of embodiments 182 or 183, wherein the one or more bioactive materials are the isolated bioactive materials of the method of any one of embodiments 165 or 166. Embodiment 185: The composition of any one of embodiments 174 to 184, wherein the composition is cryopreserved or lyophilized. Embodiment 186: A composition comprising the spent culture medium of the method of embodiment 164 and/or the one or more isolated bioactive materials produced by the method of any one of embodiments 165 or 166. Embodiment 187: The composition of embodiment 186, wherein the composition further comprises a pharmaceutically acceptable carrier. Embodiment 188: The composition of embodiment 187, wherein the pharmaceutically acceptable carrier is a suspension, solution, gel, paste, emulsion, cream, or powder. Embodiment 189: The composition of any one of embodiments 187 or 188, wherein the pharmaceutically acceptable carrier comprises one or more of a saline solution, a buffer solution, a sugar, trehalose, a protein, or a starch. Embodiment 190: A method of regenerating, replacing, or repairing diseased, damaged, or injured body tissue of a subject, the method comprising administering to the subject the composition of any one of embodiments 167 to 189. Embodiment 191: The method of embodiment 190, wherein the diseased, damaged or injured body tissue comprises tendons, cartilage, ligaments, periosteum, perichondrium, synovium, fascia, mesentery, sinew, dental tissue, gums, fistulas, nasal septum, vaginal wall tissue, abdominal wall tissue, peritoneum, tumor resection sites, dermal tissue, dermal wounds, dermal lesions, dermal abrasions, dermal burns, first degree burns, partial thickness burns, full thickness burns, epidermal wounds, congenital wounds, toxic epidermal necrolysis, epidermolysis bullosa, pyoderma gangrenosum, dermal ulcers, diabetic ulcers, diabetic foot ulcers, venous ulcers, venous let ulcers, pressure ulcers, arterial ulcers, decubitus ulcers, stasis ulcers, ischemic ulcers, chronic wounds, acute wounds, surgical wounds, internal wounds, hernia, bladder tissue, keloids, adhesions, organ lacerations, organ defects, diseased organ tissue, epithelial defects, spinal cord tissue, nerve tissue, tympanic membranes, and/or mucous membranes. Embodiment 192: The method of any one of embodiments 190 or 191, wherein the composition is administered topically, subcutaneously, surgically, or by injection. Embodiment 193: The method of any one of embodiments 190 or 191, wherein the composition is administered by coating the composition onto the surface of a medical device implant and implanting the coated device into the subject. Embodiment 194: A method of preparing a primed viable placental tissue composition, the method comprising: (a) providing the viable placental tissue, wherein the viable placental tissue comprises viable cells native to the viable placental tissue; (b) contacting the viable placental tissue with a culture medium at hypoxic conditions for 24 to 72 hours, thereby preparing primed viable placental tissue; and (c) mincing the primed viable placental tissue to prepare the primed viable placental tissue composition. Embodiment 195: The method of embodiment 194, wherein the viable placental tissue of step (b) comprises viable umbilical cord tissue, viable amnion membrane tissue, and viable chorion membrane tissue. Embodiment 196: The method of embodiment 194 or 195, wherein the primed viable placental tissue composition of step (c) comprises minced primed viable umbilical cord tissue, minced primed viable amnion membrane tissue, and minced primed viable chorion membrane tissue. Embodiment 197: The method of any one of embodiments 194 to 196, further comprising lyophilizing the primed viable placental tissue composition. Embodiment 198: The method of embodiment 197, further comprising reconstituting the primed viable placental tissue composition after lyophilization.

The terms “priming,” “prime,” or “primed” as used herein means exposure of tissue, e.g., placental tissue, to an external stimulus which evokes an enhancement of the therapeutic properties of the tissue and the cells contained therein.

The term “native cells” as used herein means cells that are native, resident, or endogenous to the placental tissue or that are present in the placental tissue when it is obtained from the donor and have not been removed or isolated from the placental tissue or cultured outside of the placental tissue, i.e., cells that are not exogenously added to the placental tissue.

The term “viable placental tissue” as used herein means placental tissue that contains viable native cells. Isolated cells and cells cultured outside of a tissue are not themselves “viable placental tissue.”

The term “MSCs” as used herein means mesenchymal stromal cells and includes mesenchymal stem cells.

The term “lysed platelets” as used herein means lysed platelets from human origin, also known as “human lysed platelets” or “human platelet lysates.” The terms “spent culture medium,” “spent culture media,” “conditioned medium,” or “conditioned media” as used herein mean culture medium or media that has been used in a culturing process.

The term “bioactive material(s)” as used herein means a substance(s) or a compound(s) having a biological effect upon a living organism, tissue, or cell.

The term “subject” as used herein mean a vertebrate animal. The vertebrate animal can be a mammal. The mammal can be a primate including a human (Homo sapiens). In some embodiments, the subject is a human (Homo sapiens).

The terms “optional” or “optionally” as used herein mean that the subsequently described event, circumstance, or material may or may not occur or be present, and that the description includes instances where the event, circumstance, or material occurs or is present and instances where it does not occur or is not present.

The term “partially depleted” as used herein means a reduction in the amount of a type of cell, a type of tissue, or blood in placental tissue.

The term “substantially free of” as used herein means less than about 0.5%, or less than about 1%, or less than about 5% of a type of cell, a type of tissue, or blood in placental tissue.

The term “free of” as used herein means the complete absence of a type of cell, a type of tissue, or blood in placental tissue.

The terms “about” or “approximately” as used herein are defined as being close to as understood by one of skill in the art, and in one non-limiting embodiment the terms are defined to be within 10%, preferably within 5%, more preferably within 1%, and most preferably within 0.5%.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) as used herein are inclusive or open-ended and do not exclude additional, unrecited elements or method steps.

The use of the word “a” or “an” when used in conjunction with the terms “comprising”, “having”, “including”, or “containing” (or any variations of these words) may mean “one,” but it is also consistent with the meaning of “one or more,” “at least one,” and “one or more than one.”

For purposes of this application, a number value with one or more decimal places can be rounded to the nearest whole number using standard rounding guidelines, i.e. round up if the number being rounded is 5, 6, 7, 8, or 9; and round down if the number being rounded is 0, 1, 2, 3, or 4. For example, 0.42 can be rounded to 0.4.

The compositions and methods for their use can “comprise,” “consist essentially of,” or “consist of” any of the ingredients or steps disclosed throughout the specification.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a chart showing levels of IL-1RA in hypoxia primed amnion tissue vs. normoxia cultured amnion tissue from 3 donors as determined by multi-plex assay. The levels of IL-1RA were quantified based on standard curve. Results from each individual donor and the average of the 3 donors are shown. Error bar=SEM.

FIG. 2 is a chart showing levels of IL-1RA in hypoxia primed chorion tissue vs. normoxia cultured chorion tissue from 3 donors as determined by multi-plex assay. The levels of IL-1RA were quantified based on standard curve. Results from each individual donor and the average of the 3 donors are shown. Error bar=SEM.

FIG. 3 is a chart showing levels of VEGF-A in the spent (conditioned) culture medium of hypoxia primed chorion tissue vs. the spent culture medium of normoxia cultured chorion tissue from 3 donors as determined by multi-plex assay. The levels of VEGF-A were quantified based on standard curve. Results from each individual donor and the average of the 3 donors are shown. Error bar=SEM.

FIG. 4 is a chart showing levels of VEGF-A in the spent (conditioned) culture medium of hypoxia primed chorion tissue vs. the spent culture medium of normoxia cultured chorion tissue from Donor 3 in FIG. 3 as determined by VEGF-A reporter bioassay. The levels of VEGF-A were quantified based on VEGF-A standard curve. Error bar—SEM. P value=0.012.

FIG. 5 is a chart showing levels of HBD-2 in the spent (conditioned) medium of UVB light primed amnion tissue exposed at various times vs. the spent culture medium of non-exposed, non-primed amnion tissue (control) as determined by ELISA.

FIG. 6 is a chart showing levels of HBD-2 in UVB light primed amnion tissue exposed at various times vs. non-exposed, non-primed amnion tissue (control) as determined by ELISA.

FIG. 7 is a chart showing levels of HBD-2 in the spent (conditioned) medium of UVB light primed chorion tissue exposed at various times vs. the spent culture medium of non-exposed, non-primed chorion tissue (control) as determined by ELISA.

FIG. 8 is a chart showing levels of HBD-2 in UVB light primed chorion tissue exposed at various times vs. non-exposed, non-primed chorion tissue (control) as determined by ELISA.

FIG. 9A is a photo image of a bipedicle flap on a rat's back.

FIG. 9B is a doppler image of a bipedicle flap on a rat's back.

FIG. 10 is a photograph of wounds taken over a 28-day period post-wounding in control and hypoxia primed viable placental tissue mixture (FPF) treated groups.

FIG. 11 is a graph showing wound closure versus days post wounding expressed as a percent of the wound area at Day 0 for hypoxia primed viable placental tissue mixture (FPF) treated groups and control. Mean—SD for 5 rats per treatment group are shown for each time point on the graphs. N=10, ns: not significant, *p<0.05, ***p<0.005, ****p<0.001.

FIG. 12 is a front and back photograph of necropsied tissue at Day 28 post-wounding for control and hypoxia primed viable placental tissue mixture (FPF) treated animals.

FIG. 13 is a photomicrograph of H&E stained wound tissue slides from control and hypoxia primed viable placental tissue mixture (FPF) treated animals.

FIG. 14 is a photomicrograph of Masson's trichrome stained wound tissue slides from control and hypoxia primed viable placental tissue mixture (FPF) treated animals.

FIG. 15 is a photomicrograph of Collagen IV stained wound tissue slides from control and hypoxia primed viable placental tissue mixture (FPF) treated animals.

FIG. 16A is a photomicrograph of aSMA stained wound tissue slides from control and hypoxia primed viable placental tissue mixture (FPF) treated animals.

FIG. 16B is a plot of blood vessel density of control and hypoxia primed viable placental tissue mixture (FPF) treated animals from aSMA stained wound tissue slides.

FIG. 16C is a plot of number of blood vessels vs vessel diameter size of control and hypoxia primed viable placental tissue mixture (FPF) treated animals from aSMA stained wound tissue slides.

FIG. 17A is a photomicrograph of CD31 stained wound tissue slides from control and hypoxia primed viable placental tissue mixture (FPF) treated animals.

FIG. 17B is a plot of blood vessel density of control and hypoxia primed viable placental tissue mixture (FPF) treated animals from CD31 stained wound tissue slides.

FIG. 17C is a plot of number of blood vessels vs vessel diameter size of control and hypoxia primed viable placental tissue mixture (FPF) treated animals from CD31 stained wound tissue slides.

FIG. 18 is a photomicrograph of CD31 and αSMA stained wound tissue slides from control and hypoxia primed viable placental tissue mixture (FPF) treated animals.

FIG. 19A is a photomicrograph of MPO stained wound tissue slides from control and hypoxia primed viable placental tissue mixture (FPF) treated animals.

FIG. 19B is a plot of % positive neutrophils of control and hypoxia primed viable placental tissue mixture (FPF) treated animals from MPO stained wound tissue slides.

FIG. 20A is a photomicrograph of CD163 stained wound tissue slides from control and hypoxia primed viable placental tissue mixture (FPF) treated animals.

FIG. 20B is a plot of % positive M2-macrophages of control and hypoxia primed viable placental tissue mixture (FPF) treated animals from CD163 stained wound tissue slides.

FIG. 21A is a plot of CINC-1 levels in wound tissue of control and hypoxia primed viable placental tissue mixture (FPF) treated animals.

FIG. 21B is a plot of CINC-2 levels in wound tissue of control and hypoxia primed viable placental tissue mixture (FPF) treated animals.

FIG. 21C is a plot of CINC-3 levels in wound tissue of control and hypoxia primed viable placental tissue mixture (FPF) treated animals.

FIG. 21D is a plot of LIX levels in wound tissue of control and hypoxia primed viable placental tissue mixture (FPF) treated animals.

FIG. 21E is a plot of IL-6 levels in wound tissue of control and hypoxia primed viable placental tissue mixture (FPF) treated animals.

FIG. 21F is a plot of L-Selectin levels in wound tissue of control and hypoxia primed viable placental tissue mixture (FPF) treated animals.

FIG. 21G is a plot of JAM-A levels in wound tissue of control and hypoxia primed viable placental tissue mixture (FPF) treated animals.

FIG. 21H is a plot of MIP-1a levels in wound tissue of control and hypoxia primed viable placental tissue mixture (FPF) treated animals.

FIG. 21I is a plot of RANTES levels in wound tissue of control and hypoxia primed viable placental tissue mixture (FPF) treated animals.

FIG. 21J is a plot of TREM-1 levels in wound tissue of control and hypoxia primed viable placental tissue mixture (FPF) treated animals.

FIG. 21K is a plot of Activin A levels in wound tissue of control and hypoxia primed viable placental tissue mixture (FPF) treated animals.

FIG. 21L is a plot of Fractalkine levels in wound tissue of control and hypoxia primed viable placental tissue mixture (FPF) treated animals.

FIG. 21M is a plot of Eotaxin levels in wound tissue of control and hypoxia primed viable placental tissue mixture (FPF) treated animals.

FIG. 21N is a plot of TWEAK R levels in wound tissue of control and hypoxia primed viable placental tissue mixture (FPF) treated animals.

FIG. 22 is a heatmap illustrating the fold change in genes expressed in control vs FPF-treated wounds.

FIG. 23 is a principal component analysis (PCA) plot identifying two principal components (PC1 and PC2) of control and hypoxia primed viable placental tissue mixture (FPF) treated animals.

FIG. 24 is a volcano plot of statistically significant differentially-expressed miRs of control and hypoxia primed viable placental tissue mixture (FPF) treated animals.

FIG. 25 is a timeline of the in vivo Rat Hind Limb Ischemic Model study. DM=Doppler measurement, IS=Ischemia surgery, Inj=hypoxia primed viable placental tissue mixture or Saline injection.

FIG. 26 is a graph showing hindlimb perfusion flux measurements for animals treated with hypoxia primed viable placental tissue mixture (FPF) and control. N=10. Reading are shown after animals had been under anesthesia under imager for 10 minutes. ns: not significant, **p<0.01, ***p<0.005.

FIG. 27 is a perfusion image for control and hypoxia primed viable placental tissue mixture (FPF) treated group after the animal was under anesthesia for 10 minutes at pre-surgery (D-1), the day of injection (one day post-ischemia induction) (DO), and Day 35 post-treatment (D35). Healthy limbs are on the left and ischemic/treated limbs are on the right.

FIG. 28A is a plot of CINC-1 levels in gracilis muscle of control and hypoxia primed viable placental tissue mixture (FPF) treated animals.

FIG. 28B is a plot of CINC-2 levels in gracilis muscle of control and hypoxia primed viable placental tissue mixture (FPF) treated animals.

FIG. 28C is a plot of RAGE levels in gracilis muscle of control and hypoxia primed viable placental tissue mixture (FPF) treated animals.

FIG. 28D is a plot of IL-13 levels in gracilis muscle of control and hypoxia primed viable placental tissue mixture (FPF) treated animals.

FIG. 28E is a plot of IL-22 levels in gracilis muscle of control and hypoxia primed viable placental tissue mixture (FPF) treated animals.

FIG. 28F is a plot of Galectin-3 levels in gracilis muscle of control and hypoxia primed viable placental tissue mixture (FPF) treated animals.

FIG. 28G is a plot of Erythropoietin levels in gracilis muscle of control and hypoxia primed viable placental tissue mixture (FPF) treated animals.

FIG. 28H is a plot of IL-7 levels in gracilis muscle of control and hypoxia primed viable placental tissue mixture (FPF) treated animals.

### DETAILED DESCRIPTION OF THE INVENTION

The present disclosure relates to methods of priming viable placental tissue; primed viable placental tissue and products thereof; bioactive materials formed in and isolated from the spent culture medium (conditioned medium) of the priming process and products thereof; and methods of use in regenerative medicine. Various priming techniques for priming the viable placental tissue are utilized in this disclosure including exposure to hypoxia, UV light, bioactive materials, or culture conditions, or combinations thereof. For example, viable placental tissue exposed to UV light can also be exposed to hypoxic conditions. Priming viable placental tissue results in an increase of cellular activity, antimicrobial activity, and an increased production of growth factors, peptides, antimicrobial peptides, cytokines, extracellular vesicles, exosomes, secretomes, microvesicles, extracellular matrix (ECM), and/or other bioactive materials over non-primed viable placental tissue. These enhanced therapeutic properties of the primed viable placental tissue over non-primed viable placental tissue allow for the primed viable placental tissue to provide a greater degree of therapeutic effectiveness in regenerative medicine than non-primed viable placental tissue. The primed viable placental tissue can be used in therapeutic applications as is or can be incorporated into primed viable placental product compositions comprising a pharmaceutically acceptable carrier.

During the priming process, the native viable placental cells, including mesenchymal stromal cells (MSCs), fibroblasts, and epithelial cells, have the benefit of being surrounded and embedded in their native placental tissue/ECM which is rich in a mixture of nutrients, proteins and signaling molecules. Because the MSCs are not isolated from their native placental tissue, the MSCs will maintain/preserve their stemness and other beneficial therapeutic properties during the priming process. Also, the other beneficial viable native cells including fibroblasts and epithelial cells will still be present in the placental tissue. Thus, there are many added benefits to priming viable placental tissue instead of just priming isolated/passaged cells.

Another benefit of the priming process is the increased production/secretion of bioactive materials produced during the priming process and found in the spent culture medium (conditioned medium). These bioactive materials, including but not limited to extracellular vesicles, exosomes, microvesicles, secretomes, cytokines, growth factors, peptides, antimicrobial peptides, or extracellular matrices, can be isolated from the spent culture medium and added to primed viable placental product compositions or made into therapeutic product compositions by themselves. Additionally, the spent culture medium (conditioned medium) can also be used as a therapeutic product composition.

The primed viable placental tissue and compositions disclosed herein can be used in regenerative medicine indications including, but not limited to wound management, sports medicine, orthopedics, trauma, ENT, dental, or tissue regeneration.

### I. Viable Placental Tissue

Viable placental tissue can include any or all tissue from the placenta and/or umbilical cord including, but not limited to the amnion, the chorion, the umbilical cord, Wharton's Jelly, and/or the whole placenta, and/or combinations thereof. Umbilical cord tissue comprises an amniotic epithelial tissue layer and a Wharton's Jelly tissue layer. The viable placental tissue can be the amniotic epithelial tissue layer of the umbilical cord which does not include the Wharton's Jelly layer. In some embodiments, the viable placental tissue is the amnion, the chorion, or the umbilical cord, or combinations thereof. In some embodiments, the viable placental tissue is the amnion. In other embodiments, the viable placental tissue is the chorion. In other embodiments, the viable placental tissue is the umbilical cord. In some embodiments, the umbilical cord is devoid of any blood vessel structures. In other embodiments, the viable placental tissue is the amniotic epithelial tissue layer of the umbilical cord (and does not include the Wharton's Jelly tissue layer). In other embodiments, the viable placental tissue is Wharton's Jelly tissue. In some embodiments, the viable placental tissue is the amnion, the chorion, the umbilical cord, or the amniotic epithelial layer of the umbilical cord, or combinations thereof. Viable placental tissue can include viable native cells including but not limited to MSCs, epithelial cells, and/or fibroblasts. Viable placental tissue also includes native ECM. In some embodiments, the viable placental tissue comprises one or more of MSCs, epithelial cells, or fibroblasts.

The placenta or umbilical cord can be harvested from a donor mammal after birth (natural or cesarean section) or at any time during the term of the pregnancy. The mammal preferably is a human (Homo sapiens), but can include other mammals such as other primates, murine, rabbit, cat, dog, pig, or equine. In some embodiments, the mammal is a human.

After collection from a donor, the placenta can be processed by aseptically cleaning it with Anticoagulant Citrate Dextrose Solution A, U.S.P. (ACD-A), phosphate buffer saline (PBS), or other suitable cleaning and rinsing solutions. Removal or partial depletion of unwanted parts from the placenta (e.g. vascularized tissue, blood, blood vessels, and/or trophoblast layer) can be done by methods known by one of skill in the art. The desired tissue, e.g. amnion, chorion, or umbilical cord, can be extracted from the placenta and separated from blood and other maternal components by methods known by one of skill in the art such as but not limited to blunt dissection. Unwanted cells including but not limited to cells such as one or more of viable immunogenic cells, trophoblasts, CD14+ macrophages, vascularized tissue-derived immunogenic cells, immunogenic maternal cells, maternal dendritic cells, or maternal leukocytes can be removed or partially depleted from the viable placental tissue by methods known by one of skill in the art. In some embodiments, the viable placental tissue is partially depleted of, substantially free of, or free of one or more of viable immunogenic cells, trophoblasts, CD14+ macrophages, vascularized tissue-derived immunogenic cells, immunogenic maternal cells, maternal dendritic cells, or maternal leukocytes. In some embodiments, the viable placental tissue comprises less than about 0.5%, or less than about 1%, or less than about 5% of CD14+ macrophages relative to the total number of native cells in the tissue. In some embodiments, the viable placental tissue is partially depleted of, substantially free of, or free of one or more of vascularized tissue, blood vessels, blood, or trophoblast layer. The viable placental tissue can be in various forms including, but not limited to sheets, wraps, grafts, pieces, minced pieces, crushed pieces, diced pieces, chopped pieces, cut pieces, chunks, or powder. The umbilical cord tissue can comprise one or more engineered channels. The viable placental tissue can be cryopreserved and then thawed for further processing or priming. The viable placental tissue can be lyophilized prior to further processing or priming. The lyophilized tissue can be rehydrated (reconstituted) with water or an aqueous solution such as saline solution prior to further processing or priming.

Prior to priming, the viable placental tissue can be further processed by incubating it in a culture medium which can contain additives, nutrients, and/or antibiotics. The culture medium can be a commercially available cell culture medium with or without additives as known by one of skill in the art. A non-limiting example of a culture medium includes Dulbecco's Modified Eagle's Medium (DMEM or DMEM low glucose) available from Sigma Aldrich. Non-limiting additives can be lysed platelets, antibiotics, fetal bovine serum (FBS), or human serum albumin. As used herein, lysed platelets mean human lysed platelets, also known as human platelet lysates. Lysed platelets provide a non-animal origin alternative to FBS. Lysed platelets are available commercially from STEMCELL™ Technologies or from Biological Industries, Inc. The culture medium can contain lysed platelets, FBS or human serum albumin at about 0.5%, or about 1%, or about 1.5%, or about 2%, or about 2.5%, or about 3%, or about 3.5%, or about 4%, or about 4.5%, or about 5%, or about 5.5%, or about 6%, or about 6.5%, or about 7%, or about 7.5%, or about 8%, or about 8.5%, or about 9%, or about 9.5%, or about 10%, or about 1% to about 10%, or about 1% to about 7%, or about 1% to about 5%, or about 1% to about 3%, or about 1% to about 2.5%. Another non-limiting example of a culture medium is commercially available chemically defined medium with or without serum. A non-limiting example of an antibiotic can be a penicillin-streptomycin solution such as Gibco™ pen-strep available from ThermoFisher Scientific. The incubation can take place in an incubator. The incubation conditions, e.g., the incubation time, the atmosphere conditions, and the temperature, can be established for the incubation. The incubator atmosphere can be humidified. The relative humidity (RH) level can be at from about 50% to about 99% RH, or from about 55% to about 99% RH, or from about 60% to about 99% RH, or from about 65% to about 99% RH, or from about 70% to about 99% RH, or from about 75% to about 99% RH, or from about 80% to about 99% RH, or from about 85% to about 99% RH, or from about 90% to about 99% RH, or from about 92% RH to about 99% RH, or from about 94% to about 99% RH, or from about 90% to about 98% RH, or from about 92% to about 98% RH, or from about 94% to about 98% RH, or from about 90% to about 96% RH, or from about 92% to about 96% RH, or from about 94% to about 96% RH, or about 90% RH, or about 91% RH, or about 92% RH, or about 93% RH, or about 94% RH, or about 95% RH, or about 96% RH, or about 97% RH, or about 98% RH, or about 99% RH, or greater than 50% RH, or greater than 55% RH, or greater than 60% RH, or greater than 65% RH, or greater than 70% RH, or greater than 75% RH, or greater than 80% RH, or greater than 85% RH, or greater than 90% RH. In some embodiments, the incubator is humidified at about 95% RH. The incubator atmosphere can contain CO₂. The CO₂ level can be from about 1% to about 10%, or from about 1% to about 7%, or from about 1% to about 5%, or from about 3% to about 7%, or from about 4% to about 6%, or about 5%. The incubation can take place at normoxic conditions. The incubator can be at a temperature during incubation of from about 20° C. to about 40° C., or from about 20° C. to about 30° C. or from about 25° C. to about 40° C., or from about 30° C. to about 40° C., or from about 35° C. to about 40° C., or from about 36° C. to about 38° C., or about 20° C., or about 25° C., or about 30° C., or about 35° C., or about 36° C., or about 37° C., or about 38° C., or about 39° C., or about 40° C. The incubation time can be about 1 to about 96 hours, or about 2 to about 96 hours, or about 3 to about 96 hours, or about 4 to about 96 hours, or about 5 to about 96 hours, or about 6 to about 96 hours, or about 12 to about 96 hours, or about 18 to about 96 hours, or about 24 to about 96 hours, or about 30 to about 96 hours, or about 36 to about 96 hours, or about 42 to about 96 hours, or about 48 to about 96 hours, or about 54 to about 96 hours, or about 60 to about 96 hours, or about 66 to about 96 hours, or about 72 to about 96 hours, or about 78 to about 96 hours, or about 84 to about 96 hours, or about 90 to about 96 hours, or about 1 to about 84 hours, or about 2 to about 84 hours, or about 3 to about 84 hours, or about 4 to about 84 hours, or about 5 to about 84 hours, or about 6 to about 84 hours, or about 12 to about 84 hours, or about 18 to about 84 hours, or about 24 to about 84 hours, or about 30 to about 84 hours, or about 36 to about 84 hours, or about 42 to about 84 hours, or about 48 to about 84 hours, or about 54 to about 84 hours, or about 60 to about 84 hours, or about 66 to about 84 hours, or about 72 to about 84 hours, or about 78 to about 84 hours, or about 1 to about 72 hours, or about 2 to about 72 hours, or about 3 to about 72 hours, or about 4 to about 72 hours, about 5 to about 72 hours, about 6 to about 72 hours, about 12 to about 72 hours, about 18 to about 72 hours, about 24 to about 72 hours, about 30 to about 72 hours, about 36 to about 72 hours, about 42 to about 72 hours, about 48 to about 72 hours, about 54 to about 72 hours, about 60 to about 72 hours, about 66 to about 72 hours, about 1 to about 60 hours, about 2 to about 60 hours, about 3 to about 60 hours, about 4 to about 60 hours, about 5 to about 60 hours, about 6 to about 60 hours, about 12 to about 60 hours, about 18 to about 60 hours, about 24 to about 60 hours, about 30 to about 60 hours, about 36 to about 60 hours, about 42 to about 60 hours, about 48 to about 60 hours, about 54 to about 60 hours, about 1 to about 54 hours, about 2 to about 54 hours, about 3 to about 54 hours, about 4 to about 54 hours, about 4 to about 54 hours, about 5 to about 54 hours, about 6 to about 54 hours, about 12 to about 54 hours, about 18 to about 54 hours, about 24 to about 54 hours, about 30 to about 54 hours, about 36 to about 54 hours, about 42 to about 54 hours, about 48 to about 54 hours, or about 1 to about 48 hours, or about 2 to about 48 hours, or about 3 to about 48 hours, or about 4 to about 48 hours, or about 5 to about 48 hours, or about 6 to about 48 hours, or about 12 to about 48 hours, or about 18 to about 48 hours, or about 24 to about 48 hours, or about 30 to about 48 hours, or about 36 to about 48 hours, or about 40 to about 48 hours, or about 1 to about 42 hours, or about 2 to about 42 hours, or about 3 to about 42 hours, or about 4 to about 42 hours, or about 5 to about 42 hours, or about 6 to about 42 hours, or about 12 to about 42 hours, or about 18 to about 42 hours, or about 24 to about 42 hours, or about 30 to about 42 hours, or about 36 to about 42 hours, or about 1 to about 36 hours, or about 2 to about 36 hours, or about 3 to about 36 hours, or about 4 to about 36 hours, or about 6 to about 36 hours, or about 12 to about 36 hours, or about 18 to about 36 hours, or about 24 to about 36 hours, or about 30 to about 36 hours, or about 1 to about 30 hours, or about 2 to about 30 hours, or about 3 to about 30 hours, or about 4 to about 30 hours, or about 5 to about 30 hours, or about 6 to about 30 hours, or about 12 to about 30 hours, or about 18 to about 30 hours, or about 24 to about 30 hours, or about 1 to about 24 hours, or about 2 to about 24 hours, or about 3 to about 24 hours, or about 4 to about 24 hours, or about 5 to about 24 hours, or about 6 to about 24 hours, or about 12 to about 24 hours, or about 18 to about 24 hours, or about 1 to about 18 hours, or about 2 to about 18 hours, or about 3 to about 18 hours, or about 4 to about 18 hours, or about 5 to about 18 hours, or about 6 to about 18 hours, or about 12 to about 18 hours, or about 1 to about 12 hours, or about 2 to about 12 hours, or about 3 to about 12 hours, or about 4 to about 12 hours, or about 5 to about 12 hours, or about 6 to about 12 hours, or about 1 to about 8 hours, or about 2 to about 8 hours, or about 3 to about 8 hours, or about 4 to about 8 hours, or about 4 to about 8 hours, or about 1 to about 6 hours, or about 2 to about 6 hours, or about 3 to about 6 hours, or about 4 to about 6 hours, or about 1 hour, or about 2 hours, or about 3 hours, or about 4 hours, or about 5 hours, or about 6 hours, or about 12 hours, or about 18 hours, or about 24 hours, or about 30 hours, or about 36 hours, or about 42 hours, or about 48 hours, or about 54 hours, or about 60 hours, or about 66 hours or about 72 hours, or about 78 hours, or about 84 hours, or about 90 hours, or about 96 hours. In some embodiments, the incubation time is about 4 hours to about 96 hours, or about 12 to about 36 hours, or about 24 hours. In some embodiments, the incubation takes place in an incubator. In some embodiments, the incubator atmosphere is humidified during incubation. In some embodiments, the incubator atmosphere includes about 5% CO₂ during incubation. In some embodiments, the temperature during incubation is about 37° C. In some embodiments, the culture medium comprises DMEM. In some embodiments, the culture medium comprises one or more of lysed platelets, antibiotics, fetal bovine serum (FBS), and/or human serum albumin. In some embodiments, the lysed platelets are included in the culture medium at about 1% to about 10%. In some embodiments, the FBS is included in the culture medium at about 1% to about 10%. In some embodiments, the human serum albumin is included in the culture medium at about 1% to about 10%. In some embodiments, the culture medium comprises DMEM plus about 5% FBS plus Gibco™ pen-strep. In some embodiments, the culture medium comprises a chemically defined medium with or without serum. In some embodiments, the antibiotic is a penicillin-streptomycin solution. Prior to priming, the processed viable placental tissue can be cryopreserved and then thawed for priming. Prior to priming, the processed viable placental tissue can be lyophilized. The lyophilized tissue can be rehydrated (reconstituted) with water or an aqueous solution such as saline solution prior to priming. Methods to remove and collect viable placental tissue (e.g., amnion, chorion, and umbilical cord tissue) from the placenta and process it are known by one of skill in the art examples of which are described and disclosed in US publication 2011/0212063, US publication 2011/0206776, and US publication 2017/0136071, all of which are incorporated herein by reference in their entirety.

The cell viability of the native cells in the viable placental tissue after processing, but before priming, can be at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 90%. The native cells can be at least one of MSCs, epithelial cells, or fibroblasts. The cell viability is the proportion of live cells as compared to the number of total cells at a given time. For example, in embodiments in which cell viability of the native cells in the viable placental tissue is at least about 70%, this means that at least about 70% of all the native cells in the viable placental tissue are viable cells. Determination of the cell viability can be done using methods known to one of skill in the art. For example, cells can be isolated from the viable placental tissue using enzymatic digestion and mixed with trypan blue for counting using an automated cell counter, e.g., Cellometer®, or hemocytometer. The viability of the cells can also be determined using flow cytometry or a live-dead assay kit such as the LIVE/DEAD® Viability/Cytotoxicity Kit or LIVE/DEAD® Cell Imaging Kit both available from ThermoFisher Scientific. In some embodiments, the viability of the native cells in the viable placental tissue after processing, but before priming is at least about 70%, wherein the native cells are at least one of MSCs, epithelial cells, or fibroblasts, and wherein the viable placental tissue is amnion tissue or chorion tissue. In some embodiments, the viability of the native cells in the viable placental tissue after processing, but before priming is at least about 40%, wherein the native cells are at least one of MSCs, epithelial cells, or fibroblasts, and wherein the viable placental tissue is umbilical cord tissue.

### II. Priming Techniques

### A. Hypoxia Priming

### 1. Hypoxia Priming Methods

Disclosed herein are methods for priming viable placental tissue by exposure of the tissue to hypoxic conditions. The methods comprise (a) providing the viable placental tissue, wherein the viable placental tissue comprises viable cells native to the placental tissue and (b) contacting the viable placental tissue with a culture medium at hypoxic conditions, thereby priming the viable placental tissue and generating spent culture medium. Optionally, (c) the primed viable placental tissue is separated from the spent culture medium. The hypoxia primed viable placental tissue can be separated from the spent (conditioned) medium or it can remain in contact with the spent (conditioned medium).

Prior to the hypoxia priming process, the viable placental tissue can be collected and processed as described herein. In some embodiments, the viable placental tissue is amnion tissue, chorion tissue, Wharton's Jelly tissue, or umbilical cord tissue, or mixtures thereof. In some embodiments, the viable cells comprise one or more of mesenchymal stromal cells (MSCs), epithelial cells, or fibroblasts. The viable placental tissue can be in many types of forms such as sheets, wraps, grafts, pieces, minced pieces, crushed pieces, diced pieces, chopped pieces, cut pieces, chunks, or powder. In some embodiments, the viable placental tissue is in the form of a sheet, wrap, or graft. In other embodiments, the viable placental tissue is in the form of minced pieces, or a powder. The viable placental tissue can be cryopreserved and then thawed prior to priming. The viable placental tissue can be lyophilized prior to priming. The lyophilized viable placental tissue can be rehydrated (reconstituted) with water or an aqueous solution such as saline solution prior to priming.

The hypoxia priming can take place in a hypoxia chamber or an incubator with the ability to control the atmospheric conditions inside the incubator at hypoxic conditions. The hypoxia chamber can be a self-contained and sealed chamber that fits inside existing laboratory incubators or can be a chamber/incubator with the ability to control the atmospheric conditions inside the chamber at hypoxic conditions. These hypoxia chambers and incubators are commercially available. The hypoxic conditions can include oxygen levels of from about 1% to about 10%, or from about 1% to about 9%, or from about 1% to about 8%, or from about 1% to about 7%, or from about 1% to about 6%, or from about 1% to about 5%, or from about 1% to about 4%, or from about 1% to about 3%, or from about 1% to about 2%, or about 10%, or about 9%, or about 8%, or about 7%, or about 6%, or about 5%, or about 4%, or about 3%, or about 2%, or about 1%, or less than about 10%, or less than about 9%, or less than about 8%, or less than about 7%, or less than about 6%, or less than about 5%, or less than about 4%, or less than about 3%, or less than about 2%, or less than about 1%. Normoxic conditions can include oxygen levels of from about 15% to about 21%, or from about 16% to about 21%, or from about 17% to about 21%, or from about 18% to about 21%, or from about 19% to about 21%, or from about 20% to about 21%, or about 21%, or about 20%, or about 19%, or about 18%, or about 17%, or about 16%, or about 15%. In some embodiments, the hypoxic conditions are at from about 1% to about 5%, or from about 1% to about 3%, or about 2% 02.

The hypoxia chamber/incubator atmosphere can be humidified during the priming process. The relative humidity (RH) level can be at levels as disclosed for Viable Placental Tissue supra. In some embodiments, the incubator is humidified at about 95% RH. The hypoxia chamber/incubator atmosphere can also contain CO₂ during the priming process. The CO₂ level can be from about 1% to about 10%, or from about 1% to about 7%, or from about 1% to about 5%, or from about 3% to about 7%, or from about 4% to about 6%, or about 5%. The hypoxia chamber/incubator can be at a temperature during the priming process of from about 20° C. to about 40° C., or from about 20° C. to about 30° C. or from about 25° C. to about 40° C., or from about 30° C. to about 40° C., or from about 35° C. to about 40° C., or from about 36° C. to about 38° C., or about 20° C., or about 25° C., or about 30° C., or about 35° C., or about 36° C., or about 37° C., or about 38° C., or about 39° C., or about 40° C. In some embodiments, the hypoxia chamber/incubator temperature is at about 37° C. during the priming process.

The exposure time for culturing under hypoxic conditions can be about 1 to about 96 hours, or about 2 to about 96 hours, or about 3 to about 96 hours, or about 4 to about 96 hours, or about 5 to about 96 hours, or about 6 to about 96 hours, or about 12 to about 96 hours, or about 18 to about 96 hours, or about 24 to about 96 hours, or about 30 to about 96 hours, or about 36 to about 96 hours, or about 42 to about 96 hours, or about 48 to about 96 hours, or about 54 to about 96 hours, or about 60 to about 96 hours, or about 66 to about 96 hours, or about 72 to about 96 hours, or about 78 to about 96 hours, or about 84 to about 96 hours, or about 90 to about 96 hours, or about 1 to about 84 hours, or about 2 to about 84 hours, or about 3 to about 84 hours, or about 4 to about 84 hours, or about 5 to about 84 hours, or about 6 to about 84 hours, or about 12 to about 84 hours, or about 18 to about 84 hours, or about 24 to about 84 hours, or about 30 to about 84 hours, or about 36 to about 84 hours, or about 42 to about 84 hours, or about 48 to about 84 hours, or about 54 to about 84 hours, or about 60 to about 84 hours, or about 66 to about 84 hours, or about 72 to about 84 hours, or about 78 to about 84 hours, or about 1 to about 72 hours, or about 2 to about 72 hours, or about 3 to about 72 hours, or about 4 to about 72 hours, about 5 to about 72 hours, about 6 to about 72 hours, about 12 to about 72 hours, about 18 to about 72 hours, about 24 to about 72 hours, about 30 to about 72 hours, about 36 to about 72 hours, about 42 to about 72 hours, about 48 to about 72 hours, about 54 to about 72 hours, about 60 to about 72 hours, about 66 to about 72 hours, about 1 to about 60 hours, about 2 to about 60 hours, about 3 to about 60 hours, about 4 to about 60 hours, about 5 to about 60 hours, about 6 to about 60 hours, about 12 to about 60 hours, about 18 to about 60 hours, about 24 to about 60 hours, about 30 to about 60 hours, about 36 to about 60 hours, about 42 to about 60 hours, about 48 to about 60 hours, about 54 to about 60 hours, about 1 to about 54 hours, about 2 to about 54 hours, about 3 to about 54 hours, about 4 to about 54 hours, about 4 to about 54 hours, about 5 to about 54 hours, about 6 to about 54 hours, about 12 to about 54 hours, about 18 to about 54 hours, about 24 to about 54 hours, about 30 to about 54 hours, about 36 to about 54 hours, about 42 to about 54 hours, about 48 to about 54 hours, or about 1 to about 48 hours, or about 2 to about 48 hours, or about 3 to about 48 hours, or about 4 to about 48 hours, or about 5 to about 48 hours, or about 6 to about 48 hours, or about 12 to about 48 hours, or about 18 to about 48 hours, or about 24 to about 48 hours, or about 30 to about 48 hours, or about 36 to about 48 hours, or about 40 to about 48 hours, or about 1 to about 42 hours, or about 2 to about 42 hours, or about 3 to about 42 hours, or about 4 to about 42 hours, or about 5 to about 42 hours, or about 6 to about 42 hours, or about 12 to about 42 hours, or about 18 to about 42 hours, or about 24 to about 42 hours, or about 30 to about 42 hours, or about 36 to about 42 hours, or about 1 to about 36 hours, or about 2 to about 36 hours, or about 3 to about 36 hours, or about 4 to about 36 hours, or about 6 to about 36 hours, or about 12 to about 36 hours, or about 18 to about 36 hours, or about 24 to about 36 hours, or about 30 to about 36 hours, or about 1 to about 30 hours, or about 2 to about 30 hours, or about 3 to about 30 hours, or about 4 to about 30 hours, or about 5 to about 30 hours, or about 6 to about 30 hours, or about 12 to about 30 hours, or about 18 to about 30 hours, or about 24 to about 30 hours, or about 1 to about 24 hours, or about 2 to about 24 hours, or about 3 to about 24 hours, or about 4 to about 24 hours, or about 5 to about 24 hours, or about 6 to about 24 hours, or about 12 to about 24 hours, or about 18 to about 24 hours, or about 1 to about 18 hours, or about 2 to about 18 hours, or about 3 to about 18 hours, or about 4 to about 18 hours, or about 5 to about 18 hours, or about 6 to about 18 hours, or about 12 to about 18 hours, or about 1 to about 12 hours, or about 2 to about 12 hours, or about 3 to about 12 hours, or about 4 to about 12 hours, or about 5 to about 12 hours, or about 6 to about 12 hours, or about 1 to about 8 hours, or about 2 to about 8 hours, or about 3 to about 8 hours, or about 4 to about 8 hours, or about 4 to about 8 hours, or about 1 to about 6 hours, or about 2 to about 6 hours, or about 3 to about 6 hours, or about 4 to about 6 hours, or about 1 hour, or about 2 hours, or about 3 hours, or about 4 hours, or about 5 hours, or about 6 hours, or about 12 hours, or about 18 hours, or about 24 hours, or about 30 hours, or about 36 hours, or about 42 hours, or about 48 hours, or about 54 hours, or about 60 hours, or about 66 hours or about 72 hours, or about 78 hours, or about 84 hours, or about 90 hours, or about 96 hours.

The culture medium used for priming the tissue under hypoxic conditions can contain additives, nutrients, and/or antibiotics. The culture medium can be a commercially available cell culture medium with or without additives as known by one of skill in the art. A non-limiting example of a culture medium includes Dulbecco's Modified Eagle's Medium (DMEM) available from Sigma Aldrich. Non-limiting additives can be lysed platelets, antibiotics, fetal bovine serum (FBS), or human serum albumin. For example, the culture medium can be DMEM containing about 1% to about 10% lysed platelets, about 1% to about 10% FBS, or about 1% to about 10% human serum albumin. The culture medium can contain lysed platelets, FBS or human serum albumin at about 0.5%, or about 1%, or about 1.5%, or about 2%, or about 2.5%, or about 3%, or about 3.5%, or about 4%, or about 4.5%, or about 5%, or about 5.5%, or about 6%, or about 6.5%, or about 7%, or about 7.5%, or about 8%, or about 8.5%, or about 9%, or about 9.5%, or about 10%, or about 1% to about 10%, or about 1% to about 7%, or about 1% to about 5%, or about 1% to about 3%, or about 1% to about 2.5%. Another non-limiting example of a culture medium is commercially available chemically defined medium with or without serum. A non-limiting example of an antibiotic can be a penicillin-streptomycin solution such as Gibco™ pen-strep available from ThermoFisher Scientific. In some embodiments, the culture medium comprises DMEM. In some embodiments, the culture medium comprises nutrients. In some embodiments, the culture medium comprises serum. In some embodiments, the culture medium comprises one or more of lysed platelets, antibiotics, fetal bovine serum (FBS), or human serum albumin. In some embodiments, the lysed platelets are included in the culture medium at about 1% to about 10%. In some embodiments, the FBS is included in the culture medium at about 1% to about 10%. In some embodiments, the human serum albumin is included in the culture medium at about 1% to about 10%. In some embodiments, the culture medium comprises a chemically defined medium with or without serum. In some embodiments, the culture medium comprises DMEM containing about 2.5% FBS.

Bioactive materials can be added to the culture medium used for priming. The addition of bioactive materials to the culture medium can contribute to the priming effects on the viable placental tissue. A bioactive material is a substance or compound having a biological effect upon a living organism, tissue, or cell. Examples of bioactive materials include, but are not limited to growth factors, peptides, antimicrobial peptides, cytokines, enzymes, extracellular vesicles, extracellular matrices, TNF-alpha, and interferon-gamma. In some embodiments, the one or more bioactive materials comprise TNF-alpha at an amount of about 5 to about 50 ng/ml, interferon-gamma at an amount of about 5 to about 50 ng/ml, or a combination of TNF-alpha at an amount of about 5 to about 50 ng/ml and interferon-gamma at an amount of about 5 to about 50 ng/ml. The bioactive materials can be in the form of nanoparticles. In some embodiments, the method further comprises adding an effective amount of one or more bioactive materials to the culture medium to be used in the priming process.

The resulting hypoxia primed viable placental tissues can be amnion tissue, chorion tissue, or umbilical cord tissue, or mixtures thereof, and can be in many types of forms such as sheets, wraps, grafts, pieces, minced pieces, crushed pieces, diced pieces, chopped pieces, cut pieces, chunks, or powder. In some embodiments, the resulting hypoxia primed viable placental tissue is in the form of a sheet, wrap, or graft. In other embodiments, the resulting hypoxia primed viable placental tissue is in the form of minced pieces, or a powder. The viable cells in the resulting hypoxia primed viable placental tissue can include but are not limited to one or more of mesenchymal stromal cells (MSCs), epithelial cells, or fibroblasts.

Indicators that the viable placental tissue that has been primed by hypoxic conditions include the increased secretion of angiogenic factors such as vascular endothelial growth factor A (VEGF-A) and/or cytokines such as the interleukin-1 receptor antagonist (IL-1RA) in the primed viable placental tissue as compared to non-primed viable placental tissue cultured under normoxia conditions. As compared to a non-primed viable placental tissue cultured under normoxia conditions, the hypoxia primed viable placental tissue can exhibit enhanced VEGF-A function as determined by a cellular functional assay of about a 1 fold to about a 10 fold increase in activity, or about a 1 fold to about a 9 fold increase in activity, or about a 1 fold to about a 8 fold increase in activity, or about a 1 fold to about a 7 fold increase in activity, or about a 1 fold to about a 6 fold increase in activity, or about a 1 fold to about a 5 fold increase in activity, or about a 1 fold to about a 4 fold increase in activity, or about a 1 fold to about a 3 fold increase in activity, or about a 1 fold to about a 2 fold increase in activity, or about a 2 fold to about a 10 fold increase in activity, or about a 2 fold to about a 9 fold increase in activity, or about a 2 fold to about a 8 fold increase in activity, or about a 2 fold to about a 7 fold increase in activity, or about a 2 fold to about a 6 fold increase in activity, or about a 2 fold to about a 5 fold increase in activity, or about a 2 fold to about a 4 fold increase in activity, or about a 2 fold to about a 3 fold increase in activity, or about a 3 fold to about a 10 fold increase in activity, or about a 3 fold to about a 9 fold increase in activity, or about a 3 fold to about a 8 fold increase in activity, or about a 3 fold to about a 7 fold increase in activity, or about a 3 fold to about a 6 fold increase in activity, or about a 3 fold to about a 5 fold increase in activity, or about a 3 fold to about a 4 fold increase in activity, or about a 4 fold to about a 10 fold increase in activity, or about a 4 to about a 9 fold increase in activity, or about a 4 to about a 8 fold increase in activity, or about a 4 to about a 7 fold increase in activity, or about a 4 to about a 6 fold increase in activity, or about a 4 to about a 5 fold increase in activity, or about a 5 fold to about a 10 fold increase in activity, or about a 5 fold to about a 9 fold increase in activity, or about a 5 fold to about a 8 fold increase in activity, or about a 5 fold to about a 7 fold increase in activity, or about a 5 fold to about a 6 fold increase in activity, or at least a 1 fold increase in activity, or at least a 2 fold increase in activity, or at least a 3 fold increase in activity, or at least a 4 fold increase in activity, or at least a 5 fold increase in activity, or at least a 6 fold increase in activity, or at least a 7 fold increase in activity, or at least a 8 fold increase in activity, or at least a 9 fold increase in activity, or at least a 10 fold increase in activity. As compared to a non-primed viable placental tissue cultured under normoxia conditions, the hypoxia primed viable placental tissue can exhibit increased VEGF-A secretion of about a 1 fold to about a 10 fold increase, or about a 1 fold to about a 9 fold increase, or about a 1 fold to about a 8 fold increase, or about a 1 fold to about a 7 fold increase, or about a 1 fold to about a 6 fold increase, or about a 1 fold to about a 5 fold increase, or about a 1 fold to about a 4 fold increase, or about a 1 fold to about a 3 fold increase, or about a 1 fold to about a 2 fold increase, or about a 2 fold to about a 10 fold increase, or about a 2 fold to about a 9 fold increase, or about a 2 fold to about a 8 fold increase, or about a 2 fold to about a 7 fold increase, or about a 2 fold to about a 6 fold increase, or about a 2 fold to about a 5 fold increase, or about a 2 fold to about a 4 fold increase, or about a 2 fold to about a 3 fold increase, or about a 3 fold to about a 10 fold increase, or about a 3 fold to about a 9 fold increase, or about a 3 fold to about a 8 fold increase, or about a 3 fold to about a 7 fold increase, or about a 3 fold to about a 6 fold increase, or about a 3 fold to about a 5 fold increase, or about a 3 fold to about a 4 fold increase, or at least a 1 fold increase, or at least a 2 fold increase, or at least a 3 fold increase, or at least a 4 fold increase, or at least a 5 fold increase, or at least a 6 fold increase, or at least a 7 fold increase, or at least a 8 fold increase, or at least a 9 fold increase, or at least a 10 fold increase. As compared to a non-primed viable placental tissue cultured under normoxia conditions, the hypoxia primed viable placental tissue can exhibit increased IL-1RA secretion of about a 1 fold to about a 10 fold increase, or about a 1 fold to about a 9 fold increase, or about a 1 fold to about a 8 fold increase, or about a 1 fold to about a 7 fold increase, or about a 1 fold to about a 6 fold increase, or about a 1 fold to about a 5 fold increase, or about a 1 fold to about a 4 fold increase, or about a 1 fold to about a 3 fold increase, or about a 1 fold to about a 2 fold increase, or about a 2 fold to about a 10 fold increase, or about a 2 fold to about a 9 fold increase, or about a 2 fold to about a 8 fold increase, or about a 2 fold to about a 7 fold increase, or about a 2 fold to about a 6 fold increase, or about a 2 fold to about a 5 fold increase, or about a 2 fold to about a 4 fold increase, or about a 2 fold to about a 3 fold increase, or about a 3 fold to about a 10 fold increase, or about a 3 fold to about a 9 fold increase, or about a 3 fold to about a 8 fold increase, or about a 3 fold to about a 7 fold increase, or about a 3 fold to about a 6 fold increase, or about a 3 fold to about a 5 fold increase, or about a 3 fold to about a 4 fold increase, or at least a 1 fold increase, or at least a 2 fold increase, or at least a 3 fold increase, or at least a 4 fold increase, or at least a 5 fold increase, or at least a 6 fold increase, or at least a 7 fold increase, or at least a 8 fold increase, or at least a 9 fold increase, or at least a 10 fold increase. In some embodiments, the hypoxia primed viable placental tissue primed under hypoxia conditions exhibits enhanced angiogenic factor (VEGF-A) function (preferably about a 3 fold to about a 5 fold increase in activity) as determined by a cellular functional assay, an increase in VEGF-A secretion (preferably about a 1 fold to about a 3 fold increase), and/or an increase in IL-1RA secretion (preferably about a 1 fold to about a 3 fold increase), as compared to non-primed viable placental tissue cultured under normoxia conditions.

Interleukin-1 (IL-1) is identified as a key pro-inflammatory cytokine that mediates the body's fight against extraneous foreign bodies. IL-1 is shown to exert strong proinflammatory activities in ischemic wound settings, which left uncontrolled can work to prevent wound healing. IL-1 includes IL-1 alpha (IL-1A) and IL-1 beta (IL1B), and their effects are supervised by several naturally occurring inhibitors. The interleukin-1 receptor antagonist (IL-1RA) is the natural antagonist of IL-1 and is an anti-inflammatory factor. Thus, IL-1RA can lower inflammation in a wound setting, allowing for the healing process to begin. IL-1 signaling also plays a key role in other inflammatory diseases. In vitro studies of osteoarthritis show that continuous joint inflammation results in cartilage degradation, and that the inhibition of IL-1 by IL-1RA can be used to combat this disease. Inflammatory diseases have also been linked to bone re-absorption and osteoclast activity. IL-1B specifically is shown to be a strong stimulator of bone reabsorption using in vivo and in vitro models. IL-1RA can thus be valued as a therapy to lower the inflammatory processes to limit osteoclastogenesis and lower the rate of bone reabsorption in the osteoporosis disease model. Therefore, viable placental tissue primed using hypoxia which contain increased secretion levels of IL-1RA is a therapeutic solution in regenerative medicine for treatment of a variety regenerative diseases and conditions such as inflammatory diseases.

Vascular endothelial growth factor (VEGF) is the principal angiogenic growth factor that mediates the prominent cellular responses involved in angiogenesis. VEGF-A is considered to be the dominant inducer to the growth of blood vessels. VEGF improved skeletal muscle repair through modulation of angiogenesis, regeneration, and fibrosis in the impaired muscle. Bone is a highly vascularized organ and angiogenesis plays an important role in osteogenesis and delivering VEGF has been shown to be effective in skeletal and bone repair and regeneration. VEGF also plays an important role in regeneration of hypostome and tentacles in hydra. The most prevalent cause of ischemia, an inadequate blood supply for tissue demand, is progressive atherosclerotic stenosis of cardiac or limb arteries, which can be compounded by microvascular dysfunction in metabolic syndromes such as diabetes. The increased generation of new vascular networks through delivery of specific growth factors, such as VEGF-A, is an attractive strategy to restore perfusion to ischemic tissues and fill this unmet clinical need. Therefore, viable placental tissue primed using hypoxia which contain increased secretion levels of VEGF-A and enhanced angiogenic factor (VEGF-A) function is a therapeutic solution in regenerative medicine for treatment of a variety of regenerative diseases and conditions.

The methods can further comprise collecting the spent culture medium of step (c). The methods can further comprise isolating one or more bioactive materials from the spent culture medium. The isolated one or more bioactive materials can include, but is not limited to extracellular vesicles, exosomes, microvesicles, secretomes, cytokines, growth factors, peptides, antimicrobial peptides, or extracellular matrices.

During the priming process, it was observed that the original dead or dying cells in the placental tissue are sloughed off, whereas the viable cells look even more robust after priming. The cell viability of the native cells in the viable placental tissue after hypoxia priming can be at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 90%. The native cells can be at least one of MSCs, epithelial cells, or fibroblasts. The cell viability is the proportion of live cells as compared to the number of total cells at a given time. Determination of the cell viability can be done using methods known to one of skill in the art. In some embodiments, the cell viability of the native cells in the viable placental tissue after hypoxia priming is at least about 70%.

The hypoxia primed viable placental tissue can further be cryopreserved or lyophilized after the priming process and can be stored in suitable packaging. The cryopreserved hypoxia primed viable placental tissue can be thawed prior to use. The lyophilized hypoxia primed viable placental tissue can be rehydrated (reconstituted) with water or an aqueous solution such as saline solution prior to use or used as is.

### 2. Compositions of Hypoxia Primed Viable Placental Tissue

Also disclosed herein are compositions comprising hypoxia primed viable placental tissues. In some embodiments the compositions comprise the hypoxia primed viable placental tissues produced by any of the hypoxia priming methods described herein. In some embodiments, the hypoxia primed viable placental tissue was primed by exposure to hypoxic conditions at the any of the hypoxic conditions described herein. In some embodiments, the hypoxic conditions were from about 1% to about 5% 02 or from about 1% to about 2% 02, or about 2% 02. The hypoxia primed viable placental tissue can be amnion tissue, chorion tissue, Wharton's Jelly tissue, or umbilical cord tissue, or mixtures thereof. In some embodiments, the viable cells of the hypoxia primed viable placental tissue comprise one or more of mesenchymal stromal cells (MSCs), epithelial cells, or fibroblasts. In some embodiments, the hypoxia primed viable placental tissue exhibits enhanced angiogenic factor (VEGF-A) function (preferably about a 3 fold to about a 5 fold increase in activity) as determined by a cellular functional assay, an increase in VEGF-A secretion (preferably about a 1 fold to about a 3 fold increase), and/or an increase in IL-1RA secretion (preferably about a 1 fold to about a 3 fold increase) as compared to non-primed viable placental tissue cultured under normoxia conditions. The hypoxia primed viable placental tissue can be in many types of forms such as sheets, wraps, grafts, pieces, minced pieces, crushed pieces, diced pieces, chopped pieces, cut pieces, chunks, or powder. In some embodiments, the hypoxia primed viable placental tissue is in the form of a sheet, wrap, or graft. In other embodiments, the hypoxia primed viable placental tissue is in the form of minced pieces, or a powder.

The compositions of hypoxia primed viable placental tissue can further comprise a pharmaceutically acceptable carrier. In some embodiments, the pharmaceutically acceptable carrier is aqueous based. In other embodiments, the pharmaceutically acceptable carrier is non-aqueous based. In still other embodiments, the pharmaceutically acceptable carrier is a powder. Examples of suitable pharmaceutically acceptable carriers include but are not limited to suspensions, solutions, gels, pastes, emulsions, creams, lotions, ointments, or powders. The pharmaceutically acceptable carriers can comprise excipients that will not cause significant damage to the viability of the cells. These excipients include but are not limited to saline solutions, water, buffers, buffer solutions, sugars, trehalose, proteins, starches, emulsifiers, gelling agents, preservatives, antimicrobials, pH adjusters, and surfactants. The carriers can also comprise active pharmaceutical ingredients. The carriers can be formulated for various routes of administration including but not limited to topical, injection, or surgical, and can include coatings for medical devices or implants.

The compositions of hypoxia primed viable placental tissue can further comprise one or more bioactive materials including but not limited to extracellular vesicles, exosomes, microvesicles, secretomes, cytokines, growth factors, peptides, antimicrobial peptides, extracellular matrices, TNF-alpha (preferably at an amount from about 5 to about 50 ng/ml), interferon-gamma (preferably at an amount from about 5 to about 50 ng/ml), or nanoparticles thereof. In some embodiments, the bioactive materials comprise one or more of the bioactive materials produced by any of the hypoxia priming methods of viable placental tissue as described herein and isolated from the spent (conditioned) culture medium. The compositions of the hypoxia primed viable placental tissue can be cryopreserved or lyophilized.

In some embodiments, the compositions of hypoxia primed viable placental tissue are partially depleted of, substantially free of, or free of one or more of viable immunogenic cells, trophoblasts, CD14+ macrophages, vascularized tissue-derived immunogenic cells, immunogenic maternal cells, maternal dendritic cells, or maternal leukocytes. In some embodiments, the compositions of hypoxia primed viable placental tissue comprise less than about 0.5%, or less than about 1%, or less than about 5% of CD14+ macrophages relative to the total number of native cells in the tissue. In some embodiments, the compositions of hypoxia primed viable placental tissue are partially depleted of, substantially free of, or free of one or more of vascularized tissue, blood vessels, blood, or trophoblast layer.

The cell viability of the native cells in the hypoxia primed viable placental tissue can be at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 90%. The native cells can be at least one of MSCs, epithelial cells, or fibroblasts. The cell viability is the proportion of live cells as compared to the number of total cells at a given time. Determination of the cell viability can be done using methods known to one of skill in the art. In some embodiments, the cell viability of the native cells in the hypoxia primed viable placental tissue is at least about 70%.

### 3. Compositions of Byproducts of Hypoxia Primed Viable Placental Tissue

The spent culture medium resulting from the hypoxia priming process, which has become a conditioned medium and is a byproduct of the hypoxia priming process, can be collected, and used for various purposes. During the hypoxia priming process, bioactive materials can be produced by the viable placental tissue. Some of these bioactive materials, which are also byproducts of the hypoxia priming process, can remain in the hypoxia primed viable placental tissue and some of these bioactive materials can be deposited into the culture medium. If any bioactive materials were added to the culture medium used for the hypoxia priming, some of the bioactive materials that are produced by the viable placental tissue can the same bioactive materials as those that were added, and some can be different bioactive materials from those that were added. Examples of these bioactive materials that are formed during the hypoxia priming process include, but are not limited to extracellular vesicles, exosomes, microvesicles, secretomes, cytokines, growth factors, peptides, antimicrobial peptides, and extracellular matrices. In some embodiments, the spent culture medium itself containing one or more bioactive materials can be used in regenerative medicine compositions. In other embodiments, one or more bioactive materials can be isolated from the spent culture medium by methods known to one of skill in the art. The byproducts of the hypoxia priming process, i.e., the spent (conditioned) culture medium and/or the bioactive materials therein, can be used in regenerative medicine compositions. Also disclosed herein are compositions of the spent (conditioned) medium resulting from the hypoxia priming of viable placental tissue and compositions of one or more bioactive materials isolated from the spent (conditioned) medium. The compositions can further comprise a pharmaceutically acceptable carrier. In some embodiments, the pharmaceutically acceptable carrier is aqueous based. In other embodiments, the pharmaceutically acceptable carrier is non-aqueous based. In still other embodiments, the pharmaceutically acceptable carrier is a powder. Examples of suitable pharmaceutically acceptable carriers include but are not limited to suspensions, solutions, gels, pastes, emulsions, creams, lotions, ointments, or powders. The pharmaceutically acceptable carriers can comprise excipients. These excipients include but are not limited to saline solutions, water, buffers, buffer solutions, sugars, trehalose, proteins, starches, emulsifiers, gelling agents, preservatives, antimicrobials, pH adjusters, and surfactants. The carriers can also comprise active pharmaceutical ingredients. The carriers can be formulated for various routes of administration including but not limited to topical, injection, or surgical, and can include coatings for medical devices or implants. The compositions can be cryopreserved or lyophilized.

### 4. Methods of Using Hypoxia Primed Viable Placental Tissue Compositions and Byproduct Compositions

Also disclosed herein are methods of using hypoxia primed viable placental tissue and compositions thereof in regenerative medicine. In vivo animal studies as exemplified herein have shown that hypoxia primed viable placental tissue mixture accelerates ischemic wound healing and closure with improved tissue architecture, is conducive for a pro-angiogenic and an anti-inflammatory wound microenvironment, induces transcriptional regulation favorable for ischemic wound healing, favorably modulates miRs that help in redirecting tissue healing conducive for appropriate restoration of the dermal tissue, and increases tissue perfusion upon ischemic tissue injury. Disclosed are methods of regenerating, replacing, or repairing diseased, damaged, or injured body tissue of a subject, the methods comprising administering to the subject any one of the hypoxia primed viable placental tissue compositions disclosed herein.

Also disclosed herein are methods of using in regenerative medicine the compositions of the spent (conditioned) medium from the hypoxia priming of viable placental tissue and/or compositions of the byproducts of the hypoxia priming process found in and isolated from the spent (conditioned) medium. Disclosed are methods of regenerating, replacing, or repairing diseased, damaged, or injured body tissue of a subject, the methods comprising administering to the subject any one of the compositions of the spent (conditioned) medium from the hypoxia priming of viable placental tissue and/or compositions of the byproducts of the hypoxia priming process found in and isolated from the spent (conditioned) medium disclosed herein.

Examples of body tissue that can be diseased, damaged, or injured includes, but is not limited to tendons, cartilage, ligaments, periosteum, perichondrium, synovium, fascia, mesentery, sinew, dental tissue, gums, fistulas, nasal septum, vaginal wall tissue, abdominal wall tissue, peritoneum, tumor resection sites, dermal tissue, dermal wounds, dermal lesions, dermal abrasions, dermal burns, first degree burns, partial thickness burns, full thickness burns, epidermal wounds, congenital wounds, toxic epidermal necrolysis, epidermolysis bullosa, pyoderma gangrenosum, dermal ulcers, diabetic ulcers, diabetic foot ulcers, venous ulcers, venous let ulcers, pressure ulcers, arterial ulcers, decubitus ulcers, stasis ulcers, ischemic ulcers, chronic wounds, acute wounds, surgical wounds, internal wounds, hernia, bladder tissue, keloids, adhesions, organ lacerations, organ defects, diseased organ tissue, epithelial defects, spinal cord tissue, nerve tissue, tympanic membranes, and/or mucous membranes.

The compositions can be administered topically, subcutaneously, surgically, or by injection, e.g., intramuscular injection. The compositions can be administered by coating the composition onto the surface of a medical device implant and implanting the coated device into the subject.

### B. UV Light Priming

### 1. UV Light Priming Methods

Disclosed herein are methods for priming viable placental tissue by exposure of the tissue to ultraviolet (UV) light. The methods comprise (a) providing the viable placental tissue, wherein the viable placental tissue comprises viable cells native to the viable placental tissue, and (b) exposing the viable placental tissue to UV light, thereby priming the viable placental tissue. The methods can optionally further comprise (c) contacting the UV light primed viable placental tissue with a culture medium and thereby generating spent culture medium, and optionally, (d) separating the UV light primed viable placental tissue from the spent culture medium. In some embodiments, the UV light primed viable placental tissue is cultured in a culture medium. The UV light primed viable placental tissue after culturing can be separated from the spent (conditioned) medium or it can remain in contact with the spent (conditioned medium). In some embodiments, the UV light primed viable placental tissue is not cultured.

Prior to the UV light priming process, the viable placental tissue can be collected and processed as described herein. In some embodiments, the viable placental tissue is amnion tissue, chorion tissue, Wharton's Jelly tissue, or umbilical cord tissue, or mixtures thereof. In some embodiments, the viable cells comprise one or more of mesenchymal stromal cells (MSCs), epithelial cells, or fibroblasts. The viable placental tissue can be in many types of forms such as sheets, wraps, grafts, pieces, minced pieces, crushed pieces, diced pieces, chopped pieces, cut pieces, chunks, or powder. In some embodiments, the viable placental tissue is in the form of a sheet, wrap, or graft. In other embodiments, the viable placental tissue is in the form of minced pieces, or a powder. The viable placental tissue can be cryopreserved and then thawed prior to priming. The viable placental tissue can be lyophilized prior to priming. The lyophilized viable placental tissue can be rehydrated (reconstituted) with water or an aqueous solution such as saline solution prior to priming.

The UV light region covers the wavelength range of 100-400 nm and is divided into three bands: UVA (315-400 nm), UVB (280-315 nm) and UVC (100-280 nm). The UV light can be UVA, UVB, or UVC or combinations thereof. In some embodiments, the UV light is UVB light. The source of the UV light can be from any suitable UV light source, such as a UV lamp. A suitable UV lamp is commercially available from ThermoFisher Scientific Cat #: 95034. The UV light as described herein is artificial and does not include ambient room lighting. The UV light source can emit UV light at a wavelength at any single wavelength within any of the UV wavelength band ranges of UVA, UVB, or UVC. In some embodiments, the UV light source emits a wavelength of about 302 nm. In some embodiments, the UV light source emits a wavelength of about 312 nm. In other embodiments, the UV light source emits a wavelength of about 252 nm. In still other embodiments, the UV light source emits a wavelength of about 365 nm. The wattage output of the UV light can be from about 4 to about 40 watts, or from about 4 to about 20 watts, or from about 4 to about 15 watts, or from about 4 to about 8 watts, or about 4 to about 6 watts, or from about 6 to about 40 watts, or from about 6 to about 20 watts, or from about 6 to about 15 watts, or from about 6 to about 8 watts, or from about 8 to about 40 watts, or from about 8 to about 20 watts, or from about 8 to about 15 watts, or about 4 watts, or about 6 watts, or about 8 watts, or about 15 watts, or about 20 watts, or about 40 watts. In some embodiments, the wattage output of the UV light is about 8 watts.

The viable placental tissue sample can be positioned at a suitable distance away from the UV light source so the sample can be adequately exposed with UV light. For example, if the sample is a flat sheet or piece with a top and bottom surface, then one side of the sample can be exposed to UV light, or both sides of the sample can be exposed to UV light by exposing one side first and then flipping the sample over to expose the opposite side. In some embodiments, the distance is from about 1 cm to about 100 cm, or from about 1 cm to about 90 cm, or from about 1 cm to about 80 cm, or from about 1 cm to about 70 cm, or from about 1 cm to about 60 cm, or from about 1 cm to about 50 cm, or from about 1 cm to about 40 cm, or from about 1 cm to about 30 cm, or from about 1 cm to about 20 cm, or from about 1 cm to about 15 cm, or from about 1 cm to about 10 cm, or from about 5 cm to about 100 cm, or from about 5 cm to about 90 cm, or from about 5 cm to about 80 cm, or from about 5 cm to about 70 cm, or from about 5 cm to about 60 cm, or from about 5 cm to about 50 cm, or from about 5 cm to about 40 cm, or from about 5 cm to about 30 cm, or from about 5 cm to about 20 cm, or from about 5 cm to about 15 cm, or from about 5 cm to about 10 cm, or from about 10 cm to about 100 cm, or from about 10 cm to about 90 cm, or from about 10 cm to about 80 cm, or from about 10 cm to about 70 cm, or from about 10 cm to about 60 cm, or from about 10 cm to about 50 cm, or from about 10 cm to about 40 cm, or from about 10 cm to about 30 cm, or from about 10 cm to about 20 cm, or from about 10 to about 15 cm, or about 1 cm, or about 2 cm, or about 3 cm, or about 4 cm, or about 5 cm, or about 6 cm, or about 7 cm, or about 8 cm, or about 9 cm, or about 10 cm, or about 11 cm, or about 12 cm, or about 13 cm, or about 14 cm, or about 15 cm, or about 16 cm, or about 17 cm, or about 18 cm, or about 19 cm, or about 20 cm, or about 30 cm, or about 40 cm, or about 50 cm, or about 60 cm, or about 70 cm, or about 80 cm, or about 90 cm, or about 100 cm.

The exposure time to the UV light is temporary and can be from about 5 seconds to about 10 min, or from about 5 seconds to about 9 min, or from about 5 seconds to about 8 min, or from about 5 seconds to about 7 min, or from about 5 seconds to about 6 min, or from about 5 seconds to about 5 min, or from about 5 seconds to about 4½ minutes, or from about 5 seconds to about 4 minutes, or from about 5 seconds to about 3½ minutes, or from about 5 seconds to about 180 seconds, or from about 5 seconds to about 150 seconds, or from about 5 seconds to about 120 seconds, or from about 5 seconds to about 90 seconds, or from about 5 seconds to about 60 seconds, or from about 5 seconds to about 50 seconds, or from about 5 seconds to about 40 seconds, or from about 5 seconds to about 30 seconds, or from about 5 seconds to about 20 seconds, or from about 10 seconds to about 10 min, or from about 10 seconds to about 9 min, or from about 10 seconds to about 8 min, or from about 10 seconds to about 7 min, or from about 10 seconds to about 6 min, or from about 10 seconds to about 5 min, or from about 10 seconds to about 4½ minutes, or from about 10 seconds to about 4 minutes, or from about 10 seconds to about 3½ minutes, or from about 10 seconds to about 180 seconds, or from about 10 seconds to about 150 seconds, or from about 10 seconds to about 120 seconds, or from about 10 seconds to about 90 seconds, or from about 10 seconds to about 60 seconds, or from about 10 seconds to about 50 seconds, or from about 10 seconds to about 40 seconds, or from about 10 seconds to about 30 seconds, or from about 10 seconds to about 20 seconds, or from about 15 seconds to about 10 min, or from about 15 seconds to about 9 min, or from about 15 seconds to about 8 min, or from about 15 seconds to about 7 min, or from about 15 seconds to about 6 min, or from about 15 seconds to about 5 min, or from about 15 seconds to about 4½ minutes, or from about 15 seconds to about 4 minutes, or from about 15 seconds to about 3½ minutes, or from about 15 seconds to about 180 seconds, or from about 15 seconds to about 150 seconds, or from about 15 seconds to about 120 seconds, or from about 15 seconds to about 90 seconds, or from about 15 seconds to about 60 seconds, or from about 15 seconds to about 50 seconds, or from about 15 seconds to about 40 seconds, or from about 15 seconds to about 30 seconds, or from about 15 seconds to about 20 seconds, from about 20 seconds to about 10 min, or from about 20 seconds to about 9 min, or from about 20 seconds to about 8 min, or from about 20 seconds to about 7 min, or from about 20 seconds to about 6 min, or from about 20 seconds to about 5 min, or from about 20 seconds to about 4½ minutes, or from about 20 seconds to about 4 minutes, or from about 20 seconds to about 3½ minutes, or from about 20 seconds to about 180 seconds, or from about 20 seconds to about 150 seconds, or from about 20 seconds to about 120 seconds, or from about 20 seconds to about 90 seconds, or from about 20 seconds to about 60 seconds, or from about 20 seconds to about 50 seconds, or from about 5 seconds to about 40 seconds, or from about 20 seconds to about 30 seconds, or about 5 seconds, or about 10 seconds, or about 15 seconds, or about 20 seconds, or about 25 seconds, or about 30 seconds, or about 35 seconds, or about 40 seconds, or about 45 seconds, or about 50 seconds, or about 55 seconds or about 60 seconds, or about 70 seconds, or about 80 seconds, or about 90 seconds, or about 100 seconds, or about 110 seconds, or about 120 seconds, or about 130 seconds, or about 140 seconds, or about 150 seconds, or about 160 seconds, or about 170 seconds, or about 180 seconds, or about 3½ minutes, or about 4 minutes, or about 4½ minutes, or about 5 minutes, or about 6 minutes, or about 7 minutes, or about 8 minutes, or about 9 minutes, or about 10 minutes. In some embodiments, the exposure time is from about 10 seconds to about 4 minutes, or from about 20 seconds to about 180 seconds.

The culturing step (c) can take place in an incubator at normoxia conditions. The incubator atmosphere can be humidified during the culturing process. The relative humidity (RH) level can be at levels as disclosed for Viable Placental Tissue supra. The incubator atmosphere can also contain CO₂ during the culturing process. The CO₂ level can be from about 1% to about 10%, or from about 1% to about 7%, or from about 1% to about 5%, or from about 3% to about 7%, or from about 4% to about 6%, or about 5%. The incubator can be at a temperature during the culturing process of from about 20° C. to about 40° C., or from about 20° C. to about 30° C. or from about 25° C. to about 40° C., or from about 30° C. to about 40° C., or from about 35° C. to about 40° C., or from about 36° C. to about 38° C., or about 20° C., or about 25° C., or about 30° C., or about 35° C., or about 36° C., or about 37° C., or about 38° C., or about 39° C., or about 40° C. In some embodiments, the incubator temperature is at about 37° C. during the culturing process.

The exposure time during the culturing step (c) can be at time ranges as disclosed for Hypoxia Priming supra.

The culture medium used in the culturing step (c) can contain additives, nutrients, and/or antibiotics. The culture medium can be a commercially available cell culture medium with or without additives as known by one of skill in the art. A non-limiting example of a culture medium includes Dulbecco's Modified Eagle's Medium (DMEM) available from Sigma Aldrich. Non-limiting additives can be lysed platelets, antibiotics, fetal bovine serum (FBS), or human serum albumin. For example, the culture medium can be DMEM containing about 1% to about 10% lysed platelets, about 1% to about 10% FBS, or about 1% to about 10% human serum albumin. The culture medium can contain lysed platelets, FBS or human serum albumin at about 0.5%, or about 1%, or about 1.5%, or about 2%, or about 2.5%, or about 3%, or about 3.5%, or about 4%, or about 4.5%, or about 5%, or about 5.5%, or about 6%, or about 6.5%, or about 7%, or about 7.5%, or about 8%, or about 8.5%, or about 9%, or about 9.5%, or about 10%, or about 1% to about 10%, or about 1% to about 7%, or about 1% to about 5%, or about 1% to about 3%, or about 1% to about 2.5%. Another non-limiting example of a culture medium is commercially available chemically defined medium with or without serum. A non-limiting example of an antibiotic can be a penicillin-streptomycin solution such as Gibco™ pen-strep available from ThermoFisher Scientific. In some embodiments, the culture medium comprises DMEM. In some embodiments, the culture medium comprises nutrients. In some embodiments, the culture medium comprises serum. In some embodiments, the culture medium comprises one or more of lysed platelets, antibiotics, fetal bovine serum (FBS), or human serum albumin. In some embodiments, the lysed platelets are included in the culture medium at about 1% to about 10%. In some embodiments, the FBS is included in the culture medium at about 1% to about 10%. In some embodiments, the human serum albumin is included in the culture medium at about 1% to about 10%. In some embodiments, the culture medium comprises a chemically defined medium with or without serum. In some embodiments, the culture medium comprises DMEM containing about 2.5% FBS.

Bioactive materials can be added to the culture medium used for the culturing step (c). The addition of bioactive materials to the culture medium can contribute to the priming effects on the viable placental tissue. A bioactive material is a substance or compound having a biological effect upon a living organism, tissue, or cell. Examples of bioactive materials include, but are not limited to growth factors, peptides, antimicrobial peptides, cytokines, enzymes, extracellular vesicles, extracellular matrices, TNF-alpha, and interferon-gamma. In some embodiments, the one or more bioactive materials comprise TNF-alpha at an amount of about 5 to about 50 ng/ml, interferon-gamma at an amount of about 5 to about 50 ng/ml, or a combination of TNF-alpha at an amount of about 5 to about 50 ng/ml and interferon-gamma at an amount of about 5 to about 50 ng/ml. The bioactive materials can be in the form of nanoparticles. In some embodiments, the method further comprises adding an effective amount of one or more bioactive materials to the culture medium to be used in the culturing step (c).

The resulting UV light primed viable placental tissues can be amnion tissue, chorion tissue, or umbilical cord tissue, or mixtures thereof, and can be in many types of forms such as sheets, wraps, grafts, pieces, minced pieces, crushed pieces, diced pieces, chopped pieces, cut pieces, chunks, or powder. In some embodiments, the resulting UV light primed viable placental tissue is in the form of a sheet, wrap, or graft. In other embodiments, the resulting UV light primed viable placental tissue is in the form of minced pieces, or a powder. The viable cells in the resulting UV light primed viable placental tissue can include but are not limited to one or more of mesenchymal stromal cells (MSCs), epithelial cells, or fibroblasts.

Indicators that the viable placental tissue that has been primed by UV light include the increased secretion of antimicrobial peptides such as human beta-defensin-2 (HBD-2). HBD-2 is a cysteine-rich cationic low molecular weight antimicrobial peptide that exhibits antimicrobial activity against gram-negative bacteria and candida. Therefore, priming by UV light can be a method to enhance the antimicrobial property of the viable placental tissue in regenerative medicine uses. As compared to a non-primed viable placental tissue, the UV primed viable placental tissue can exhibit increased HBD-2 secretion of about a 1 fold to about a 10 fold increase, or about a 1 fold to about a 9 fold increase, or about a 1 fold to about a 8 fold increase, or about a 1 fold to about a 7 fold increase, or about a 1 fold to about a 6 fold increase, or about a 1 fold to about a 5 fold increase, or about a 1 fold to about a 4 fold increase, or about a 1 fold to about a 3 fold increase, or about a 1 fold to about a 2 fold increase, or about a 2 fold to about a 10 fold increase, or about a 2 fold to about a 9 fold increase, or about a 2 fold to about a 8 fold increase, or about a 2 fold to about a 7 fold increase, or about a 2 fold to about a 6 fold increase, or about a 2 fold to about a 5 fold increase, or about a 2 fold to about a 4 fold increase, or about a 2 fold to about a 3 fold increase, or about a 3 fold to about a 10 fold increase, or about a 3 fold to about a 9 fold increase, or about a 3 fold to about a 8 fold increase, or about a 3 fold to about a 7 fold increase, or about a 3 fold to about a 6 fold increase, or about a 3 fold to about a 5 fold increase, or about a 3 fold to about a 4 fold increase, or about a 4 fold to about a 10 fold increase, or about a 4 fold to about a 9 fold increase, or about a 4 fold to about a 8 fold increase, or about a 4 fold to about a 7 fold increase, or about a 4 fold to about a 6 fold increase, or about a 4 fold to about a 5 fold increase, or at least a 1 fold increase, or at least a 2 fold increase, or at least a 3 fold increase, or at least a 4 fold increase, or at least a 5 fold increase, or at least a 6 fold increase, or at least a 7 fold increase, or at least a 8 fold increase, or at least a 9 fold increase, or at least a 10 fold increase. In some embodiments, the UV light primed viable placental tissue exhibits an increased HBD-2 secretion (preferably about a 1 fold to about a 4 fold increase) as compared to viable placental tissue not exposed to UV light (non-primed).

The methods can further comprise collecting the spent culture medium of step (d) and isolating from the spent culture medium one or more bioactive materials formed in the culture medium during step (b) and (c). The isolated one or more bioactive materials can include, but is not limited to extracellular vesicles, exosomes, microvesicles, secretomes, cytokines, growth factors, peptides, antimicrobial peptides, or extracellular matrices.

The cell viability of the native cells in the viable placental tissue after UV light priming can be at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 90%. The native cells can be at least one of MSCs, epithelial cells, or fibroblasts. The cell viability is the proportion of live cells as compared to the number of total cells at a given time. Determination of the cell viability can be done using methods known to one of skill in the art. In some embodiments, the cell viability of the native cells in the viable placental tissue after UV light priming is at least about 70%.

The UV light primed viable placental tissue can further be cryopreserved or lyophilized after the priming process or after the priming-culturing process and can be stored in suitable packaging. The cryopreserved UV light primed viable placental tissue can be thawed prior to use. The lyophilized UV light primed viable placental tissue can be rehydrated (reconstituted) with water or an aqueous solution such as saline solution prior to use or used as is.

### 2. Compositions of UV Light Primed Viable Placental Tissue

Also disclosed herein are compositions comprising UV light primed viable placental tissues. In some embodiments the compositions comprise UV light primed viable placental tissues produced by any of the UV light priming methods described herein. In some embodiments, the UV light primed viable placental tissue was primed by exposure of the tissues to UV light. In some embodiments, the UV light was UVA light, or UVB light, or combinations thereof. In some embodiments, the UV light was UVB light. The UV light primed viable placental tissue can be amnion tissue, chorion tissue, Wharton's Jelly tissue, or umbilical cord tissue, or mixtures thereof. In some embodiments, the viable cells of the UV light primed viable placental tissue comprise one or more of mesenchymal stromal cells (MSCs), epithelial cells, or fibroblasts. In some embodiments, the UV light primed viable placental tissue exhibits an increase in HBD-2 secretion (preferably about a 1 fold to about a 4 fold increase) as compared to non-primed viable placental tissue. The UV light primed viable placental tissue can be in many types of forms such as sheets, wraps, grafts, pieces, minced pieces, crushed pieces, diced pieces, chopped pieces, cut pieces, chunks, or powder. In some embodiments, the UV light primed viable placental tissue is in the form of a sheet, wrap, or graft. In other embodiments, the UV light primed viable placental tissue is in the form of minced pieces, or a powder.

The compositions of UV light primed viable placental tissue can further comprise a pharmaceutically acceptable carrier. In some embodiments, the pharmaceutically acceptable carrier is aqueous based. In other embodiments, the pharmaceutically acceptable carrier is non-aqueous based. In still other embodiments, the pharmaceutically acceptable carrier is a powder. Examples of suitable pharmaceutically acceptable carriers include but are not limited to suspensions, solutions, gels, pastes, emulsions, creams, lotions, ointments, or powders. The pharmaceutically acceptable carriers can comprise excipients that will not cause significant damage to the viability of the cells. These excipients include but are not limited to saline solutions, water, buffers, buffer solutions, sugars, trehalose, proteins, starches, emulsifiers, gelling agents, preservatives, antimicrobials, pH adjusters, and surfactants. The carriers can also comprise active pharmaceutical ingredients. The carriers can be formulated for various routes of administration including but not limited to topical, injection, or surgical, and can include coatings for medical devices or implants.

The compositions of UV light primed viable placental tissue can further comprise one or more bioactive materials including but not limited to extracellular vesicles, exosomes, microvesicles, secretomes, cytokines, growth factors, peptides, antimicrobial peptides, extracellular matrices, TNF-alpha (preferably at an amount from about 5 to about 50 ng/ml), interferon-gamma (preferably at an amount from about 5 to about 50 ng/ml), or nanoparticles thereof. In some embodiments, the bioactive materials comprise one or more of the bioactive materials produced by any of the UV light priming methods of viable placental tissue wherein the UV light primed tissue was cultured as described herein and isolated from the spent (conditioned) culture medium. The compositions of the UV light primed viable placental tissue can be cryopreserved or lyophilized.

In some embodiments, the compositions of UV light primed viable placental tissue are partially depleted of, substantially free of, or free of one or more of viable immunogenic cells, trophoblasts, CD14+ macrophages, vascularized tissue-derived immunogenic cells, immunogenic maternal cells, maternal dendritic cells, or maternal leukocytes. In some embodiments, the compositions of UV light primed viable placental tissue comprise less than about 0.5%, or less than about 1%, or less than about 5% of CD14+ macrophages relative to the total number of native cells in the tissue. In some embodiments, the compositions of UV light primed viable placental tissue are partially depleted of, substantially free of, or free of one or more of vascularized tissue, blood vessels, blood, or trophoblast layer.

The cell viability of the native cells in the UV light primed viable placental tissue can be at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 90%. The native cells can be at least one of MSCs, epithelial cells, or fibroblasts. The cell viability is the proportion of live cells as compared to the number of total cells at a given time. Determination of the cell viability can be done using methods known to one of skill in the art. In some embodiments, the cell viability of the native cells in the UV light primed viable placental tissue is at least about 70%.

### 3. Compositions of Byproducts of UV Light Primed Viable Placental Tissue

The spent culture medium resulting from the culturing process when conducted after the UV light priming process can be collected and used for various purposes. During the UV light priming process, bioactive materials can be produced by the viable placental tissue. When the UV light primed viable placental tissue is cultured in a culture medium after the priming process, some of these bioactive materials, which are byproducts of the UV light priming process, can remain in the UV light primed viable placental tissue and some of these bioactive materials can be deposited into the culture medium. Thus, the spent culture medium can be considered a conditioned medium and a byproduct of the UV priming process. If any bioactive materials were added to the culture medium used for the culturing of the UV light primed viable placental tissue, some of the bioactive materials that are produced by the viable placental tissue during the UV light priming process can the same bioactive materials as those that were added, and some can be different bioactive materials from those that were added. Examples of these bioactive materials that are formed during the UV light priming process include, but are not limited to extracellular vesicles, exosomes, microvesicles, secretomes, cytokines, growth factors, peptides, antimicrobial peptides, and extracellular matrices. In some embodiments, the spent culture medium itself containing one or more bioactive materials can be used in regenerative medicine compositions. In other embodiments, one or more bioactive materials can be isolated from the spent culture medium by methods known to one of skill in the art. The byproducts of the UV light priming process, i.e., the spent (conditioned) culture medium and/or the bioactive materials therein, can be used in regenerative medicine compositions. Also disclosed herein are compositions of the spent (conditioned) medium resulting from the culturing of UV light primed viable placental tissue and compositions of one or more bioactive materials isolated from the spent (conditioned) medium. The compositions can further comprise a pharmaceutically acceptable carrier. In some embodiments, the pharmaceutically acceptable carrier is aqueous based. In other embodiments, the pharmaceutically acceptable carrier is non-aqueous based. In still other embodiments, the pharmaceutically acceptable carrier is a powder. Examples of suitable pharmaceutically acceptable carriers include but are not limited to suspensions, solutions, gels, pastes, emulsions, creams, lotions, ointments, or powders. The pharmaceutically acceptable carriers can comprise excipients. These excipients include but are not limited to saline solutions, water, buffers, buffer solutions, sugars, trehalose, proteins, starches, emulsifiers, gelling agents, preservatives, antimicrobials, pH adjusters, and surfactants. The carriers can also comprise active pharmaceutical ingredients. The carriers can be formulated for various routes of administration including but not limited to topical, injection, or surgical, and can include coatings for medical devices or implants. The compositions can be cryopreserved or lyophilized.

### 4. Methods of Using UV Light Primed Viable Placental Tissue Compositions and Byproduct Compositions

Also disclosed herein are methods of using UV light primed viable placental tissue and compositions thereof in regenerative medicine. Disclosed are methods of regenerating, replacing, or repairing diseased, damaged, or injured body tissue of a subject, the methods comprising administering to the subject any one of the UV light primed viable placental tissue compositions disclosed herein.

Also disclosed herein are methods of using in regenerative medicine the compositions of the spent (conditioned) medium from the UV light priming-culturing of viable placental tissue and/or compositions of the byproducts of the UV light priming-culturing process found in and isolated from the spent (conditioned) medium. Disclosed are methods of regenerating, replacing, or repairing diseased, damaged, or injured body tissue of a subject, the methods comprising administering to the subject any one of the compositions of the spent (conditioned) medium from the UV light priming-culturing of viable placental tissue and/or compositions of the byproducts of the UV light priming-culturing process found in and isolated from the spent (conditioned) medium disclosed herein.

Examples of body tissue that can be diseased, damaged, or injured includes, but is not limited to tendons, cartilage, ligaments, periosteum, perichondrium, synovium, fascia, mesentery, sinew, dental tissue, gums, fistulas, nasal septum, vaginal wall tissue, abdominal wall tissue, peritoneum, tumor resection sites, dermal tissue, dermal wounds, dermal lesions, dermal abrasions, dermal burns, first degree burns, partial thickness burns, full thickness burns, epidermal wounds, congenital wounds, toxic epidermal necrolysis, epidermolysis bullosa, pyoderma gangrenosum, dermal ulcers, diabetic ulcers, diabetic foot ulcers, venous ulcers, venous let ulcers, pressure ulcers, arterial ulcers, decubitus ulcers, stasis ulcers, ischemic ulcers, chronic wounds, acute wounds, surgical wounds, internal wounds, hernia, bladder tissue, keloids, adhesions, organ lacerations, organ defects, diseased organ tissue, epithelial defects, spinal cord tissue, nerve tissue, tympanic membranes, and/or mucous membranes.

The compositions can be administered topically, subcutaneously, surgically, or by injection, e.g., intramuscular injection. The compositions can be administered by coating the composition onto the surface of a medical device implant and implanting the coated device into the subject.

### C. Bioactive Material Priming

### 1. Bioactive Material Priming Methods

Disclosed herein are methods for priming viable placental tissue by exposure of the tissue to bioactive materials. A bioactive material is a substance or compound having a biological effect upon a living organism, tissue, or cell. Examples of bioactive materials include, but are not limited to growth factors, peptides, antimicrobial peptides, cytokines, enzymes, extracellular vesicles, extracellular matrices, TNF-alpha, and interferon-gamma. The bioactive materials can be in the form of nanoparticles. The methods comprise (a) providing the viable placental tissue, wherein the viable placental tissue comprises viable cells native to the viable placental tissue and (b) contacting the tissue with a culture medium comprising an effective amount of one or more bioactive materials, thereby priming the viable placental tissue and generating spent culture medium. Optionally, (c) the bioactive material primed viable placental tissue is separated from the spent culture medium. In some embodiments, the one or more bioactive materials are growth factors, peptides, antimicrobial peptides, cytokines, enzymes, extracellular vesicles, extracellular matrices, tumor necrosis factor alpha (TNF-alpha), interferon-gamma, or nanoparticles thereof. In some embodiments, the one or more bioactive materials comprise TNF-alpha, interferon-gamma, or a combination of TNF-alpha and interferon-gamma. The bioactive material TNF-alpha is a prototypic cytokine of the TNF superfamily and is involved in the regulation of biological process including cell proliferation, differentiation, apoptosis, lipid metabolism, and coagulation. The bioactive material interferon-gamma (INF-γ), which is the only type II INF cytokine, has been shown to play a critical role in inducing and modulating an array of immune responses including both innate and adaptive immune responses. The amount of TNG-alpha can be from about 5 to 50 ng/ml, or from about 5 to 45 ng/ml, or from about 5 to about 40 ng/ml, or from about 5 to about 35 ng/ml, or from about 5 to about 30 ng/ml, or from about 5 to 25 ng/ml, or from about 5 to 20 ng/ml, or from about 5 to 15 ng/ml, or from about 5 to 10 ng/ml, or from about 10 to about 50 ng/ml, or from about 10 to about 45 ng/ml, or from about 10 to about 40 ng/ml, or from about 10 to about 35 ng/ml, or from about 10 to about 30 ng/ml, or from about 10 to about 25 ng/ml, or from about 10 to about 20 ng/ml, or from about 10 to about 15 ng/ml, or about 5 ng/ml, or about 10 ng/ml, or about 15 ng/ml, or about 20 ng/ml, or about 25 ng/ml, or about 30 ng/ml, or about 35 ng/ml, or about 40 ng/ml, or about 45 ng/ml, or about 50 ng/ml. In some embodiments, the one or more bioactive materials comprise TNF-alpha at an amount of from about 5 to about 50 ng/ml, interferon-gamma at an amount of from about 5 to about 50 ng/ml, or a combination of TNF-alpha at an amount of from about 5 to about 50 ng/ml and interferon-gamma at an amount of from about 5 to about 50 ng/ml. In some embodiments, the one or more bioactive materials comprise TNF-alpha at an amount of about 10 ng/ml, interferon-gamma at an amount of about 10 ng/ml, or a combination of TNF-alpha at an amount of about 10 ng/ml and interferon-gamma at an amount of about 10 ng/ml. The bioactive material primed viable placental tissue can be separated from the spent (conditioned) medium or it can remain in contact with the spent (conditioned medium).

Prior to the bioactive material priming process, the viable placental tissue can be collected and processed as described herein. In some embodiments, the viable placental tissue is amnion tissue, chorion tissue, Wharton's Jelly tissue, or umbilical cord tissue, or mixtures thereof. In some embodiments, the viable cells comprise one or more of mesenchymal stromal cells (MSCs), epithelial cells, or fibroblasts. The viable placental tissue can be in many types of forms such as sheets, wraps, grafts, pieces, minced pieces, crushed pieces, diced pieces, chopped pieces, cut pieces, chunks, or powder. In some embodiments, the viable placental tissue is in the form of a sheet, wrap, or graft. In other embodiments, the viable placental tissue is in the form of minced pieces, or a powder. The viable placental tissue can be cryopreserved and then thawed prior to bioactive material priming. The viable placental tissue can be lyophilized prior to bioactive material priming. The lyophilized viable placental tissue can be rehydrated (reconstituted) with water or an aqueous solution such as saline solution prior to bioactive material priming.

The culture medium used for the bioactive material priming can be a commercially available cell culture medium with or without additives as known by one of skill in the art. A non-limiting example of a culture medium includes Dulbecco's Modified Eagle's Medium (DMEM or DMEM low glucose) available from Sigma Aldrich. Non-limiting additives can be lysed platelets, antibiotics, fetal bovine serum (FBS), or human serum albumin. The culture medium can contain lysed platelets, FBS or human serum albumin at about 0.5%, or about 1%, or about 1.5%, or about 2%, or about 2.5%, or about 3%, or about 3.5%, or about 4%, or about 4.5%, or about 5%, or about 5.5%, or about 6%, or about 6.5%, or about 7%, or about 7.5%, or about 8%, or about 8.5%, or about 9%, or about 9.5%, or about 10%, or about 1% to about 10%, or about 1% to about 7%, or about 1% to about 5%, or about 1% to about 3%, or about 1% to about 2.5%. Another non-limiting example of a culture medium is commercially available chemically defined medium with or without serum. A non-limiting example of an antibiotic can be a penicillin-streptomycin solution such as Gibco™ pen-strep available from ThermoFisher Scientific. In some embodiments, the culture medium comprises DMEM. In some embodiments, the culture medium comprises nutrients. In some embodiments, the culture medium comprises serum. In some embodiments, the culture medium comprises one or more of lysed platelets, antibiotics, FBS, and/or human serum albumin. In some embodiments, the lysed platelets are included in the culture medium at about 1% to about 10%. In some embodiments, the FBS is included in the culture medium at about 1% to about 10%. In some embodiments, the human serum albumin is included in the culture medium at about 1% to about 10%. In some embodiments, the culture medium comprises DMEM plus about 5% FBS plus Gibco™ pen-strep. In some embodiments, the culture medium comprises a chemically defined medium with or without serum. In some embodiments, the antibiotic is a penicillin-streptomycin solution

The bioactive material priming can take place in an incubator. The incubation conditions, e.g., the incubation time, the atmosphere conditions, and the temperature, can be established for the priming. In some embodiments, the bioactive material priming takes place in an incubator.

The incubator atmosphere can be humidified during bioactive material priming. The relative humidity (RH) level can be at levels as disclosed for Viable Placental Tissue supra. In some embodiments, the incubator is humidified at about 95% RH. In some embodiments, the incubator atmosphere is humidified during bioactive material priming. In some embodiments, the incubator is humidified at about 95% RH.

The incubator atmosphere can contain CO₂ during bioactive material priming. The CO₂ level can be from about 1% to about 10%, or from about 1% to about 7%, or from about 1% to about 5%, or from about 3% to about 7%, or from about 4% to about 6%, or about 5%. The incubator can be at normoxia conditions during bioactive material priming. In some embodiments, the incubator atmosphere includes about 5% CO₂ during bioactive material priming. In some embodiments, the bioactive material priming takes place at normoxic conditions.

The incubator can be at a temperature during bioactive material priming of from about 20° C. to about 40° C., or from about 20° C. to about 30° C. or from about 25° C. to about 40° C., or from about 30° C. to about 40° C., or from about 35° C. to about 40° C., or from about 36° C. to about 38° C., or about 20° C., or about 25° C., or about 30° C., or about 35° C., or about 36° C., or about 37° C., or about 38° C., or about 39° C., or about 40° C. In some embodiments, the temperature during bioactive material priming is about 37° C.

The bioactive material priming time, i.e., incubation time, can be at incubation time ranges as disclosed for Viable Placental Tissue supra.

The resulting bioactive material primed viable placental tissues can be amnion tissue, chorion tissue, or umbilical cord tissue, or mixtures thereof, and can be in many types of forms such as sheets, wraps, grafts, pieces, minced pieces, crushed pieces, diced pieces, chopped pieces, cut pieces, chunks, or powder. In some embodiments, the resulting bioactive material primed viable placental tissue is in the form of a sheet, wrap, or graft. In other embodiments, the resulting bioactive material primed viable placental tissue is in the form of minced pieces, or a powder. The viable cells in the resulting bioactive material primed viable placental tissue can include but are not limited to one or more of mesenchymal stromal cells (MSCs), epithelial cells, or fibroblasts.

The bioactive material primed viable placental tissue can exhibit one or more of the following increased therapeutic regenerative properties including but not limited to angiogenesis, anti-inflammatory, chemoattractant, antimicrobial, antioxidant or antifibrosis as compared to non-primed viable placental tissue as determined in vitro such as with ELISA and/or Multi-plex analysis, and/or in vivo.

The methods can further comprise collecting the spent culture medium of step (c). the methods can further comprise isolating one or more bioactive materials from the spent culture medium. The isolated one or more bioactive materials can include, but is not limited to extracellular vesicles, exosomes, microvesicles, secretomes, cytokines, growth factors, peptides, antimicrobial peptides, or extracellular matrices.

The cell viability of the native cells in the viable placental tissue after bioactive material priming can be at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 90%. The native cells can be at least one of MSCs, epithelial cells, or fibroblasts. The cell viability is the proportion of live cells as compared to the number of total cells at a given time. Determination of the cell viability can be done using methods known to one of skill in the art. In some embodiments, the cell viability of the native cells in the viable placental tissue after bioactive material priming is at least about 70%.

The bioactive material primed viable placental tissue can further be cryopreserved or lyophilized after the priming process and can be stored in suitable packaging. The cryopreserved bioactive material primed viable placental tissue can be thawed prior to use. The lyophilized bioactive material primed viable placental tissue can be rehydrated (reconstituted) with water or an aqueous solution such as saline solution prior to use or used as is. The bioactive priming methods of viable placental tissue can be combined with other viable placental tissue priming methods such as hypoxia priming, UV light priming, or UV light plus hypoxia priming as described herein.

### 2. Compositions of Bioactive Material Priming

Also disclosed herein are compositions comprising bioactive material primed viable placental tissues. In some embodiments the compositions comprise the bioactive material primed viable placental tissues produced by any of the bioactive material priming methods described herein. In some embodiments, the bioactive material primed viable placental tissue was primed by exposure to one or more bioactive materials. In some embodiments, the bioactive materials comprise growth factors, peptides, cytokines, enzymes, extracellular vesicles, extracellular matrices, TNF-alpha, interferon-gamma, or nanoparticles thereof. In some embodiments, the one or more bioactive materials comprise TNF-alpha, interferon-gamma, or a combination of TNF-alpha and interferon-gamma. The bioactive material primed viable placental tissue can be amnion tissue, chorion tissue, Wharton's Jelly tissue, or umbilical cord tissue, or mixtures thereof. In some embodiments, the viable cells of the bioactive material primed viable placental tissue comprise one or more of mesenchymal stromal cells (MSCs), epithelial cells, or fibroblasts. In some embodiments, the bioactive material primed viable placental tissue exhibits one or more of the following increased therapeutic regenerative properties comprising angiogenesis, anti-inflammatory, chemoattractant, antimicrobial, antioxidant or antifibrosis as compared to non-primed viable placental tissue as determined in vitro such as with ELISA and/or Multi-plex analysis, and/or in vivo. The bioactive material primed viable placental tissue can be in many types of forms such as sheets, wraps, grafts, pieces, minced pieces, crushed pieces, diced pieces, chopped pieces, cut pieces, chunks, or powder. In some embodiments, the bioactive material primed viable placental tissue is in the form of a sheet, wrap, or graft. In other embodiments, the bioactive material primed viable placental tissue is in the form of minced pieces, or a powder.

The compositions of bioactive material primed viable placental tissue can further comprise a pharmaceutically acceptable carrier. In some embodiments, the pharmaceutically acceptable carrier is aqueous based. In other embodiments, the pharmaceutically acceptable carrier is non-aqueous based. In still other embodiments, the pharmaceutically acceptable carrier is a powder. Examples of suitable pharmaceutically acceptable carriers include but are not limited to suspensions, solutions, gels, pastes, emulsions, creams, lotions, ointments, or powders. The pharmaceutically acceptable carriers can comprise excipients that will not cause significant damage to the viability of the cells. These excipients include but are not limited to saline solutions, water, buffers, buffer solutions, sugars, trehalose, proteins, starches, emulsifiers, gelling agents, preservatives, antimicrobials, pH adjusters, and surfactants. The carriers can also comprise active pharmaceutical ingredients. The carriers can be formulated for various routes of administration including but not limited to topical, injection, or surgical, and can include coatings for medical devices or implants.

The compositions of bioactive material primed viable placental tissue can further comprise one or more bioactive materials including but not limited to extracellular vesicles, exosomes, microvesicles, secretomes, cytokines, growth factors, peptides, antimicrobial peptides, extracellular matrices, TNF-alpha (preferably at an amount from about 5 to about 50 ng/ml), interferon-gamma (preferably at an amount from about 5 to about 50 ng/ml), or nanoparticles thereof. In some embodiments, the bioactive materials comprise one or more of the bioactive materials produced by any of the bioactive material priming methods of viable placental tissue as described herein and isolated from the spent (conditioned) culture medium. The compositions of the bioactive material primed viable placental tissue can be cryopreserved or lyophilized.

In some embodiments, the compositions of bioactive material primed viable placental tissue are partially depleted of, substantially free of, or free of one or more of viable immunogenic cells, trophoblasts, CD14+ macrophages, vascularized tissue-derived immunogenic cells, immunogenic maternal cells, maternal dendritic cells, or maternal leukocytes. In some embodiments, the compositions of bioactive material primed viable placental tissue comprise less than about 0.5%, or less than about 1%, or less than about 5% of CD14+ macrophages relative to the total number of native cells in the tissue. In some embodiments, the compositions of bioactive material primed viable placental tissue are partially depleted of, substantially free of, or free of one or more of vascularized tissue, blood vessels, blood, or trophoblast layer.

The cell viability of the native cells in the bioactive material primed viable placental tissue can be at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 90%. The native cells can be at least one of MSCs, epithelial cells, or fibroblasts. The cell viability is the proportion of live cells as compared to the number of total cells at a given time. Determination of the cell viability can be done using methods known to one of skill in the art. In some embodiments, the cell viability of the native cells in the bioactive material primed viable placental tissue is at least about 70%.

### 3. Compositions of Byproducts of Bioactive Material Priming

The spent culture medium resulting from the bioactive material priming process, which has become a conditioned medium, can be collected, and used for various purposes. During the bioactive material priming process, bioactive materials can be produced by the viable placental tissue. Some of these bioactive materials, which are byproducts of the bioactive material priming process, can remain in the bioactive material primed viable placental tissue and some of these bioactive materials can be deposited into the culture medium. Some of the bioactive materials can the same bioactive materials as those that were added to the culture medium and some can be different bioactive materials. Examples of these bioactive materials include, but are not limited to extracellular vesicles, exosomes, microvesicles, secretomes, cytokines, growth factors, peptides, antimicrobial peptides, and extracellular matrices. In some embodiments, the spent culture medium itself containing one or more bioactive materials can be used in regenerative medicine compositions. In other embodiments, one or more bioactive materials can be isolated from the spent culture medium by methods known to one of skill in the art. The byproducts of the bioactive material priming process, i.e., the spent (conditioned) culture medium and/or the bioactive materials therein, can be used in regenerative medicine compositions. Also disclosed herein are compositions of the spent (conditioned) medium resulting from the bioactive material priming of viable placental tissue and compositions of one or more bioactive materials isolated from the spent (conditioned) medium. The compositions can further comprise a pharmaceutically acceptable carrier. In some embodiments, the pharmaceutically acceptable carrier is aqueous based. In other embodiments, the pharmaceutically acceptable carrier is non-aqueous based. In still other embodiments, the pharmaceutically acceptable carrier is a powder. Examples of suitable pharmaceutically acceptable carriers include but are not limited to suspensions, solutions, gels, pastes, emulsions, creams, lotions, ointments, or powders. The pharmaceutically acceptable carriers can comprise excipients. These excipients include but are not limited to saline solutions, water, buffers, buffer solutions, sugars, trehalose, proteins, starches, emulsifiers, gelling agents, preservatives, antimicrobials, pH adjusters, and surfactants. The carriers can also comprise active pharmaceutical ingredients. The carriers can be formulated for various routes of administration including but not limited to topical, injection, or surgical, and can include coatings for medical devices or implants. The compositions can be cryopreserved or lyophilized.

### 4. Methods of Using Bioactive Material Primed Viable Placental Tissue Compositions and Byproduct Compositions

Also disclosed herein are methods of using bioactive material primed viable placental tissue and compositions thereof in regenerative medicine. Disclosed are methods of regenerating, replacing, or repairing diseased, damaged, or injured body tissue of a subject, the methods comprising administering to the subject any one of the bioactive material-primed viable placental tissue compositions disclosed herein.

Also disclosed herein are methods of using in regenerative medicine the compositions of the spent (conditioned) medium from the bioactive material priming of viable placental tissue and/or compositions of the byproducts of the bioactive material priming process found in and isolated from the spent (conditioned) medium. Disclosed are methods of regenerating, replacing, or repairing diseased, damaged, or injured body tissue of a subject, the methods comprising administering to the subject any one of the compositions of the spent (conditioned) medium from the bioactive material priming of viable placental tissue and/or compositions of the byproducts of the bioactive material priming process found in and isolated from the spent (conditioned) medium disclosed herein.

Examples of body tissue that can be diseased, damaged, or injured includes, but is not limited to tendons, cartilage, ligaments, periosteum, perichondrium, synovium, fascia, mesentery, sinew, dental tissue, gums, fistulas, nasal septum, vaginal wall tissue, abdominal wall tissue, peritoneum, tumor resection sites, dermal tissue, dermal wounds, dermal lesions, dermal abrasions, dermal burns, first degree burns, partial thickness burns, full thickness burns, epidermal wounds, congenital wounds, toxic epidermal necrolysis, epidermolysis bullosa, pyoderma gangrenosum, dermal ulcers, diabetic ulcers, diabetic foot ulcers, venous ulcers, venous let ulcers, pressure ulcers, arterial ulcers, decubitus ulcers, stasis ulcers, ischemic ulcers, chronic wounds, acute wounds, surgical wounds, internal wounds, hernia, bladder tissue, keloids, adhesions, organ lacerations, organ defects, diseased organ tissue, epithelial defects, spinal cord tissue, nerve tissue, tympanic membranes, and/or mucous membranes.

The compositions can be administered topically, subcutaneously, surgically, or by injection, e.g., intramuscular injection. The compositions can be administered by coating the composition onto the surface of a medical device implant and implanting the coated device into the subject.

### D. UV Light Plus Hypoxia Priming

### 1. UV Light Plus Hypoxia Priming Methods

Disclosed herein are methods for priming viable placental tissue by exposure to UV light and hypoxic conditions. The methods comprise (a) providing the viable placental tissue, wherein the viable placental tissue comprises viable cells native to the viable placental tissue, (b) exposing the viable placental tissue to UV light, and (c) contacting the viable placental tissue with a culture media at hypoxic conditions, thereby priming the viable placental tissue and generating spent culture medium. Optionally, (d) the primed viable placental tissue is separated from the spent culture medium. The viable placental tissue can be exposed to UV light before exposure to hypoxic conditions or the viable placental tissue can be exposed to hypoxic conditions before exposure to UV light. In some embodiments, the viable placental tissue is exposed to UV light before exposure to hypoxic conditions. The UV light plus hypoxia primed viable placental tissue after culturing can be separated from the spent (conditioned) medium or it can remain in contact with the spent (conditioned medium).

Prior to the UV light plus hypoxia priming process, the viable placental tissue can be collected and processed as described herein. In some embodiments, the viable placental tissue is amnion tissue, chorion tissue, Wharton's Jelly tissue, or umbilical cord tissue, or mixtures thereof. In some embodiments, the viable cells comprise one or more of mesenchymal stromal cells (MSCs), epithelial cells, or fibroblasts. The viable placental tissue can be in many types of forms such as sheets, wraps, grafts, pieces, minced pieces, crushed pieces, diced pieces, chopped pieces, cut pieces, chunks, or powder. In some embodiments, the viable placental tissue is in the form of a sheet, wrap, or graft. In other embodiments, the viable placental tissue is in the form of minced pieces, or a powder. The viable placental tissue can be cryopreserved and then thawed prior to priming. The viable placental tissue can be lyophilized prior to priming. The lyophilized viable placental tissue can be rehydrated (reconstituted) with water or an aqueous solution such as saline solution prior to priming.

The UV light region covers the wavelength range of 100-400 nm and is divided into three bands: UVA (315-400 nm), UVB (280-315 nm) and UVC (100-280 nm). The UV light can be UVA, UVB, or UVC or combinations thereof. In some embodiments, the UV light is UVB light. The source of the UV light can be from any suitable UV light source, such as a UV lamp. A suitable UV lamp is commercially available from ThermoFisher Scientific Cat #: 95034. The UV light as described herein is artificial and does not include ambient room lighting. The UV light source can emit UV light at a wavelength at any single wavelength within any of the UV wavelength band ranges of UVA, UVB, or UVC. In some embodiments, the UV light source emits a wavelength of about 302 nm. In some embodiments, the UV light source emits a wavelength of about 312 nm. In other embodiments, the UV light source emits a wavelength of about 252 nm. In still other embodiments, the UV light source emits a wavelength of about 365 nm. The wattage output of the UV light can be at wattage ranges as disclosed in UV Light Priming supra. In some embodiments, the wattage output of the UV light is about 8 watts.

The viable placental tissue sample can be positioned at a suitable distance away from the UV light source so the sample can be adequately exposed with UV light. For example, if the sample is a flat sheet or piece with a top and bottom surface, then one side of the sample can be exposed to UV light, or both sides of the sample can be exposed to UV light by exposing one side first and then flipping the sample over to expose the opposite side. In some embodiments, the distance is at distance ranges as disclosed in UV Light Priming supra.

The exposure time to the UV light is temporary and can be at time ranges as disclosed in UV Light Priming supra.

The priming under hypoxic conditions can take place in a hypoxia chamber or an incubator with the ability to control the atmospheric conditions inside the incubator at hypoxic conditions. The hypoxia chamber can be a self-contained and sealed chamber that fits inside existing laboratory incubators or can be a chamber/incubator with the ability to control the atmospheric conditions inside the chamber at hypoxic conditions. These hypoxia chambers and incubators are commercially available. The hypoxic conditions can include oxygen levels at levels as disclosed in Hypoxia Priming supra.

The hypoxia chamber/incubator atmosphere can be humidified during the priming process. The relative humidity (RH) level can be at levels as disclosed for Viable Placental Tissue supra. In some embodiments, the incubator is humidified at about 95% RH. The hypoxia chamber/incubator atmosphere can also contain CO₂ during the priming process. The CO₂ level can be from about 1% to about 10%, or from about 1% to about 7%, or from about 1% to about 5%, or from about 3% to about 7%, or from about 4% to about 6%, or about 5%. The hypoxia chamber/incubator can be at a temperature during the priming process of from about 20° C. to about 40° C., or from about 20° C. to about 30° C. or from about 25° C. to about 40° C., or from about 30° C. to about 40° C., or from about 35° C. to about 40° C., or from about 36° C. to about 38° C., or about 20° C., or about 25° C., or about 30° C., or about 35° C., or about 36° C., or about 37° C., or about 38° C., or about 39° C., or about 40° C. In some embodiments, the hypoxia chamber/incubator temperature is at about 37° C. during the priming process.

The exposure time for culturing under the hypoxic conditions can be at time ranges as disclosed for Hypoxia Priming supra.

The culture medium used for priming the tissue under hypoxic conditions can contain additives, nutrients, and/or antibiotics. The culture medium can be a commercially available cell culture medium with or without additives as known by one of skill in the art. A non-limiting example of a culture medium includes Dulbecco's Modified Eagle's Medium (DMEM) available from Sigma Aldrich. Non-limiting additives can be lysed platelets, antibiotics, fetal bovine serum (FBS), or human serum albumin. For example, the culture medium can be DMEM containing about 1% to about 10% lysed platelets, about 1% to about 10% FBS, or about 1% to about 10% human serum albumin. The culture medium can contain lysed platelets, FBS or human serum albumin at about 0.5%, or about 1%, or about 1.5%, or about 2%, or about 2.5%, or about 3%, or about 3.5%, or about 4%, or about 4.5%, or about 5%, or about 5.5%, or about 6%, or about 6.5%, or about 7%, or about 7.5%, or about 8%, or about 8.5%, or about 9%, or about 9.5%, or about 10%, or about 1% to about 10%, or about 1% to about 7%, or about 1% to about 5%, or about 1% to about 3%, or about 1% to about 2.5%. Another non-limiting example of a culture medium is commercially available chemically defined medium with or without serum. A non-limiting example of an antibiotic can be a penicillin-streptomycin solution such as Gibco™ pen-strep available from ThermoFisher Scientific. In some embodiments, the culture medium comprises DMEM. In some embodiments, the culture medium comprises nutrients. In some embodiments, the culture medium comprises serum. In some embodiments, the culture medium comprises one or more of lysed platelets, antibiotics, fetal bovine serum (FBS), or human serum albumin. In some embodiments, the lysed platelets are included in the culture medium at about 1% to about 10%. In some embodiments, the FBS is included in the culture medium at about 1% to about 10%. In some embodiments, the human serum albumin is included in the culture medium at about 1% to about 10%. In some embodiments, the culture medium comprises a chemically defined medium with or without serum. In some embodiments, the culture medium comprises DMEM containing about 2.5% FBS.

Bioactive materials can be added to the culture medium used for priming under hypoxic conditions. The addition of bioactive materials to the culture medium can contribute to the priming effects on the viable placental tissue. A bioactive material is a substance or compound having a biological effect upon a living organism, tissue, or cell. Examples of bioactive materials include, but are not limited to growth factors, peptides, antimicrobial peptides, cytokines, enzymes, extracellular vesicles, extracellular matrices, TNF-alpha, and interferon-gamma. In some embodiments, the one or more bioactive materials comprise TNF-alpha at an amount of about 5 to about 50 ng/ml, interferon-gamma at an amount of about 5 to about 50 ng/ml, or a combination of TNF-alpha at an amount of about 5 to about 50 ng/ml and interferon-gamma at an amount of about 5 to about 50 ng/ml. The bioactive materials can be in the form of nanoparticles. In some embodiments, the method further comprises adding an effective amount of one or more bioactive materials to the culture medium to be used in the priming process.

The resulting UV light plus hypoxia primed viable placental tissues can be amnion tissue, chorion tissue, or umbilical cord tissue, or mixtures thereof, and can be in many types of forms such as sheets, wraps, grafts, pieces, minced pieces, crushed pieces, diced pieces, chopped pieces, cut pieces, chunks, or powder. In some embodiments, the resulting UV light plus hypoxia primed viable placental tissue is in the form of a sheet, wrap, or graft. In other embodiments, the UV light plus hypoxia primed viable placental tissue is in the form of minced pieces, or a powder. The viable cells in the resulting UV light plus hypoxia primed viable placental tissue can include but are not limited to one or more of mesenchymal stromal cells (MSCs), epithelial cells, or fibroblasts.

As compared to a non-primed viable placental tissue, the UV plus hypoxia primed viable placental tissue can exhibit increased HBD-2 secretion of about a 1 fold to about a 10 fold increase, or about a 1 fold to about a 9 fold increase, or about a 1 fold to about a 8 fold increase, or about a 1 fold to about a 7 fold increase, or about a 1 fold to about a 6 fold increase, or about a 1 fold to about a 5 fold increase, or about a 1 fold to about a 4 fold increase, or about a 1 fold to about a 3 fold increase, or about a 1 fold to about a 2 fold increase, or about a 2 fold to about a 10 fold increase, or about a 2 fold to about a 9 fold increase, or about a 2 fold to about a 8 fold increase, or about a 2 fold to about a 7 fold increase, or about a 2 fold to about a 6 fold increase, or about a 2 fold to about a 5 fold increase, or about a 2 fold to about a 4 fold increase, or about a 2 fold to about a 3 fold increase, or about a 3 fold to about a 10 fold increase, or about a 3 fold to about a 9 fold increase, or about a 3 fold to about a 8 fold increase, or about a 3 fold to about a 7 fold increase, or about a 3 fold to about a 6 fold increase, or about a 3 fold to about a 5 fold increase, or about a 3 fold to about a 4 fold increase, or about a 4 fold to about a 10 fold increase, or about a 4 fold to about a 9 fold increase, or about a 4 fold to about a 8 fold increase, or about a 4 fold to about a 7 fold increase, or about a 4 fold to about a 6 fold increase, or about a 4 fold to about a 5 fold increase, or at least a 1 fold increase, or at least a 2 fold increase, or at least a 3 fold increase, or at least a 4 fold increase, or at least a 5 fold increase, or at least a 6 fold increase, or at least a 7 fold increase, or at least a 8 fold increase, or at least a 9 fold increase, or at least a 10 fold increase; and/or can exhibit enhanced VEGF-A function as determined by a cellular functional assay of about a 1 fold to about a 10 fold increase in activity, or about a 1 fold to about a 9 fold increase in activity, or about a 1 fold to about a 8 fold increase in activity, or about a 1 fold to about a 7 fold increase in activity, or about a 1 fold to about a 6 fold increase in activity, or about a 1 fold to about a 5 fold increase in activity, or about a 1 fold to about a 4 fold increase in activity, or about a 1 fold to about a 3 fold increase in activity, or about a 1 fold to about a 2 fold increase in activity, or about a 2 fold to about a 10 fold increase in activity, or about a 2 fold to about a 9 fold increase in activity, or about a 2 fold to about a 8 fold increase in activity, or about a 2 fold to about a 7 fold increase in activity, or about a 2 fold to about a 6 fold increase in activity, or about a 2 fold to about a 5 fold increase in activity, or about a 2 fold to about a 4 fold increase in activity, or about a 2 fold to about a 3 fold increase in activity, or about a 3 fold to about a 10 fold increase in activity, or about a 3 fold to about a 9 fold increase in activity, or about a 3 fold to about a 8 fold increase in activity, or about a 3 fold to about a 7 fold increase in activity, or about a 3 fold to about a 6 fold increase in activity, or about a 3 fold to about a 5 fold increase in activity, or about a 3 fold to about a 4 fold increase in activity, or about a 4 fold to about a 10 fold increase in activity, or about a 4 to about a 9 fold increase in activity, or about a 4 to about a 8 fold increase in activity, or about a 4 to about a 7 fold increase in activity, or about a 4 to about a 6 fold increase in activity, or about a 4 to about a 5 fold increase in activity, or about a 5 fold to about a 10 fold increase in activity, or about a 5 fold to about a 9 fold increase in activity, or about a 5 fold to about a 8 fold increase in activity, or about a 5 fold to about a 7 fold increase in activity, or about a 5 fold to about a 6 fold increase in activity, or at least a 1 fold increase in activity, or at least a 2 fold increase in activity, or at least a 3 fold increase in activity, or at least a 4 fold increase in activity, or at least a 5 fold increase in activity, or at least a 6 fold increase in activity, or at least a 7 fold increase in activity, or at least a 8 fold increase in activity, or at least a 9 fold increase in activity, or at least a 10 fold increase in activity and/or can exhibit increased VEGF-A secretion of about a 1 fold to about a 10 fold increase, or about a 1 fold to about a 9 fold increase, or about a 1 fold to about a 8 fold increase, or about a 1 fold to about a 7 fold increase, or about a 1 fold to about a 6 fold increase, or about a 1 fold to about a 5 fold increase, or about a 1 fold to about a 4 fold increase, or about a 1 fold to about a 3 fold increase, or about a 1 fold to about a 2 fold increase, or about a 2 fold to about a 10 fold increase, or about a 2 fold to about a 9 fold increase, or about a 2 fold to about a 8 fold increase, or about a 2 fold to about a 7 fold increase, or about a 2 fold to about a 6 fold increase, or about a 2 fold to about a 5 fold increase, or about a 2 fold to about a 4 fold increase, or about a 2 fold to about a 3 fold increase, or about a 3 fold to about a 10 fold increase, or about a 3 fold to about a 9 fold increase, or about a 3 fold to about a 8 fold increase, or about a 3 fold to about a 7 fold increase, or about a 3 fold to about a 6 fold increase, or about a 3 fold to about a 5 fold increase, or about a 3 fold to about a 4 fold increase, or at least a 1 fold increase, or at least a 2 fold increase, or at least a 3 fold increase, or at least a 4 fold increase, or at least a 5 fold increase, or at least a 6 fold increase, or at least a 7 fold increase, or at least a 8 fold increase, or at least a 9 fold increase, or at least a 10 fold increase; and/or can exhibit increased IL-1RA secretion of about a 1 fold to about a 10 fold increase, or about a 1 fold to about a 9 fold increase, or about a 1 fold to about a 8 fold increase, or about a 1 fold to about a 7 fold increase, or about a 1 fold to about a 6 fold increase, or about a 1 fold to about a 5 fold increase, or about a 1 fold to about a 4 fold increase, or about a 1 fold to about a 3 fold increase, or about a 1 fold to about a 2 fold increase, or about a 2 fold to about a 10 fold increase, or about a 2 fold to about a 9 fold increase, or about a 2 fold to about a 8 fold increase, or about a 2 fold to about a 7 fold increase, or about a 2 fold to about a 6 fold increase, or about a 2 fold to about a 5 fold increase, or about a 2 fold to about a 4 fold increase, or about a 2 fold to about a 3 fold increase, or about a 3 fold to about a 10 fold increase, or about a 3 fold to about a 9 fold increase, or about a 3 fold to about a 8 fold increase, or about a 3 fold to about a 7 fold increase, or about a 3 fold to about a 6 fold increase, or about a 3 fold to about a 5 fold increase, or about a 3 fold to about a 4 fold increase, or at least a 1 fold increase, or at least a 2 fold increase, or at least a 3 fold increase, or at least a 4 fold increase, or at least a 5 fold increase, or at least a 6 fold increase, or at least a 7 fold increase, or at least a 8 fold increase, or at least a 9 fold increase, or at least a 10 fold increase. In some embodiments, the UV light plus hypoxia primed viable placental tissue exhibits one or more of the following increased therapeutic regenerative properties comprising an increase in HBD-2 secretion (preferably about a 1 fold to about a 4 fold increase); enhanced angiogenic factor (VEGF-A) function (preferably about a 3 fold to about a 5 fold increase in activity) as determined by a cellular functional assay; an increase in VEGF-A secretion (preferably about a 1 fold to about a 3 fold increase); and/or an increase in IL-1RA secretion preferably about a 1 fold to about a 3 fold increase) as compared to viable placental tissue not exposed to hypoxic conditions and UV light (non-primed).

The methods can further comprise collecting the spent culture medium of step (d) and isolating from the spent culture medium one or more bioactive materials formed in the culture medium during step (b) and (c). The isolated one or more bioactive materials can include, but is not limited to extracellular vesicles, exosomes, microvesicles, secretomes, cytokines, growth factors, peptides, antimicrobial peptides, or extracellular matrices.

The cell viability of the native cells in the viable placental tissue after UV light plus hypoxia priming can be at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 90%. The native cells can be at least one of MSCs, epithelial cells, or fibroblasts. The cell viability is the proportion of live cells as compared to the number of total cells at a given time. Determination of the cell viability can be done using methods known to one of skill in the art. In some embodiments, the cell viability of the native cells in the viable placental tissue after UV light plus hypoxia priming is at least about 70%.

The UV light plus hypoxia primed viable placental tissue can further be cryopreserved or lyophilized after the priming process and can be stored in suitable packaging. The cryopreserved UV light plus hypoxia primed viable placental tissue can be thawed prior to use. The lyophilized UV light plus hypoxia primed viable placental tissue can be rehydrated (reconstituted) with water or an aqueous solution such as saline solution prior to use or used as is.

### 2. Compositions of UV Light Plus Hypoxia Primed Viable Placental Tissue

Also disclosed herein are compositions comprising UV light plus hypoxia primed viable placental tissues. In some embodiments the compositions comprise UV light plus hypoxia primed viable placental tissues produced by any of the UV light plus hypoxia priming methods described herein. In some embodiments, the UV light plus hypoxia primed viable placental tissue was primed by exposure of the tissues to UV light and hypoxic conditions. In some embodiments, the UV light was UVA light, or UVB light, or combinations thereof. In some embodiments, the UV light was UVB light. In some embodiments, the hypoxic conditions were from about 1% to about 5% O₂ or from about 1% to about 2% O₂. The UV light plus hypoxia primed viable placental tissue can be amnion tissue, chorion tissue, Wharton's Jelly tissue, or umbilical cord tissue, or mixtures thereof. In some embodiments, the viable cells of the UV light plus hypoxia primed viable placental tissue comprise one or more of mesenchymal stromal cells (MSCs), epithelial cells, or fibroblasts. In some embodiments, the UV light plus hypoxia primed viable placental tissue exhibits one or more of the following increased therapeutic regenerative properties comprising an increase in HBD-2 secretion (preferably about a 1 fold to about a 4 fold increase); enhanced angiogenic factor (VEGF-A) function (preferably about a 3 fold to about a 5 fold increase in activity) as determined by a cellular functional assay; an increase in VEGF-A secretion (preferably about a 1 fold to about a 3 fold increase); and/or an increase in IL-1RA secretion (preferably about a 1 fold to about a 3 fold increase) as compared to viable placental tissue not exposed to hypoxic conditions and UV light (non-primed). The UV light plus hypoxia primed viable placental tissue can be in many types of forms such as sheets, wraps, grafts, pieces, minced pieces, crushed pieces, diced pieces, chopped pieces, cut pieces, chunks, or powder. In some embodiments, the UV light plus hypoxia primed viable placental tissue is in the form of a sheet, wrap, or graft. In other embodiments, the UV light plus hypoxia primed viable placental tissue is in the form of minced pieces, or a powder.

The compositions of UV light plus hypoxia primed viable placental tissue can further comprise a pharmaceutically acceptable carrier. In some embodiments, the pharmaceutically acceptable carrier is aqueous based. In other embodiments, the pharmaceutically acceptable carrier is non-aqueous based. In still other embodiments, the pharmaceutically acceptable carrier is a powder. Examples of suitable pharmaceutically acceptable carriers include but are not limited to suspensions, solutions, gels, pastes, emulsions, creams, lotions, ointments, or powders. The pharmaceutically acceptable carriers can comprise excipients that will not cause significant damage to the viability of the cells. These excipients include but are not limited to saline solutions, water, buffers, buffer solutions, sugars, trehalose, proteins, starches, emulsifiers, gelling agents, preservatives, antimicrobials, pH adjusters, and surfactants. The carriers can also comprise active pharmaceutical ingredients. The carriers can be formulated for various routes of administration including but not limited to topical, injection, or surgical, and can include coatings for medical devices or implants.

The compositions of UV light plus hypoxia primed viable placental tissue can further comprise one or more bioactive materials including but not limited to extracellular vesicles, exosomes, microvesicles, secretomes, cytokines, growth factors, peptides, antimicrobial peptides, extracellular matrices, TNF-alpha (preferably at an amount from about 5 to about 50 ng/ml), interferon-gamma (preferably at an amount from about 5 to about 50 ng/ml), or nanoparticles thereof. In some embodiments, the bioactive materials comprise one or more of the bioactive materials produced by any of the UV light plus hypoxia priming methods of viable placental tissue as described herein and isolated from the spent (conditioned) culture medium. The compositions of the UV light plus hypoxia primed viable placental tissue can be cryopreserved or lyophilized.

In some embodiments, the compositions of UV light plus hypoxia primed viable placental tissue are partially depleted of, substantially free of, or free of one or more of viable immunogenic cells, trophoblasts, CD14+ macrophages, vascularized tissue-derived immunogenic cells, immunogenic maternal cells, maternal dendritic cells, or maternal leukocytes. In some embodiments, the compositions of UV light plus hypoxia primed viable placental tissue comprise less than about 0.5%, or less than about 1%, or less than about 5% of CD14+ macrophages relative to the total number of native cells in the tissue. In some embodiments, the compositions of UV light plus hypoxia primed viable placental tissue are partially depleted of, substantially free of, or free of one or more of vascularized tissue, blood vessels, blood, or trophoblast layer.

The cell viability of the native cells in the UV light plus hypoxia primed viable placental tissue can be at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 90%. The native cells can be at least one of MSCs, epithelial cells, or fibroblasts. The cell viability is the proportion of live cells as compared to the number of total cells at a given time. Determination of the cell viability can be done using methods known to one of skill in the art. In some embodiments, the cell viability of the native cells in the UV light plus hypoxia viable placental tissue is at least about 70%.

### 3. Compositions of Byproducts of UV Light Plus Hypoxia Primed Viable Placental Tissue

The spent culture medium resulting from the UV light plus hypoxia priming process, which has become a conditioned medium and is a byproduct of the UV light plus hypoxia priming process, can be collected, and used for various purposes. During the UV light plus hypoxia priming process, bioactive materials can be produced by the viable placental tissue. Some of these bioactive materials, which are also byproducts of the UV light plus hypoxia priming process, can remain in the UV light plus hypoxia primed viable placental tissue and some of these bioactive materials can be deposited into the culture medium. If any bioactive materials were added to the culture medium used for the UV light plus hypoxia priming, some of the bioactive materials that are produced by the viable placental tissue can the same bioactive materials as those that were added, and some can be different bioactive materials from those that were added. Examples of these bioactive materials that are formed during the UV light plus hypoxia priming process include, but are not limited to extracellular vesicles, exosomes, microvesicles, secretomes, cytokines, growth factors, peptides, antimicrobial peptides, and extracellular matrices. In some embodiments, the spent culture medium itself containing one or more bioactive materials can be used in regenerative medicine compositions. In other embodiments, one or more bioactive materials can be isolated from the spent culture medium by methods known to one of skill in the art. The byproducts of the UV light plus hypoxia priming process, i.e., the spent (conditioned) culture medium and/or the bioactive materials therein, can be used in regenerative medicine compositions. Also disclosed herein are compositions of the spent (conditioned) medium resulting from the UV light plus hypoxia priming of viable placental tissue and compositions of one or more bioactive materials isolated from the spent (conditioned) medium. The compositions can further comprise a pharmaceutically acceptable carrier. In some embodiments, the pharmaceutically acceptable carrier is aqueous based. In other embodiments, the pharmaceutically acceptable carrier is non-aqueous based. In still other embodiments, the pharmaceutically acceptable carrier is a powder. Examples of suitable pharmaceutically acceptable carriers include but are not limited to suspensions, solutions, gels, pastes, emulsions, creams, lotions, ointments, or powders. The pharmaceutically acceptable carriers can comprise excipients. These excipients include but are not limited to saline solutions, water, buffers, buffer solutions, sugars, trehalose, proteins, starches, emulsifiers, gelling agents, preservatives, antimicrobials, pH adjusters, and surfactants. The carriers can also comprise active pharmaceutical ingredients. The carriers can be formulated for various routes of administration including but not limited to topical, injection, or surgical, and can include coatings for medical devices or implants. The compositions can be cryopreserved or lyophilized.

### 4. Methods of Using UV Light Plus Hypoxia Primed Viable Placental Tissue Compositions and Byproduct Compositions

Also disclosed herein are methods of using UV light plus hypoxia primed viable placental tissue and compositions thereof in regenerative medicine. Disclosed are methods of regenerating, replacing, or repairing diseased, damaged, or injured body tissue of a subject, the methods comprising administering to the subject any one of the UV light plus hypoxia primed viable placental tissue compositions disclosed herein.

Also disclosed herein are methods of using in regenerative medicine the compositions of the spent (conditioned) medium from the UV light plus hypoxia priming of viable placental tissue and/or compositions of the byproducts of the UV light plus hypoxia priming process found in and isolated from the spent (conditioned) medium. Disclosed are methods of regenerating, replacing, or repairing diseased, damaged, or injured body tissue of a subject, the methods comprising administering to the subject any one of the compositions of the spent (conditioned) medium from the UV light plus hypoxia priming of viable placental tissue and/or compositions of the byproducts of the UV light plus hypoxia priming process found in and isolated from the spent (conditioned) medium disclosed herein.

Examples of body tissue that can be diseased, damaged, or injured includes, but is not limited to tendons, cartilage, ligaments, periosteum, perichondrium, synovium, fascia, mesentery, sinew, dental tissue, gums, fistulas, nasal septum, vaginal wall tissue, abdominal wall tissue, peritoneum, tumor resection sites, dermal tissue, dermal wounds, dermal lesions, dermal abrasions, dermal burns, first degree burns, partial thickness burns, full thickness burns, epidermal wounds, congenital wounds, toxic epidermal necrolysis, epidermolysis bullosa, pyoderma gangrenosum, dermal ulcers, diabetic ulcers, diabetic foot ulcers, venous ulcers, venous let ulcers, pressure ulcers, arterial ulcers, decubitus ulcers, stasis ulcers, ischemic ulcers, chronic wounds, acute wounds, surgical wounds, internal wounds, hernia, bladder tissue, keloids, adhesions, organ lacerations, organ defects, diseased organ tissue, epithelial defects, spinal cord tissue, nerve tissue, tympanic membranes, and/or mucous membranes.

The compositions can be administered topically, subcutaneously, surgically, or by injection, e.g., intramuscular injection. The compositions can be administered by coating the composition onto the surface of a medical device implant and implanting the coated device into the subject.

### III. Containers

Compositions of the present invention can also be included in a container. The container can include a bottle, a metal tube, a laminate tube, a plastic tube, a dispenser, a pressurized container, a barrier container, a package, a compartment, etc. In certain aspects, the container can include plastic or metal foil or a combination thereof. In certain aspects, the container can be hermetically sealed.

In one particular aspect, the container can include a support assembly for supporting a composition of the present invention. The support assembly can have a base and a cover. The base can have a longitudinal axis and comprise a receiving portion that can include the composition. The receiving portion can have a top surface and an opposed bottom surface that are spaced apart relative to a vertical axis that is perpendicular to the longitudinal axis of the base. The receiving portion can have at least one traction-creating feature. The traction-creating feature can include a rough top surface and/or a plurality of perforations that extend between the top and bottom surfaces of the receiving portion. The cover can have a longitudinal axis, a top surface, and an opposed bottom surface. The cover can be configured for releasable coupling (optionally, attachment) to the base in a product-covering position. In the product-covering position, the cover overlies the receiving portion of the base. The base and the cover can be configured to cooperate to support a composition of the present invention. In certain aspects, a composition of the present invention can be positioned to contact at least a portion of the top surface of the product receiving portion of the base and at least a portion of the bottom surface of the cover. In some aspects, the support assemble can include features described in U.S. Pat. No. 10,279,974, which is incorporated into the present application by reference.

The container can include indicia on its surface. The indicia, for example, can be a word, a phrase, an abbreviation, a picture, or a symbol. In addition to a composition of the present invention, the container can also include instructions. The instructions can include, for example, an explanation of how to apply, use, and/or maintain the composition.

### EXAMPLES

### Example 1—Hypoxia Primed Viable Placental Tissue

Viable Placental Tissue Preparation: Viable human placentas from eligible donors were purchased from The National Disease Research Interchange (NDRI, Philadelphia, PA). The placentas were shipped overnight in cold storage. The placentas were aseptically processed and cleaned using blunt dissection techniques. The viable amnion membrane tissue (AM) and viable chorion membrane tissue (CM) were removed from the decidua and cleaned of other maternal tissue including of blood and trophoblast. The CM and AM were then stored overnight in 250 mL of DMEM low glucose+5% FBS+penicillin-streptomycin solution (pen-strep) (Gibco) in a 37° C. incubator, at 95% RH and 5% CO₂.

Tissue Priming: On the following day, CM and AM from three donors were cut into 5×5 cm dimension pieces using a scalpel and template. Six 5×5 cm pieces of each tissue were placed in a 10 cm cell culture dish and resuspended in 12 mL of DMEM+2.5% FBS. One dish from each representative tissue was placed in an incubator at 37° C. at normoxic conditions (20% to 21% 02) plus 5% CO₂ and 95% RH as controls and another dish was placed in an incubator at 37° C. at 2% 02 (hypoxia) plus 5% CO₂ and 95% RH. After 48 hours, the dishes were removed from the incubators, and the tissue pieces and spent culture medium were collected separately.

Tissue Lysis: Each 5×5 cm tissue piece was placed into a 2 mL Eppendorf tube and chopped roughly using fine scissors. To the tube 1 mL of T-PER (Thermo Fisher)+Protease Inhibitor (Roche) was added to aid in tissue and cell lysis. To the tube one 5 mm steel bead (Qiagen) was added and then the tube was placed in a Qiagen TissueLyser LT and disrupted at maximum speed for 2 minutes, in three total cycles. The tubes were then centrifuged at 14000 g and the supernatant lysate was collected into another tube and frozen at minus 80° C. until assayed.

Multi-plex Analysis: The tissue lysates and supernatants were assayed using Multiplex (R&D Systems) assay kit containing VEGF-A and IL-1RA. The assay was processed as per standard kit protocol and read using Luminex® Magpix® multiplex reader.

VEGF Reporter Bioassay: VEGF reporter bioassay (Promega) was performed as per manufacturer instructions and read using a BioTek® Synergy™ HTX plate reader. Briefly, the standard and supernatants were added to their respective wells and then assay cells were thawed and added to each well. After six hours of incubation, the luciferase substrate reagent was added to quantify the functional VEGF present in the medium based on the standard curve.

Data and Statistical Analysis: Data were presented as mean±SE of representative experiments. Differences between groups were analyzed by independent t-test. P value less than 0.05 were considered as statistically significant.

Results and Discussion: To test whether hypoxia tissue priming will result in enhanced regenerative properties, human placental amnion membrane tissue (AM) and human placental chorion membrane tissue (CM) were treated for 48 hours in a hypoxic incubator with 2% oxygen in DMEM+2.5% FBS as described above. Tissues incubated in a normoxic incubator (about 20% to about 21% oxygen) were used as control. The resulting tissue and supernatant were then collected and quantified using a panel of key growth factors and cytokines by multiplex.

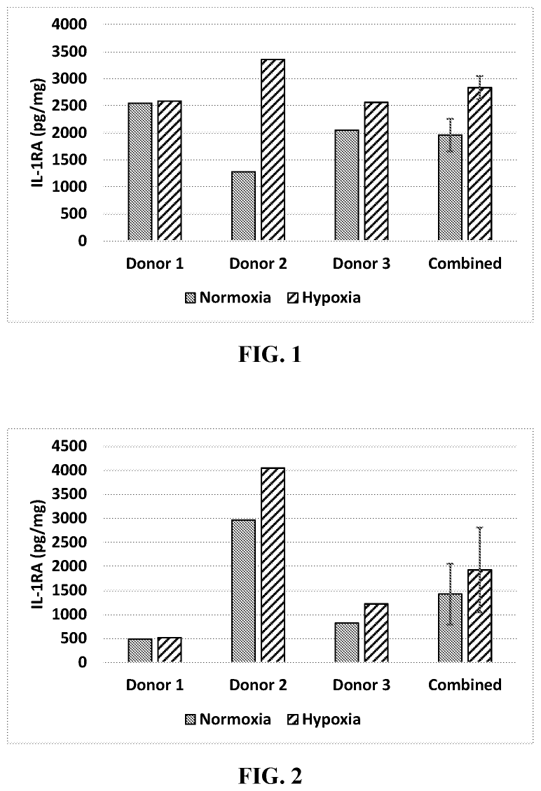

As a result of hypoxia priming, the secretions of IL-1RA, an anti-inflammatory factor, in AM and CM, and VEGF-A in CM were greatly boosted. On average, the IL-1RA secretion of AM for 48 hours in normoxic conditions was 1958.8 pg/mg total protein and 2834.1 pg/mg total protein for hypoxic conditions. Though there was donor variability, on average this correlated to a 1.45-fold increase in the secretion of IL-1RA in AM due to hypoxic treatment (FIG. 1). CM showed a similar trend as IL-1RA secretion levels were at 1423.7 pg normoxic and 1926.7 pg hypoxic, a 1.35-fold increase due to priming (FIG. 2). Therefore, viable AM and CM tissue primed using hypoxia is shown to boost IL-1RA levels over non-primed viable AM and CM tissue.

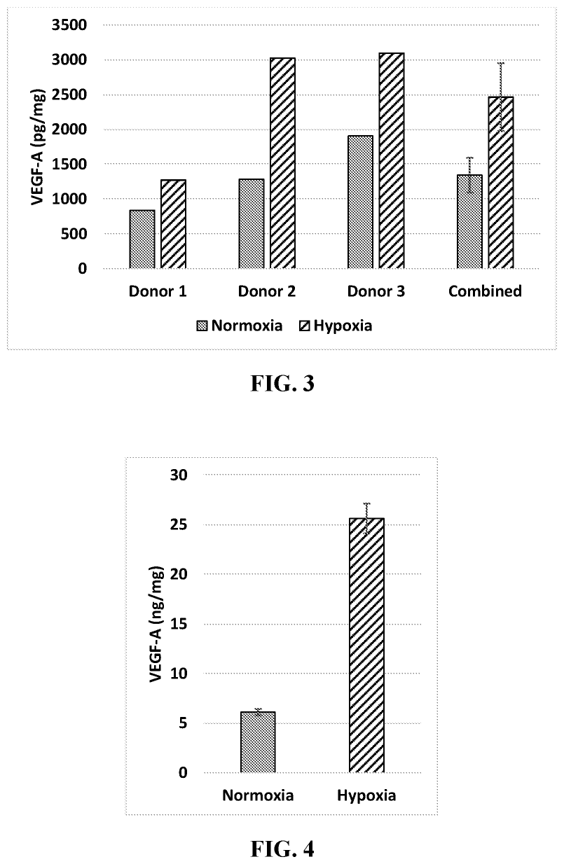

It was also found that hypoxia priming increased the level of VEGF-A. On average, the VEGF-A secretion of CM for 48 hours in normoxic conditions was 1344.9 pg/mg total protein and in hypoxic conditions the secretion was increased to 2466.5 pg/mg total protein. Though there was donor variability, this corresponded to a 1.83-fold increase in secretion of VEGF-A due to hypoxic treatment (FIG. 3). To ensure that the increased protein level indeed correlated with increased functionality, we carried out an in vitro cell-based functional assay (Promega). Using the collected cell medium from one of three CM donors, we observed a 4.2-fold increase in VEGF-A activity in the hypoxia group as compared to normoxia (FIG. 4).

Taken together, the study shows that hypoxia primed human placental amnion and human placental chorion tissues secrete increased levels of angiogenic factor, VEGF-A and anti-inflammatory factor, IL-1RA. The increased angiogenic factor is also associated with enhanced angiogenic function by an in vitro VEGF reporter assay. Overall, the results of the study suggest that hypoxia priming human viable placental tissue is an effective method to boost the factors associated with regenerative functions.

### Example 2—UV Light Primed Viable Placental Tissue

Viable Placental Tissue Preparation: Viable human placentas from eligible donors were purchased from The National Disease Research Interchange (NDRI, Philadelphia, PA). The placentas were shipped overnight in cold storage. The placentas were aseptically processed and cleaned using blunt dissection techniques. The viable amnion membrane tissue (AM) and viable chorion membrane tissue (CM) were removed from the decidua and cleaned of other maternal tissue including of blood and trophoblast. The CM and AM were then stored overnight in 250 mL of DMEM low glucose+5% FBS+penicillin-streptomycin solution (pen-strep) (Gibco) in a 37° C. incubator, at 95% RH and 5% CO₂.

Tissue Priming: On the following day, CM and AM from three donors were cut into 5×5 cm dimension pieces using a scalpel and template. Then, both AM and CM tissues were assigned into 4 groups. For AM, the four groups were named as control group, UVB 20-second group, UVB 40-second group, and UVB 60-second group. For CM, the four groups were named as control group, UVB 30-second group, UVB 60-second group, and UVB 180-second group. The tissues were then exposed to an UV Lamp with 302 nm wavelength at 8-watt output (ThermoFisher Scientific, Cat #: 95034) at a 10-cm distance for 20, 40, 60 seconds (AM) or 30, 60, 180 seconds (CM). The control groups were not exposed to UV light (non-primed). Three 5×5 cm pieces from each group were placed in a 10 cm cell culture dish and resuspended in 6 mL of DMEM+2.5% FBS. Then all the dishes containing tissues were placed in a 37° C. incubator at 20% 02 (normoxia) plus 5% CO₂ and 95% RH. After 48 hours, the dishes were removed from the incubator, and the tissue pieces and spent culture medium were collected separately.

Tissue Lysis: Each 5×5 cm tissue piece was placed into a 2 mL Eppendorf tube and chopped roughly using fine scissors. To the tube 1 mL of T-PER (Thermo Fisher)+Protease Inhibitor (Roche) was added to aid in tissue and cell lysis. To the tube one 5 mm steel bead (Qiagen) was added and then the tube was placed in a Qiagen TissueLyser LT and disrupted at maximum speed for 2 minutes, in three total cycles. The tubes were then centrifuged at 14000 g and the supernatant of the lysate was collected into another tube and frozen at minus 80° C. until assayed.

ELISA: The tissue lysates and supernatants were assayed using HBD-2 ELISA assay kit (Mybiosource, Cat #: MBS9314447). The assay was processed as per standard kit protocol and read using plate reader.

Data and Statistical Analysis: Data were presented as mean±SE of representative experiments. Differences between groups were analyzed by independent t-test. P value less than 0.05 were considered as statistically significant.

Results: To test whether tissue primed with UVB irradiation will result in increased antimicrobial peptide secretion, human placental amnion membrane tissue (AM) was exposed to UVB lamp for 20 seconds, 40 seconds, or 60 seconds and human placental chorion membrane tissue (CM) was exposed to UVB lamp for 30 seconds, 60 seconds, or 180 seconds as described above. Untreated, non-exposed tissues (non-primed) were used as control. After 48 hours of incubation, the resulting tissue and supernatant were then collected and the levels of antimicrobial peptide, HBD-2 were quantified by ELISA.

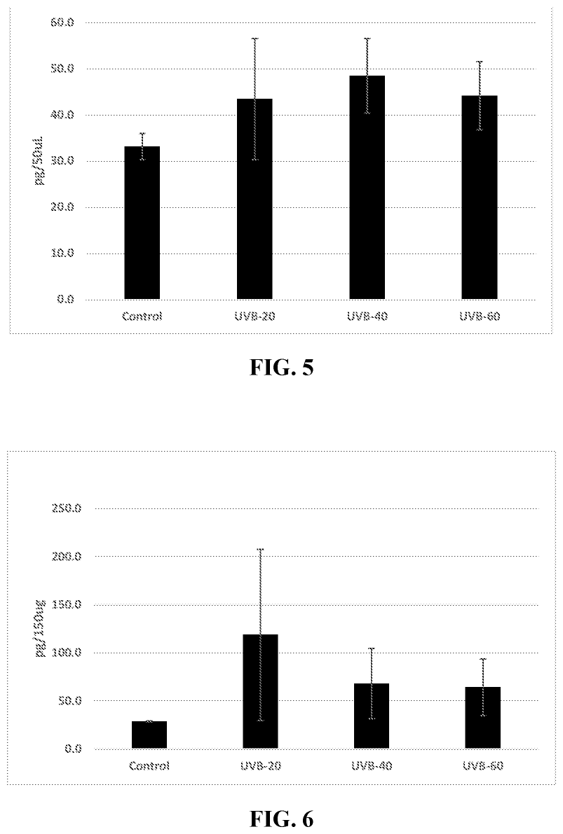

Regarding AM, comparing to control group, UVB irradiation increased HBD-2 secretion in the conditioned medium about 35-40% in all the treatment groups regardless of the irradiation doses (FIG. 5). Interestingly, the HBD-2 levels are much higher in the tissue lysates than in the conditioned medium (FIG. 6). The HBD-2 levels are increased about 2-fold to 4-fold after UVB irradiation in the tissue lysates, suggesting the majority of HBD-2 were remaining in the tissues rather than were released into the medium. Notably, a dose dependent response in both AM tissue lysates and AM conditioned medium was not seen.

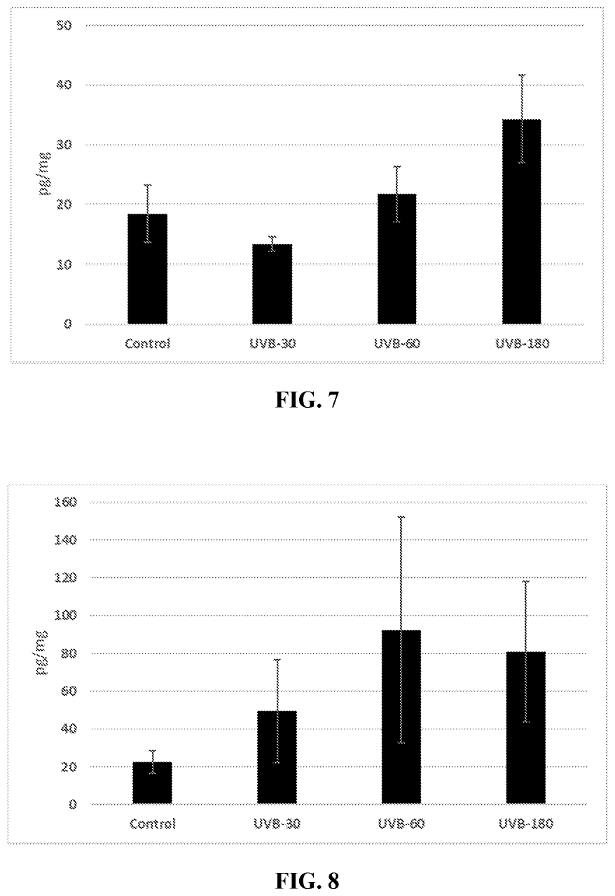

For CM samples, UVB irradiation increased HBD-2 secretion in the conditioned medium about 39% in the 60-second treatment group and about 91% increase in the 180-second treatment group (FIG. 7). Similarly, in CM tissue lysates, a 1.8-fold, 3.1-fold, and 3.2-fold increase in 30-second, 60-second, and 180-second treatment groups respectively was seen (FIG. 8). In addition, there seems to be a correlation between irradiation doses and the levels of HBD-2 secretion in both CM conditioned medium and CM tissue lysates.

The data show that irradiation of placental tissues by UVB increased the antimicrobial peptide HBD-2 secretion. Priming by UVB irradiation can therefore be a method to enhance the antimicrobial properties of viable placental tissues for use in regenerative medicine.

### Example 3—Bioactive Material Primed Viable Placental Tissue

Viable Placental Tissue Preparation: Viable human placentas from eligible donors will be obtained and shipped in cold storage. The placentas will be aseptically processed and cleaned using blunt dissection techniques. The viable amnion membrane tissue (AM) and viable chorion membrane tissue (CM) will be removed from the decidua and cleaned of other maternal tissue including of blood and trophoblast. The CM and AM will then be stored for 4 to 96 hours in DMEM low glucose+5% FBS+antibiotic solution in a 37° C. incubator, at 95% RH and 5% CO₂.

Tissue Priming: The CM and AM will be cut into pieces using a scalpel and template. The pieces of each tissue will be placed in a cell culture dish and resuspended in DMEM+2.5% FBS. One dish from each representative tissue will placed in an incubator at 37° C. at 5% CO₂ and 95% RH as controls. TNF-alpha at an amount of 10 ng/ml, interferon-gamma at an amount of 10 ng/ml, and/or a combination of TNF-alpha at an amount of 10 ng/ml and interferon-gamma at an amount of 10 ng/ml will be added to the culture medium in the other dish from each representative tissue and will be placed in an incubator at 37° C. at 5% CO₂ and 95% RH. After 4 to 96 hours, the dishes will be removed from the incubators, and the tissue pieces and spent culture medium will be collected separately.

Tissue Lysis: Each tissue piece will be placed into a 2 mL Eppendorf tube and chopped roughly using fine scissors. To the tube 1 mL of T-PER (Thermo Fisher)+Protease Inhibitor (Roche) will be added to aid in tissue and cell lysis. To the tube one 5 mm steel bead (Qiagen) will be added and then the tube will be placed in a Qiagen TissueLyser LT and disrupted at maximum speed for 2 minutes, in three total cycles. The tubes will then be centrifuged at 14000 g and the supernatant lysate will be collected into another tube and frozen at minus 80° C. until assayed.

Analysis: ELISA: The tissue lysates and supernatants will be assayed using PGE-2 ELISA assay kit. The assay will be processed as per standard kit protocol and read using plate reader. Multi-plex Analysis: The tissue lysates and supernatants will be assayed using Multiplex (R&D Systems) assay kit containing a group of key factors involved in regenerative properties. The assay will be processed as per standard kit protocol and read using Luminex® Magpix® multiplex reader.

To test whether bioactive material tissue priming of viable placental tissue will result in enhanced regenerative properties, human placental amnion membrane tissue (AM) and human placental chorion membrane tissue (CM) will be treated in an incubator with DMEM+2.5% FBS+the bioactive materials TNF-alpha, interferon-gamma or a combination of TNF-alpha and interferon-gamma, as described above. Tissues incubated in the culture medium without the added bioactive materials (non-primed) will be used as controls. The resulting tissue and supernatant will then be collected and analyzed.

The results will show that bioactive material primed viable placental tissue has enhanced therapeutic regenerative properties such as angiogenesis, anti-inflammatory, chemoattractant, antimicrobial, antioxidant or antifibrosis as compared to non-primed viable placental tissue as determined in vitro such as with ELISA and/or Multi-plex analysis, and/or in vivo as compared to non-primed viable placental tissue.

### Example 4—UV Light Plus Hypoxia Primed Viable Placental Tissue

Viable Placental Tissue Preparation: Viable human placentas from eligible donors will be obtained and shipped in cold storage. The placentas will be aseptically processed and cleaned using blunt dissection techniques. The viable amnion membrane tissue (AM) and viable chorion membrane tissue (CM) will be removed from the decidua and cleaned of other maternal tissue including of blood and trophoblast. The CM and AM will then be stored for 4 to 96 hours in DMEM low glucose+5% FBS+antibiotic solution in a 37° C. incubator, at 95% RH and 5% CO₂.

Tissue Priming: CM and AM will be cut into pieces using a scalpel and template. Then, both AM and CM tissues will be assigned into groups for certain exposure times to UVB light. A control group will not be exposed to UV light or hypoxic conditions. The exposure times will be selected from 5 seconds, 10 seconds, 15 seconds, 20 seconds, 25 seconds, 30 seconds, 35 seconds, 40 seconds, 45 seconds, 50 seconds, 55 seconds, 60 seconds, 70 seconds, 80 seconds, 90 seconds, 100 seconds, 110 seconds, 120 seconds, 130 seconds, 140 seconds, 150 seconds, 160 seconds, 170 seconds, 180 seconds, 3½ minutes, 4 minutes, 4½ minutes, 5 minutes, 6 minutes, 7 minutes, 8 minutes, 9 minutes, or 10 minutes. The tissues will then be exposed to an UV Lamp with 302 nm wavelength at 8-watt output (ThermoFisher Scientific, Cat #: 95034) at a 10-cm distance for the selected exposure times. Pieces from each group will be placed in a cell culture dish and resuspended in DMEM+2.5% FBS. Then the dishes containing tissues will be placed in a 37° C. incubator at 2% 02 (hypoxia) plus 5% CO₂ and 95% RH. After 4-96 hours, the dishes will be removed from the incubator, and the tissue pieces and spent culture medium will be collected separately. The control group will not be exposed to UV light or hypoxic conditions but will be cultured in an incubator at 37° C. at 20% 02 (normoxia) plus 5% CO₂ and 95% RH for 4-96 hours.

Tissue Lysis: Each tissue piece will be placed into a 2 mL Eppendorf tube and chopped roughly using fine scissors. To the tube 1 mL of T-PER (Thermo Fisher)+Protease Inhibitor (Roche) will be added to aid in tissue and cell lysis. To the tube one 5 mm steel bead (Qiagen) will be added and then the tube will be placed in a Qiagen TissueLyser LT and disrupted at maximum speed for 2 minutes, in three total cycles. The tubes will then be centrifuged at 14000 g and the supernatant lysate will be collected into another tube and frozen at minus 80° C. until assayed.

Analysis: The tissue lysates and/or supernatant will be analyzed by one of more of the following methods. Multi-plex Analysis: The tissue lysates and supernatants will be assayed using Multiplex (R&D Systems) assay kit containing VEGF-A and IL-1RA. The assay will be processed as per standard kit protocol and read using Luminex® Magpix® multiplex reader. VEGF Reporter Bioassay: VEGF reporter bioassay (Promega) will be performed as per manufacturer instructions and read using a BioTek® Synergy™ HTX plate reader. Briefly, the standard and supernatants will be added to their respective wells and then assay cells will be thawed and added to each well. After six hours of incubation, the luciferase substrate reagent will be added to quantify the functional VEGF present in the medium based on the standard curve. ELISA: The tissue lysates and supernatants will be assayed using HBD-2 ELISA assay kit (Mybiosource, Cat #: MBS9314447). The assay will be processed as per standard kit protocol and read using plate reader.

The results will show that UV light plus hypoxia primed viable placental tissue possesses one or more of the following enhanced therapeutic regenerative properties. An increase in HBD-2 secretion (preferably about a 1 fold to about a 4 fold increase); enhanced angiogenic factor (VEGF-A) function (preferably about a 3 fold to about a 5 fold increase in activity) as determined by a cellular functional assay; an increase in VEGF-A secretion (preferably about a 1 fold to about a 3 fold increase); and/or an increase in IL-1RA secretion (preferably about a 1 fold to about a 3 fold increase) as compared to viable placental tissue not exposed to hypoxic conditions and UV light (non-primed).

### Example 5—In Vivo Testing of Hypoxia Primed Placental Tissue Using the Rat Dermal Ischemic Wound Model

The rat dermal ischemic wound model is designed to mimic wound ischemia by reducing blood flow to wounds through the creation of a bipedicle flap on the rodents' back.

Hypoxia Primed Viable Placental Tissue Mixture (also notated as Flowable Placental Formulation or FPF): Viable human placentas from eligible donors were purchased from The National Disease Research Interchange (NDRI, Philadelphia, PA). The placentas were shipped overnight in cold storage. The placentas were aseptically processed and cleaned using blunt dissection techniques. The umbilical cord (UC), the viable amnion membrane tissue (AM) and viable chorion membrane tissue (CM) were removed from the decidua and cleaned of other maternal tissue including of blood and trophoblast. The UC, CM, and AM were each placed in tissue dishes and then incubated in 250 mL of DMEM low glucose medium plus 0.5% human serum albumin (HSA) in a tissue culture incubator, at 37° C. at 2% 02 (hypoxia) plus 5% CO₂ and 95% RH. After 48 hours, the dishes were removed from the incubator, and the hypoxia primed UC, CM, and AM tissues were collected and blended using a blender (Retsch Knife Mill Grindomix™ GM 200) for 90 seconds forming a mixture of hypoxia primed viable placental tissue (UC, CM, and AM) in minced pieces. The hypoxia primed viable placental tissue mixture (FPF) was diluted in PBS containing 100 mM trehalose and 0.5% HSA in a 1 to 3 ratio by volume, and then 1 ml of the tissue mixture was dispensed in a 10 ml vial and lyophilized using a lyophilization machine (SP Scientific, Lyostar™ 2). The lyophilized vial was resuspended in 1 ml saline solution immediately before in vivo administration.

Animals: A total of 10 rats, 12-14-week-old 400-500 g (Jackson Laboratories, Bar Harbor, ME), were used in this study and housed at the Noble Life Sciences vivarium (Noble Life Sciences, Sykesville, MD). Experimental protocols were approved by the Noble's Institutional Animal Care and Use Committee. All procedures were performed in accordance with the guidelines and regulations of The Association for Assessment and Accreditation of Laboratory Animal Care International. All rats were given Harlan Teklad Rodent Diet (Envigo, Madison, WI) ad libitum.

Dermal Ischemia wound model: For the ischemic bipedicle flap procedure, the hair of animals on the back were removed by first using clippers (from the base of the neck down approximately 11 cm) and then with Nair™ lotion one day before the surgery. Sterile surgical ruler was used to outline the flap with a surgical marking pen (stencil with permanent marker, the outline for the 3.0 cm×10.5 cm flap). Markings were made in the center of the flap at 5.0 cm from the top (centered along the spinal column and placed between the base of the scapulae and the iliac crest) to aid in wound placement. The rat's dorsa was cleaned with alcohol and sprayed liberally with topical Betadine™, and the entire surgery was performed under aseptic technique. Two adjacent 8 mm full-thickness excisional wounds were created on the vertical midline of the flap using a sterile 8 mm disposable biopsy punch. The depth of excision was down to, but did not rupture the anterior fascia of the panniculus carnosus. The full-thickness punch biopsy, including skin and panniculus carnosus muscle, was removed by dissecting with scissors between the panniculus carnosus and the fascia. The dorsal bipedicle skin flap was raised in the craniocaudal direction deep to the panniculus carnosus muscle. Precut and sterilized nonreinforced 0.01 in. thickness Sil-Tec™ medical grade sheeting was placed underneath the flap. The skin flaps and silicone sheet were sutured to the adjacent skin edges with interrupted nonabsorbable sutures to prevent movement of the silicone sheet. Tegaderm™ was applied to cover the entirety of the wound and bipedicle skin flap. Animals were left on heating pad to recover from anesthesia. Post-operative analgesic buprenorphine, 5 mg/kg was administered 6 h post initial injection on the operative day and twice a day for 2 additional days. Baytril™, 5 mg/kg was administered once a day for 5 days to prevent any infection. Rats were subcutaneously injected with 100 μl of either vehicle control (saline) or hypoxia primed viable placental tissue mixture (FPF) around the periphery of the wound on the day of surgery. Confirmation of the creation in ischemic bipedicle flap region was done by visualizing blood flow in the bipedicle flap and ischemic wounds High Resolution Laser Doppler Imager (Moor Instruments, Wilmington, DE) before and after the ischemia procedure. Doppler image post-surgery was acquired 10 mins post-procedure. Photographic wound images were captured on Day 0, Day 4, Day 7, Day 11, Day 14, Day 18, Day 21, Day 25 and Day 28 and wound areas traced using ImageJ (NIH, Bethesda, MD). During photographic and Doppler imaging, the rats were anesthetized and maintained under anesthesia using isoflurane. At 4 weeks post-ischemia, all rats were euthanized, and the healed wound region were harvested for further histopathological analysis.

Histological and immunohistochemical assessment: Collected wound tissue samples were fixed in 10% formalin. Tissue sectioning and staining were performed by Premier Laboratory (Boulder, CO) using standard protocols for hematoxylin and eosin (H&E), Masson's trichrome (MT), Myeloperoxidase (MPO), CD163, CD68, α-smooth muscle actin (αSMA). H&E and MT slides were imaged using Aperio ScanScope™ AT2 (Leica biosystems, Buffalo Grove, IL) and Immunofluorescent (IF) slides were imaged using Vectra™ 3 Automated Quantitative Pathology Imaging System (Akoya Biosciences, Marlborough, MA, USA), and assessed by a blinded independent pathologist.

Samples for immunohistochemistry (IHC) were sectioned at 5 μm and mounted onto charged slides. Slides were dried overnight, baked at 60° C. for 1 h, deparaffinized in xylene, rinsed in alcohol, rehydrated in water, and equilibrated in wash buffer (TRIS buffered saline with 0.05% Tween-20; Dako, K8007). Heat-induced epitope retrieval (HIER) was performed in a Dako PT Link using a TRIS/EDTA buffer (FLEX TRS High, pH 9; Dako, #K8004). A 20-min 95° C. retrieval was utilized prior to αSMA, CD31, and MPO staining. Slides were then cooled and rinsed in wash buffer and the remaining IHC steps were carried out at room temperature on an Autostainer PlusLink stainer (Dako). Proteolytic induced epitope retrieval (PIER) using Proteinase K for 5 minutes (Dako, S3020) was performed prior to CD68, CD163, and Collagen IV staining. Within the autostainer, serum-free protein block was applied for 5 min (Dako, #X0909).

The following primary antibodies and concentrations were used: for a smooth muscle actin (αSMA), rabbit polyclonal anti-αSMA primary antibody for 45 min (0.33 pg/mL) (Abcam, #ab5694); for CD31, mouse monoclonal [TLD-3A12] anti-CD31 primary antibody for 45 min (10 pg/mL) (Invitrogen #MA1-80069); for CD68, mouse monoclonal [ED-1] anti-CD68 primary antibody for 30 min (5 pg/mL) (Biorad #MCA341); for CD163, mouse monoclonal [ED-2] anti-CD163 primary antibody for 30 min (6.67 pg/mL) (Biorad #MCA342); for Collagen IV, rabbit polyclonal anti-Collagen IV primary antibody for 60 min (10 μg/mL) (Invitrogen #PA1-28534); for MPO, rabbit polyclonal anti-MPO primary antibody for 30 min (1.33 μg/mL) (Abcam #ab9535). The following secondary antibodies and concentrations were used; for αSMA, goat anti-rabbit IgG Alexa Fluor 488 for 30 min (5 μg/mL) (Invitrogen #A11008); for CD31, CD68, and CD163, goat anti-mouse IgG1 Alexa Fluor 568 for 30 min (5 μg/mL) (Invitrogen #A21124); for Collagen IV and MPO, goat anti-rabbit IgG Alexa Fluor Plus 555 for 40 minutes (6.67 μg/mL) (Invitrogen #A32732). Wash buffer rinses were carried out between appropriate reagents. All slides were then manually counterstained for 5 min with DAPI (Invitrogen, #D1306). The slides were rinsed in deionized water and coverslipped with Prolong Diamond Antifade Mountant (Invitrogen #P36970).

Cytokine/Chemokine multiplex analysis of necropsied animal tissue samples: Collected frozen samples of wound tissues were sent to RayBiotech (Peachtree Corners, GA) for cytokine/chemokine analysis. Tissues were lysed and extracts prepared according to RayBiotech's standard tissue homogenization procedures. Tissues were lysed in the presence of T-PER (Thermo Fisher Scientific, Waltham, MA) supplemented with a protease inhibitor cocktail (Sigma-Aldrich, St. Louis, MO). Clarified supernatants, obtained post-centrifugation at 14000 rpm for 5 min, were analyzed for the presence of cytokines and chemokines using the Quantibody Rat Cytokine Array (Cat #QAR-CAA-67). The concentration of each analyte was normalized to total protein concentration of the sample.

MicroRNA array and PCR gene expression analysis: Flash-frozen skin tissues collected post-wound closure were sent to Qiagen Inc (Germantown, MD) for Quantitative RT-PCR analysis (RT 2 PCR) and microRNAs (miRs) qPCR analysis. RNA was isolated from flash frozen tissues using the miRNeasy mini kit (QIAGEN) according to the manufacturer's standard procedure (QIAGEN, MD). RT 2 PCR rat wound healing array was performed by QIAGEN according to their standard protocol for Cat #PARN-121Z. For miRs analysis, QIAGEN used the extremely specific and sensitive LNA technology and the miRCURY LNA miRNA PCR System to run panel I+II for 752 miRs (Cat #YAMR-312Y).

Statistical analysis: Graphpad™ Instat Software (Graphpad, La Jolla, CA, USA) was used for statistical analysis and plotting graphs. Results are presented as mean±SD. Mann-Whitney test was used to determine the significance of differences between groups, whereby p<0.05 was considered significant. A cutoff of P-value<0.05 to pass a Benjamini-Hochberg correction was performed to increase stringency for the gene array analysis. Student's T-test was used to determine the significance of differences between groups, whereby p<0.05 was considered significant.

### Results

Hypoxia Primed Viable Placental Tissue Mixture (FPF) Accelerates Ischemic Wound Healing and Closure with Improved Tissue Architecture

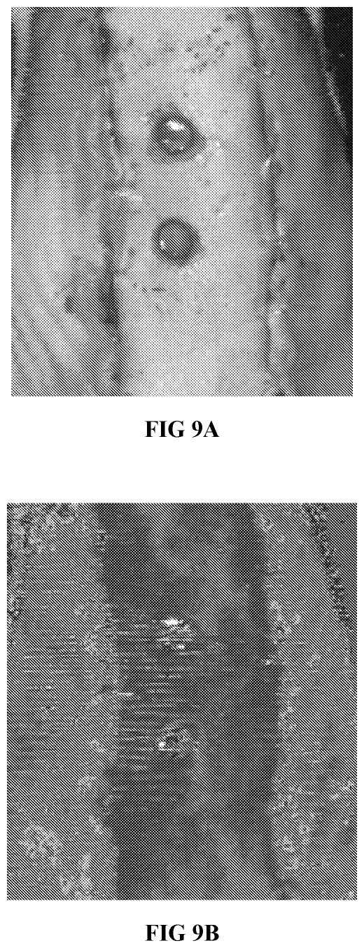

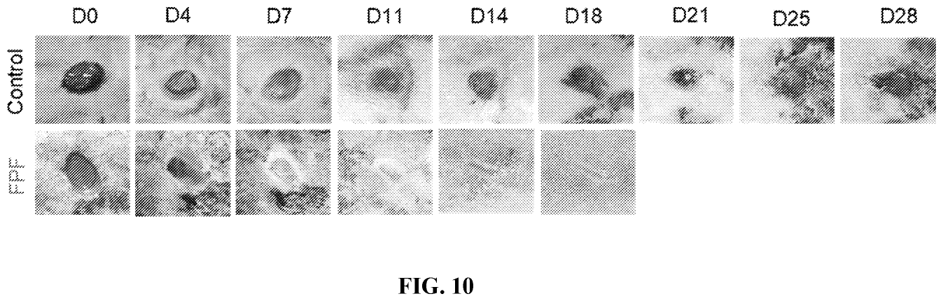

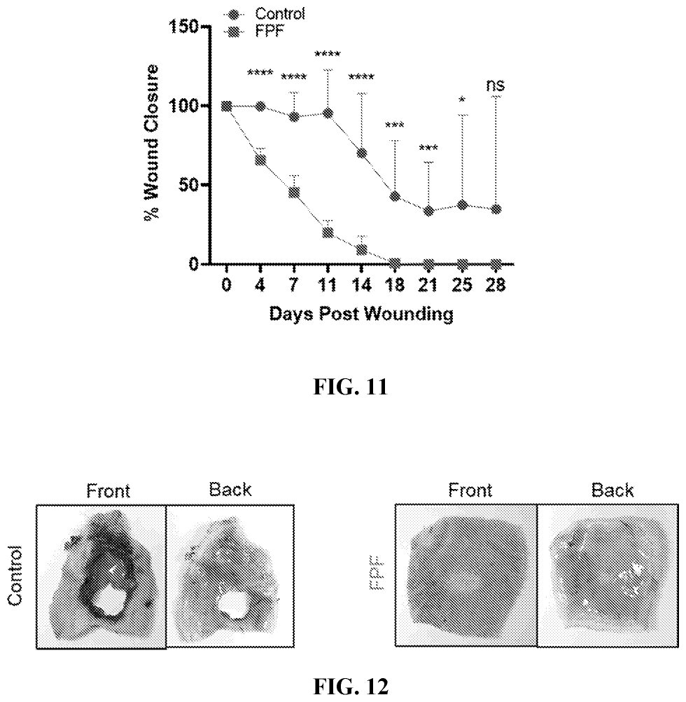

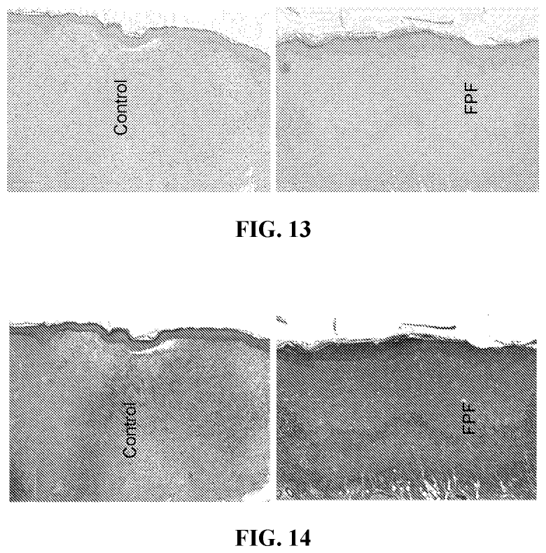

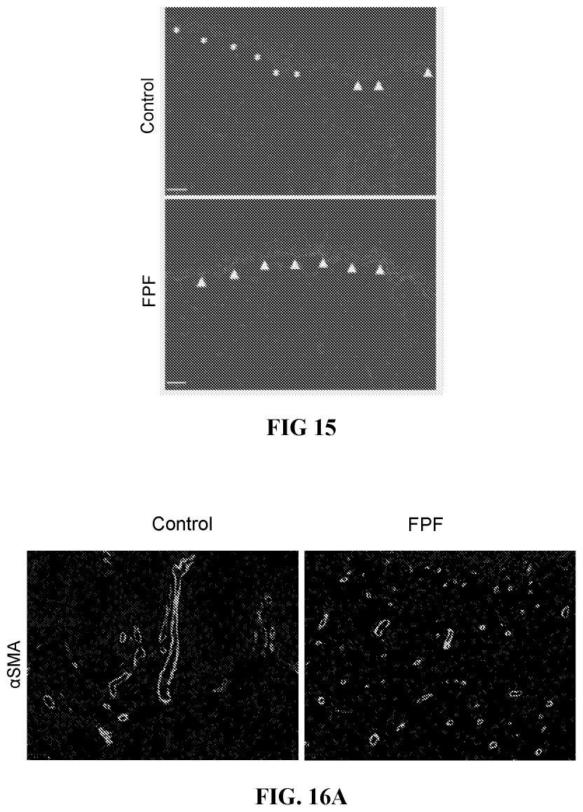

Confirmation of tissue ischemia was achieved by use of Doppler Imager. While the photo image (FIG. 9A) shows the creation of two wounds on the skin flap, the doppler image (FIG. 9B) demonstrates a clear contrast in perfusion between the flap and the adjacent skin. The entire flap displayed lack of blood supply to the flap from the incised edges, while the wounds were created in the ischemic section. Treatment with FPF resulted in total closure of ischemic wounds as early as 18 days post-wounding whereas control wounds remained open at 28 days post-wounding (FIG. 10). Significant acceleration in rate of ischemic wound healing was observed by the decrease in wounds area of FPF treated wounds as compared to the open control wounds (FIG. 11 and FIG. 12). Further, histological evaluation of FPF treated wounds revealed proper granulation and re-epithelialization consistent with a normal rat dermis and epidermis in contrast to the control wounds that achieved closure (FIG. 13 and FIG. 14). H&E staining of tissue shows that the FPF treated animals showed appropriate level of tissue healing and the absence of epidermal hyperplasia and improper granulation tissue formation as were observed in the control animals healed tissue sections (FIG. 13). Masson's Trichrome staining further corroborated healthy deposition and maturation of collagen in FPF treated wounds (FIG. 14). Furthermore, immunostaining for Collagen IV, a component of basal lamina, was continuous and well-formed in FPF treated wound but was tortuous and blunted in controls (FIG. 15).

Hypoxia primed viable placental tissue mixture (FPF) treatment is conducive for a pro-angiogenic and an anti-inflammatory wound microenvironment

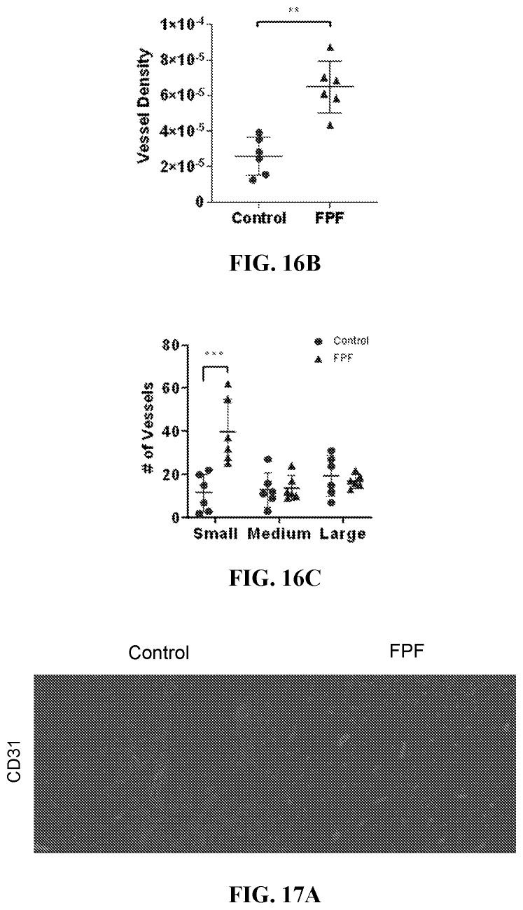

To examine whether FPF supports tissue vascularization, we evaluated the healed tissue sections for blood vessel cell markers: smooth muscle actin, αSMA (FIG. 16A), and endothelial cell marker, CD31 (FIG. 17A). Both αSMA and CD31 were markedly increased in FPF-treated wounds (FIG. 16A and FIG. 17A). αSMA- (FIG. 16B) and CD31-positive (FIG. 17B) blood vessels were representative of the formation of more blood vessels in FPF-treated than in control wound. CD31 stain colocalized in αSMA stained blood vessels (FIG. 18). Furthermore, we evaluated the vessel results to visualize vessel size distribution by grouping vessels into either small (<500 μm), medium (500-1,000 μm) or large (>1,000 μm) diameter. This data showed that FPF-treated wounds had similar density of large or medium sized blood vessels, but significantly greater density of small blood vessels compared with control wounds (FIG. 16C and FIG. 17C).

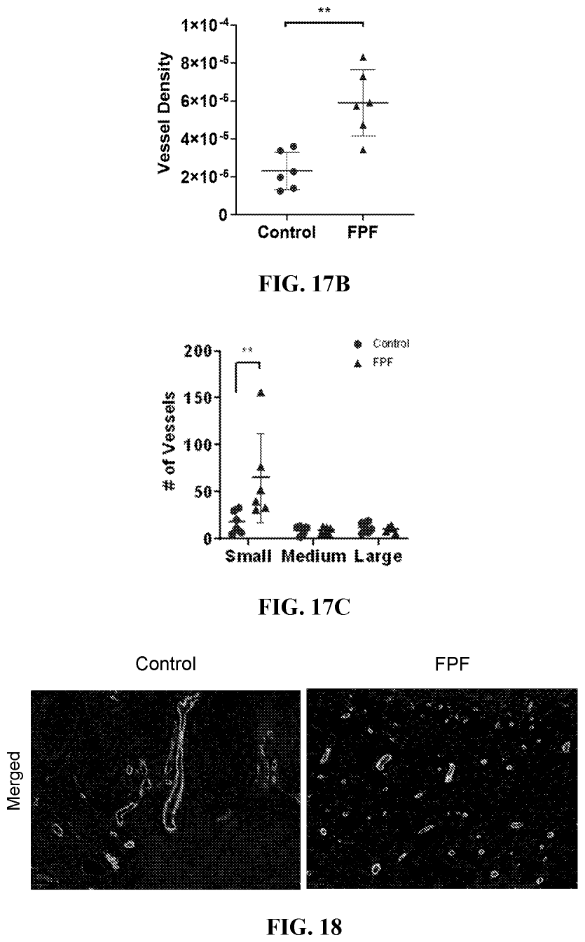

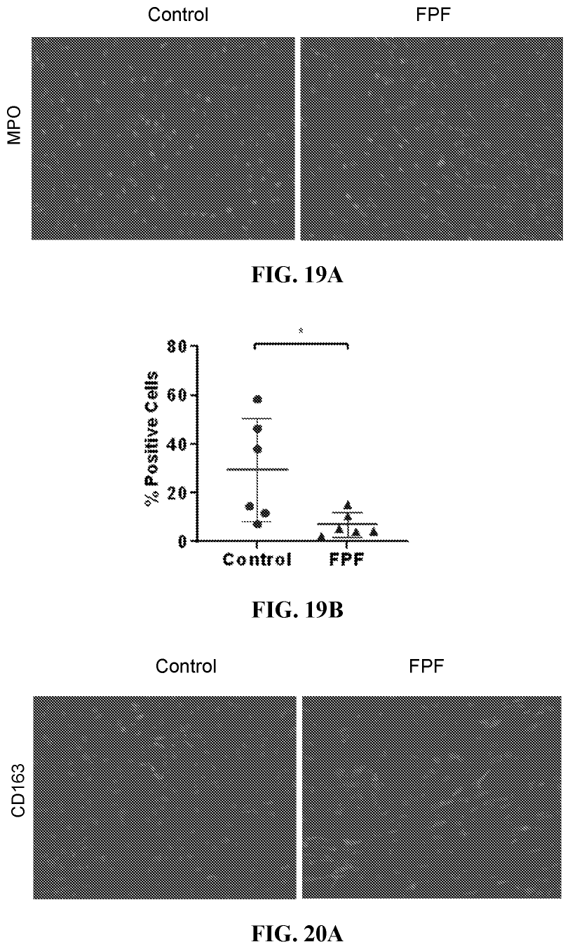

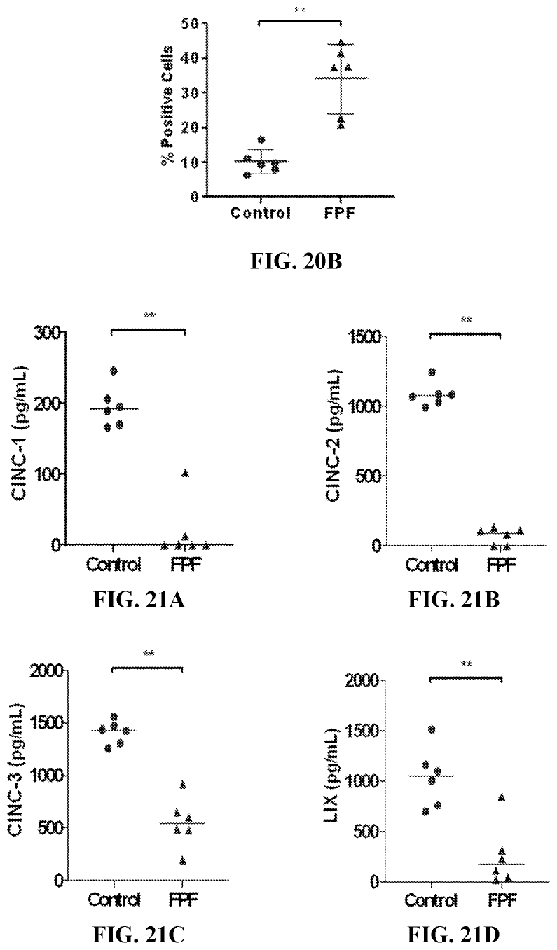

Tissue sections were stained for neutrophil marker myeloperoxidase, MPO (FIG. 19A) and M2-macrophage marker, CD163 (FIG. 20A). DAPI (blue staining) was used to visualize cell nuclei. We show that the FPF treated animals present significantly lower levels of neutrophils as compared to the control healed wounds (FIG. 19A and FIG. 19B). Influx of neutrophils in wound healing is followed by monocyte/macrophages. We observed a significantly higher number of pro-resolving/anti-inflammatory M2-macrophages in the FPF treated wounds (FIG. 20A and FIG. 20B). Overall, our findings of the FPF treated healed wounds suggest that accelerated and appropriate ischemic wound healing could be due to the observed changes in wound microenvironment.

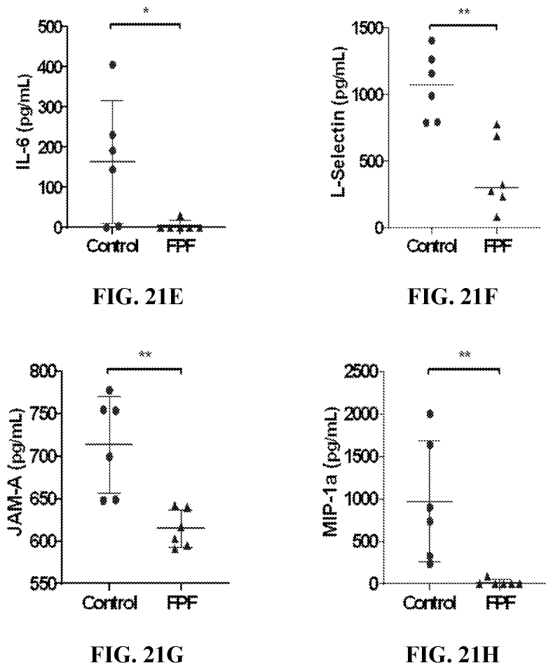

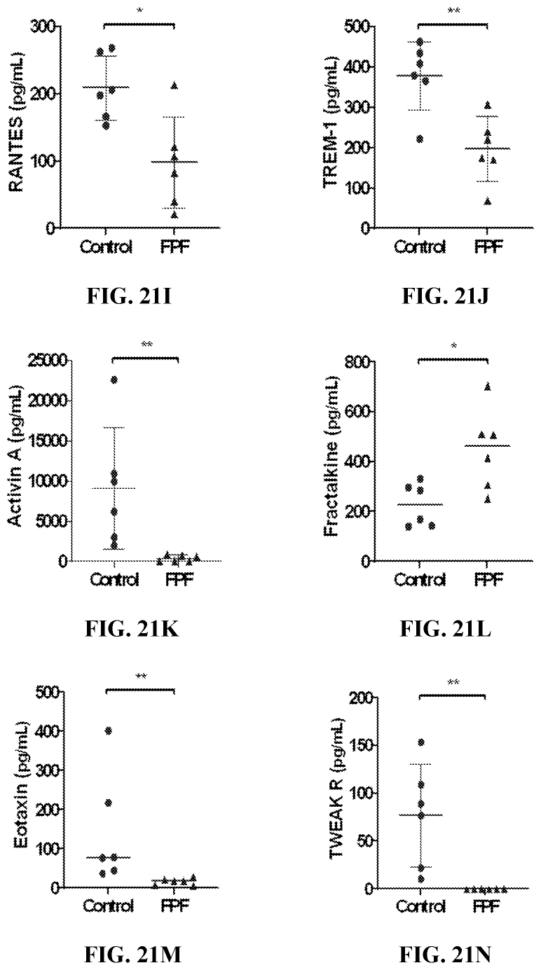

To elucidate the observed differences in inflammatory cells, we evaluated the cytokine/chemokine levels in the wound tissue by a multiplex signaling protein array (FIGS. 21A through 21N). We show that FPF treatment significantly reduced the levels of important pro-inflammatory chemoattractants: cytokine-induced neutrophil chemoattractant-1 (CINC-1) (FIG. 21A), cytokine-induced neutrophil chemoattractant-2 (CINC-2) (FIG. 21B), cytokine-induced neutrophil chemoattractant-1 (CINC-3) (FIG. 21C), lipopolysaccharide-inducible CXC chemokine (LIX) (FIG. 21D), interleukin 6 (IL-6) (FIG. 21E), L-selectin (FIG. 21F), junctional adhesion molecule A (JAM-A) (FIG. 21G), macrophage inflammatory protein-α (MIP-1a) (FIG. 21H), regulated upon activation, normal T cell expressed and presumably secreted (RANTES) (FIG. 21I), triggering receptors expressed on myeloid cells-1 (TREM-1) (FIG. 21J), activin A (FIG. 21K), eotaxin (FIG. 21M), tumor necrosis factor-(TNF-) including weak inducer of apoptosis (TWEAK R) (FIG. 21N), and fractalkine (FIG. 21L). Taken together, the observed consequences in FPF treated wounds are suggestive of an anti-inflammatory granulation tissue microenvironment.

### Hypoxia Primed Viable Placental Tissue Mixture (FPF) Induces Transcriptional Regulation Favorable for Ischemic Wound Healing

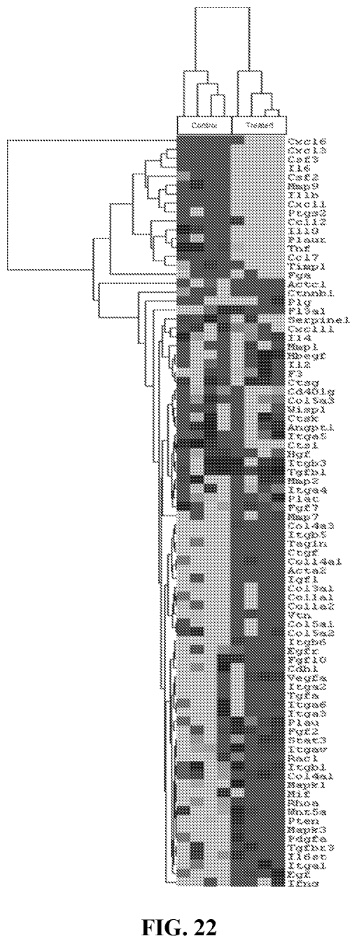

Gene array analysis of the collected healed tissue from both control and FPF-treated animals was performed to elucidate the wound healing gene signatures. The gene array included 84 rat wound healing associated genes. The non-supervised hierarchical clustering of genes with a heat map illustrates the fold change in genes expressed in control vs FPF-treated wounds (FIG. 22). The criterion used to screen down- or up-regulated genes was fold change of ≥2 and a Benjamini-Hochberg adjusted p value≤0.05 to increase strictness during panel profiling. Individual genes with the most significant over- or under-expressed after FPF treatment are listed in Table 1.

TABLE 1

| | | |
|---|---|---|
| Gene Symbol | Fold Regulation | BH p-value |

Genes Over-Expressed

| | | |
|---|---|---|
| Col4a3 | 2.309693 | 0.019032 |
| Itgb6 | 2.230203 | 0.022504 |

Genes Under-Expressed

| | | |
|---|---|---|
| Ccl12 | −24.194614 | 0.024615 |
| Ccl7 | −13.027828 | 0.019032 |
| Csf2 | −49.694860 | 0.045848 |
| Csf3 | −194.82157 | 0.019032 |
| Cxcl1 | −48.229749 | 0.019032 |
| Cxcl3 | −277.51132 | 0.019032 |
| Il1b | −55.857964 | 0.019032 |
| IL6 | −181.135527 | 0.019032 |
| Mmp9 | −76.35755 | 0.028479 |
| Plaur | −9.4331041 | 0.030431 |
| Ptgs2 | −40.155383 | 0.034759 |
| Serpine1 | −6.4475728 | 0.034759 |
| Tnf | −6.9522493 | 0.045848 |

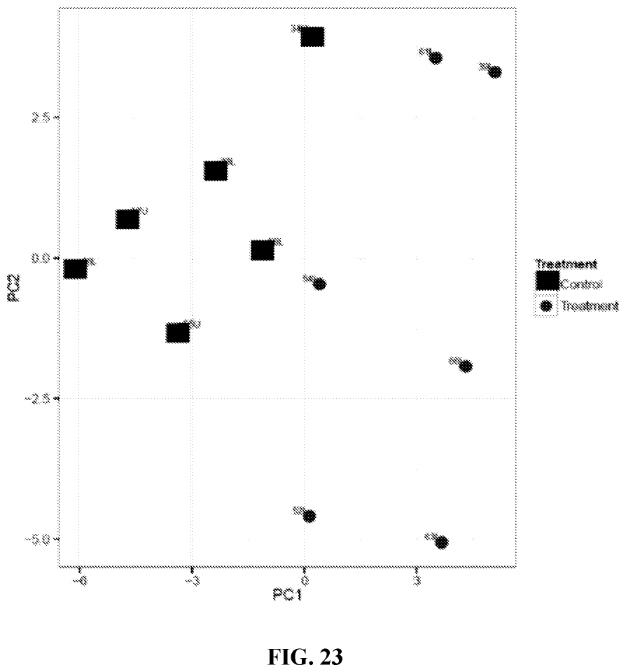

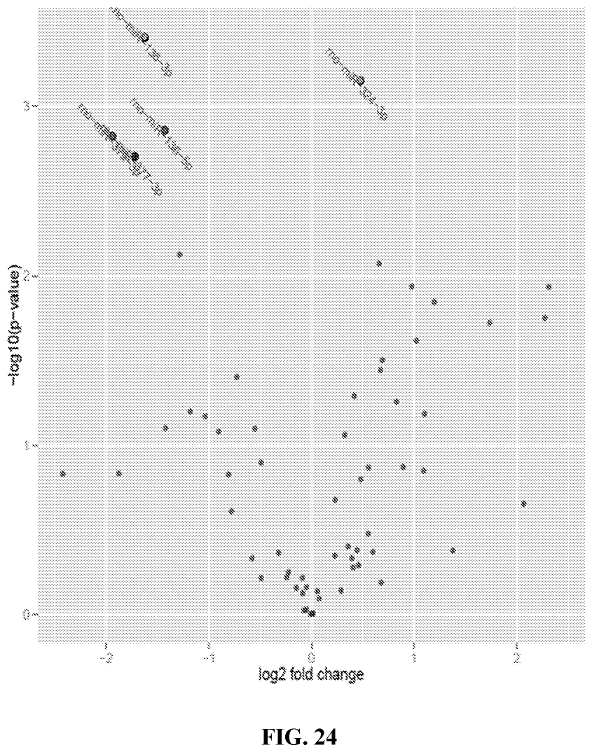

Increasing evidence suggest that microRNA (miR) dysregulation leads to altered gene expression and outcomes in chronic wound pathogenesis and healing progression. To illuminate this possibility, we performed extremely specific and sensitive miRNA profiling using LNA-enhanced, SYBR® Green-based miRNA qPCR to identify and compare tissue-specific miR expression in FPF-treated and control ischemic wounds. A principal component analysis (PCA) was performed to identify two principal components (PC1 and PC2) (FIG. 23). The readily apparent independent clusters for control and FPF-treated groups was observed with the separation within the two groups (FIG. 23). Statistically significant differentially-expressed miRs were identified from the constructed Volcano Plot (FIG. 24). The miRs with high statistical significance can be identified in two regions, the top of the plot and the left and right of the center of the plot. The criterion used to screen down- or up-regulated miRs was fold change of ≥2 and a p value<0.05. Based on these parameters, the most differentially downregulated known miRs, are mir-136-3p, miR-136-5p, miR-379-3p, miR-377-3p, miR-409a-3p as shown in Table 2. Upregulated differential expression was observed in miR-25-5p, miR-223-3p, miR-146b-5p, miR-346, miR-501-5p, and miR-466c-5p as shown in Table 2.

TABLE 2

| miR Name | Fold Change | P-value |
|---|---|---|
| rno-mir-136-3p | −3.1 | 0.00039 |
| rno-miR-136-5p | −2.7 | 0.0014 |
| rno-miR-379-3p | −3.8 | 0.0015 |
| rno-miR-377-3p | −3.3 | 0.0020 |
| rno-miR-409a-3p | −2.4 | 0.0075 |
| rno-miR-25-5p | 2.0 | 0.012 |
| rno-miR-223-3p | 5.0 | 0.012 |
| rno-miR-146b-5p | 2.3 | 0.014 |
| rno-miR-346 | 4.8 | 0.018 |
| rno-miR-501-5p | 3.3 | 0.019 |
| rno-miR-466c-5p | 2.0 | 0.024 |

These differentially-expressed miRs have been identified for their role in angiogenesis, regulation of inflammatory microenvironment, cell migration and apoptosis, reactive oxygen species generation, and restoring epithelial barrier function, all that are altered in chronic wound. Hence, the data suggests that FPF favorably modulates miRs that help in redirecting tissue healing conducive for appropriate restoration of the dermal tissue.

These results show that the hypoxia primed viable placental tissue mixture (FPF) is conducive to pushing ischemic chronic wounds onto a path of regenerative tissue healing processes, to accelerate ischemic wound healing, and to achieve fitting wound closure.

### Example 6—In Vivo Testing Using the Rat Hind Limb Ischemia Model

Peripheral arterial diseases (PAD) is caused by the chronic reduction in perfusion and can be mimicked using a rat hindlimb ischemia model.

Hypoxia Primed Viable Placental Tissue Mixture (also notated as Flowable Placental Formulation or FPF): Viable human placentas from eligible donors were purchased from The National Disease Research Interchange (NDRI, Philadelphia, PA). The placentas were shipped overnight in cold storage. The placentas were aseptically processed and cleaned using blunt dissection techniques. The umbilical cord (UC), the viable amnion membrane tissue (AM) and viable chorion membrane tissue (CM) were removed from the decidua and cleaned of other maternal tissue including of blood and trophoblast. The UC, CM, and AM were each placed in tissue dishes and then incubated in 250 mL of DMEM low glucose medium plus 0.5% human serum albumin (HSA) in a tissue culture incubator, at 37° C. at 2% 02 (hypoxia) plus 5% CO₂ and 95% RH. After 48 hours, the dishes were removed from the incubator, and the hypoxia primed UC, CM, and AM tissues were collected and blended using a blender (Retsch Knife Mill Grindomix™ GM 200) for 90 seconds forming a mixture of hypoxia primed viable placental tissue (UC, CM, and AM) in minced pieces. The hypoxia primed viable placental tissue mixture (FPF) was diluted in PBS containing 100 mM trehalose and 0.5% HSA in a 1 to 3 ratio by volume, and then 1 ml of the tissue mixture was dispensed in a 10 ml vial and lyophilized using a lyophilization machine (SP Scientific, Lyostar™ 2). The lyophilized vial was resuspended in 1 ml saline solution immediately before in vivo administration.

Animals: A total of 10 rats, 12-14-week-old 400-500 g (Jackson Laboratories, Bar Harbor, ME) were used in this study and housed at the Noble Life Sciences vivarium (Noble Life Sciences, Sykesville, MD). Experimental protocols were approved by the Noble's Institutional Animal Care and Use Committee. All procedures were performed in accordance with the guidelines and regulations of The Association for Assessment and Accreditation of Laboratory Animal Care International. All rats were fed Harlan Teklad Rodent Diet (Envigo, Madison, WI) ad libitum.

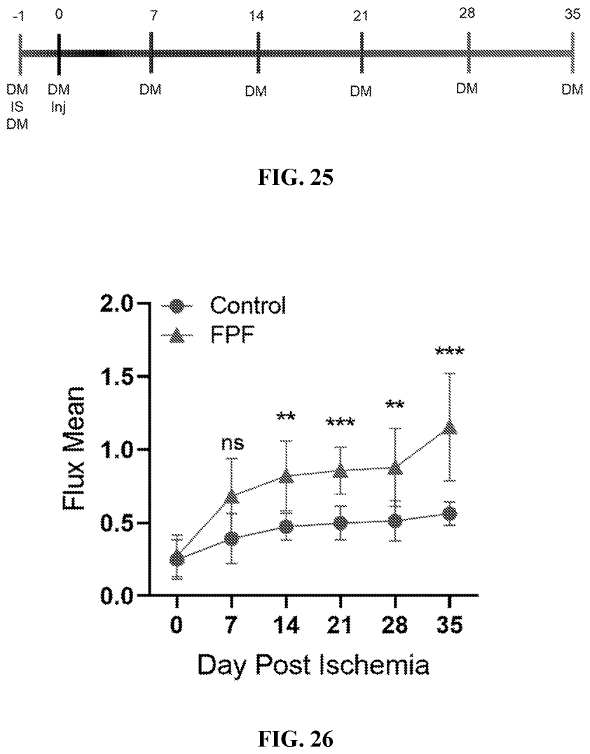

Hind Limb Ischemia model: For hind limb ischemia, the animals were anesthetized and maintained under anesthesia using isoflurane inhalation. The depth of anesthesia was assessed by lack of withdraw reflex with toe pinch. Buprenorphine (analgesic, 5 mg/kg) and Baytril™ (5 mg/kg, antibiotic) was subcutaneously administered before the start of the surgery. Surgical site on the hind limb was shaved and depilated to remove hair. Animals were placed on a heating pad to avoid hypothermia. Ophthalmic ointment/gel in both eyes of the animal was applied to prevent corneal desiccation. Sterile gauzes soaked in 70% ethanol were used to disinfect and clean the shaved region. Subcutaneous injection of 1% Lidocaine was done along the incision site. For hind limb ischemia, after the skin incision, the entire femoral artery (superficial, deep, and common femoral vessel) and all its major branches were ligated and excised. The external iliac artery and all of the above arteries were ligated with 3-5-0 silk suture and cauterized. Finally, the femoral artery was excised from its proximal origin as a branch of the external iliac artery, to the point distally where it bifurcates into the saphenous and popliteal arteries. As a consequence, blood flow to the ischemic limb becomes completely dependent upon collateral vessels issuing from the internal iliac artery. The incision site and skin layer were closed by a continuous suture using Ethibond™ suture 5-0. Post-operative analgesic buprenorphine, 5 mg/kg was administered 6 h post initial injection on the operative day and twice a day for 2 additional days. Baytril™, 5 mg/kg was administered once a day for 5 days to prevent any infection. Confirmation of the creation in ischemic bipedicle flap region was done by visualizing blood flow in the bipedicle flap and ischemic wounds using High Resolution Laser Doppler Imager (Moor Instruments, Wilmington, DE) before and after the ischemia procedure. Images post-surgery were acquired 10 minutes post-procedure. Rats were injected one day post-surgery with vehicle control (saline) or with hypoxia primed viable placental tissue mixture (FPF) via intramuscular injection on the same hind limb that underwent ischemic surgery. Blood flow in both the hind limbs were measured using High Resolution Laser Doppler Imager (Moor Instruments, Wilmington, DE) before ischemia procedure, and then on Day 1 post-surgery. Following Doppler images were acquired on Day 3, Day 7, Day 14, Day 21, Day 28, and Day 35 post-treatment. The study timeline is shown in FIG. 25 and shows the ischemia surgery time point (IS), the injection time point (Inj), and doppler measurement timepoints (DM). During Doppler imaging, the rats were anesthetized and maintained under anesthesia using isoflurane. At 5 weeks post-ischemia, all rats were euthanized and the gracilis muscle harvested for further histopathological and molecular analysis.

Cytokine/Chemokine multiplex analysis of necropsied animal tissue samples: Collected frozen samples of hindlimb Gracilis muscle were sent to RayBiotech (Peachtree Corners, GA) for cytokine/chemokine analysis. Tissues were lysed and extracts prepared according to RayBiotech's standard tissue homogenization procedures. Tissues were lysed in the presence of T-PER (Thermo Fisher Scientific, Waltham, MA) supplemented with a protease inhibitor cocktail (Sigma-Aldrich, St. Louis, MO). Clarified supernatants, obtained post-centrifugation at 14000 rpm for 5 min, were analyzed for the presence of cytokines and chemokines using the Quantibody Rat Cytokine Array (Cat #QAR-CAA-67). The concentration of each analyte was normalized to total protein concentration of the sample.

Statistical analysis: Graphpad™ Instat Software (Graphpad, La Jolla, CA, USA) was used for statistical analysis and plotting graphs. Results are presented as mean±SD. Student's T-test was used to determine the significance of differences between groups, whereby p<0.05 was considered significant.

### Results: Hypoxia Primed Viable Placental Tissue Mixture (FPF) Increases Hind Limb Tissue Perfusion Upon Ischemic Tissue Injury

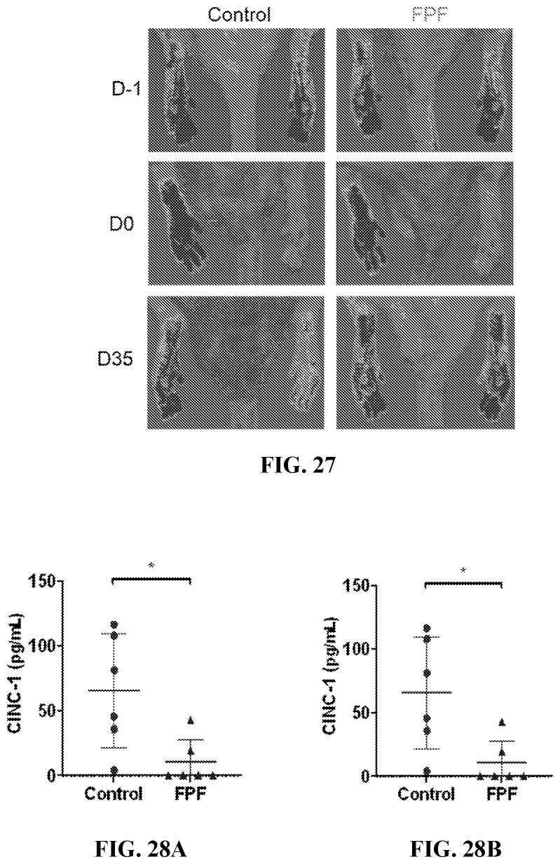

Peripheral arterial diseases (PAD) are caused by the chronic reduction in perfusion and was mimicked using a rat hindlimb ischemia model. Vehicle control or FPF was injected a day after ischemia was induced and tissue perfusion was measured using High Resolution Laser Doppler Imager as shown in the study timeline in FIG. 25. This methodology resulted in longitudinal assessment of full-field analysis of every rat. Percent flux of the ischemic limb to the healthy limb was calculated after the rats were stabilized under anesthesia for 10 minutes. As can be seen in FIG. 26 and FIG. 27, the treatment with FPF resulted in significant increases in percent perfusion compared to saline group as early as 14 days and the improvement maintained significantly higher than control animals through the course of the study. These results show that angiogenic and pro-healing factors present in hypoxia primed viable placental tissue mixture (FPF) stimulated an increase in hindlimb tissue perfusion that helped restore perfusion to normal levels.

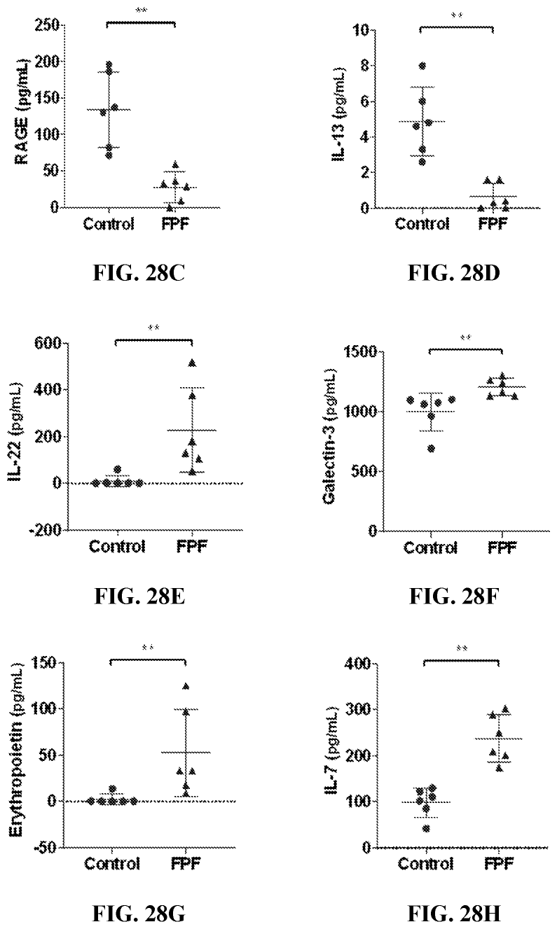

To evaluate the effects of FPF in the hindlimb, we next sought to analyze the key cytokine biomarkers using a multiplex protein array in the gracilis muscle post-study completion at Day 35 (FIGS. 28A through 28H). The analysis included cytokine-induced neutrophil chemoattractant-1 (CINC-1) (FIG. 28A), cytokine-induced neutrophil chemoattractant-2 (CINC-2) (FIG. 28B), receptor for advanced glycation end products (RAGE) (FIG. 28C), interleukin-13 (IL-13) (FIG. 28D), interleukin-22 (IL-22) (FIG. 28E), galectin-3 (FIG. 28F), erythropoietin (FIG. 28G) and interleukin-7 (IL-7) (FIG. 28H). FPF treated animals showed a significant decrease in pro-inflammatory factors (FIGS. 28A through 28D), and an increase in anti-inflammatory, pro-angiogenic, and ischemia protective factors (FIGS. 28E through 28H).

1. A composition comprising hypoxia primed viable placental tissue.

2. The composition of claim 1, wherein the hypoxia primed viable placental tissue exhibits enhanced angiogenic factor (VEGF-A) function as determined by a cellular functional assay, an increase in VEGF-A secretion, and/or an increase in IL-1RA secretion as compared to non-primed viable placental tissue cultured under normoxia conditions.

3. The composition of claim 1, wherein the hypoxia primed viable placental tissue is amnion tissue, chorion tissue, or umbilical cord tissue, or mixtures thereof.

4. The composition of claim 1, wherein the hypoxia primed viable placental tissue comprises one or more of MSCs, epithelial cells, or fibroblasts.

5. The composition of claim 1, wherein the hypoxia primed viable placental tissue is in the form of a sheet, wrap, or graft.

6. The composition claim 1, wherein the hypoxia primed viable placental tissue is in the form of minced pieces or a powder.

7. The composition claim 6 wherein the composition further comprises a pharmaceutically acceptable carrier.

8. The composition of claim 7, wherein the pharmaceutically acceptable carrier is a suspension, solution, gel, paste, emulsion, cream, or powder.

9. The composition of claim 7, wherein the pharmaceutically acceptable carrier comprises one or more of a saline solution, a buffer solution, a sugar, trehalose, a protein, or a starch.

10. The composition of claim 1, wherein the composition further comprises one or more bioactive materials.

11. The composition of claim 10, wherein the one or more bioactive materials comprise extracellular vesicles, exosomes, microvesicles, secretomes, cytokines, growth factors, peptides, antimicrobial peptides, extracellular matrices, TNF-alpha, interferon-gamma, or nanoparticles thereof.

12. The composition of claim 10, wherein the one or more bioactive materials is a byproduct of a hypoxia priming process.

13. The composition of claim 1, wherein the composition is cryopreserved or lyophilized.

14. A method of regenerating, replacing, or repairing diseased, damaged, or injured body tissue of a subject, the method comprising administering to the subject the composition of claim 1.

15. The method of claim 14, wherein the diseased, damaged or injured body tissue comprises tendons, cartilage, ligaments, periosteum, perichondrium, synovium, fascia, mesentery, sinew, dental tissue, gums, fistulas, nasal septum, vaginal wall tissue, abdominal wall tissue, peritoneum, tumor resection sites, dermal tissue, dermal wounds, dermal lesions, dermal abrasions, dermal burns, first degree burns, partial thickness burns, full thickness burns, epidermal wounds, congenital wounds, toxic epidermal necrolysis, epidermolysis bullosa, pyoderma gangrenosum, dermal ulcers, diabetic ulcers, diabetic foot ulcers, venous ulcers, venous let ulcers, pressure ulcers, arterial ulcers, decubitus ulcers, stasis ulcers, ischemic ulcers, chronic wounds, acute wounds, surgical wounds, internal wounds, hernia, bladder tissue, keloids, adhesions, organ lacerations, organ defects, diseased organ tissue, epithelial defects, spinal cord tissue, nerve tissue, tympanic membranes, and/or mucous membranes.

16. The method of claim 14, wherein the composition is administered topically, subcutaneously, surgically, or by injection.

17. The method of claim 14, wherein the composition is administered by coating the composition onto the surface of a medical device implant and implanting the coated device into the subject.

18. The composition of claim 1, wherein the hypoxia primed viable placental tissue exhibits:
enhanced angiogenic factor (VEGF-A) function at about a 3 fold to about a 5 fold increase of activity as determined by a cellular functional assay;
an increase in VEGF-A secretion at about a 1 fold to about a 3 fold increase; and/or
an increase in IL-1RA secretion at about a 1 fold to about a 3 fold increase as compared to non-primed viable placental tissue cultured under normoxia conditions.

19. The composition of claim 10, wherein the one or more bioactive materials comprise TNF-alpha at an amount from about 5 to about 50 ng/ml.

20. The composition of claim 10, wherein the one or more bioactive materials comprise interferon-gamma at an amount from about 5 to about 50 ng/ml.
